(19) United States
(12) Patent Application Publication
(10) Pub. No.: US 2025/0297225 A1
(43) Pub. Date: Sep. 25, 2025

(54) MULTIPLE HEART TISSUE CULTURE FUSION

(71) Applicant: IMBA - INSTITUT FÜR MOLEKULARE BIOTECHNOLOGIE GMBH, Wien (AT)

(72) Inventors: Sasha MENDJAN, Wien (AT); Clara SCHMIDT, Wien (AT); Alison DEYETT, Wien (AT); Tobias ILMER, Wien (AT)

(21) Appl. No.: 18/863,475

(22) PCT Filed: Jun. 15, 2023
(86) PCT No.: PCT/EP2023/066125

§ 371 Date: Nov. 6, 2024

(30) Foreign Application Priority Data
Jun. 15, 2022 (EP) 22179283.1

(51) Int. Cl. C12N 5/077; A61K 35/34; C12N 5/071; G01N 33/50

(52) U.S. Cl. CPC C12N 5/0657; A61K 35/34; C12N 5/0697; G01N 33/5061; C12N 2500/38; C12N 2501/115; C12N 2501/155; C12N 2501/16; C12N 2501/33; C12N 2501/415; C12N 2506/04; C12N 2513/00

(57) ABSTRACT

A heart tissue model including a heart tissue with at least one inner cavity or a central chamber, wherein the heart tissue model including at least two different heart tissues selected from left ventricle tissue, right ventricle tissue, atrial tissue, outflow tract tissue, atrioventricular canal tissue, sinoatrial node tissue, and atrioventricular node tissue, wherein the central chamber can be shared by at least two different heart tissues, and wherein the at least two different heart tissues include a calcium signaling connection and/or ability to propagate a tissue contraction-; methods of generating such a tissue model and uses of the tissue model for screening purposes is disclosed.

The present invention relates to the field of heart tissue model generation.

### BACKGROUND

Congenital heart disease (CHD) is the most common human developmental birth defect and the most prevalent cause of embryonic and fetal mortality. CHDs occur most often in specific compartments of the embryonic heart, such as the outflow tract (OFT), atria, atrioventricular canal (AVC), right ventricle (RV) and most rarely in the left ventricle (LV). For about 75% of CHDs, we still do not know the underlying cause. They could originate from undiscovered genetic mutations, environmental factors, or combined effects. To test and study possible causes, we need models representing all the compartments of the developing human heart.

CHDs occur early in embryonic development, often before pregnancy has been detected, making the characterization of defect etiology challenging. These difficulties are compounded by the lacking control over the genetic background and environmental interactions during human embryonic development. Understanding the etiology of human compartment-specific defects is challenging in animals due to the complexity, speed, inaccessibility and species-specific physiological differences. Therefore, recent human self-organizing cardiac organoid models are complementary, offering greater accessibility, reductionist dissection of mechanisms, and high throughput statistical significance capability. However, these systems do not allow the mechanistic dissection of defects representing all the interacting compartments (OFT, AVC, atria, RV and LV) of the human embryonic heart.

WO 2019/174879 A1 describes an artificial heart tissue organoid that is grown into a multi-layered aggregate that contains large amounts of non-cardiac cells, such as foregut endoderm cells.

Hofbauer et al. (Cell 2021; 184 (12): 3299-3317. e22) and WO 2021/186044 A1 disclose the generation of a left-ventricular cardiac organoid (cardioid) by self-organization. Although the self-organization after initiation of cardiac differentiation using specific drifting factors improves on prior artificial cardiac tissues, this publication still does not show the diversity of in vivo heart development.

It is therefore a goal to provide different and more advanced artificial heat tissues that are capable to model different or a greater diversity of the developing heart.

### SUMMARY OF THE INVENTION

The invention provides a heart tissue model comprising a heart tissue with at least one inner cavity or a central chamber, wherein the heart tissue model comprises at least two different heart tissues selected from left ventricle tissue, right ventricle tissue, atrial tissue, outflow tract tissue, atrioventricular canal tissue, sinoatrial node tissue, and atrioventricular node tissue, wherein the central chamber can be shared by at least two different heart tissues, and/or wherein the at least two different heart tissues comprise a calcium signaling connection and/or ability to propagate a tissue contraction. Also provided is a heart tissue model comprising a heart tissue with a central chamber, wherein the central chamber can be shared by at least two different heart tissues selected from left ventricle tissue, right ventricle tissue, atrial tissue, outflow tract tissue, atrioventricular canal tissue, sinoatrial node tissue, and atrioventricular node tissue.

The invention further provides a method to generate a heart tissue model, the method comprising the steps of generating at least two different heart tissues in vitro, wherein the different heart tissues are selected from left ventricle progenitor first heart field tissue, right ventricle/outflow tract progenitor second heart field tissue, atrial progenitor second heart field tissue, atrioventricular canal progenitor second heart field tissue, sinoatrial node progenitor second heart field tissue, and atrioventricular node tissue, and fusing the at least two heart tissues, culturing the fused tissue model and letting a calcium signaling connection and/or ability to propagate a tissue contraction and/or a central chamber between the different heart tissues form.

The invention further provides a heart tissue model selected from a right ventricle tissue model comprising cells expressing expression markers IRX1, IRX2 and PRDX1; an atrial tissue model comprising cells expressing expression markers NR2F1, NR2F2 and HEY1; an outflow tract tissue model comprising cells expressing expression markers WNT5A, MSX1, BMP4 and RSPO3; an atrioventricular canal tissue model comprising cells expressing expression markers TBX2, MSX2 and RSPO3; a sinoatrial node tissue model comprising cells expressing expression markers SHOX2, TBX3, HCN4, ISL1 and GJC1; an atrioventricular node tissue model comprising cells expressing expression markers TBX3, TBX5, KCNE1, HCN4 and GJC1.

The invention further provides a method for screening or testing a candidate compound on its effects on heart development and/or functionality comprising generating a heart tissue model according to the invention while treating the cells with the candidate compound and comparing development of the heart tissue model with development and/or or functionality of a heart tissue model that was not treated with the candidate compound.

Further provided is a method of observing the effects of suppressed, mutated or overexpressed genes during heart development comprising generating a heart tissue model according to the invention wherein the cells have a suppressed or mutated candidate gene or overexpress a candidate gene and comparing development of the heart tissue model with development of a heart tissue model that was not generated with a suppressed, mutated or overexpressed gene.

Further provided is a method of screening or testing a candidate compound on its effects on heart functionality comprising treating any heart tissue model of the invention with the candidate compound and comparing with a functionality of a heart tissue model that was not treated with the candidate compound.

The invention further provides a method of treating a heart injury in a patient comprising transplanting a cell from a heart tissue model of the invention to the injury.

The invention further provides a cell culture medium useful in any one of the inventive methods or particular method steps. Such media may be combined in a kit of different media or a kit of one or more medium and another means used in the inventive method, e.g. a carrier or mold used for fusion of cultured tissues.

All embodiments of the invention are described together in the following detailed description and all preferred embodiments relate to all embodiments, aspects, methods, heart tissue models, organoids, uses, media and kits alike. E.g. media and kits or their components can be used in or be suitable for inventive methods. Any component used in the described methods can be part of the medium or kit. Inventive tissue models or organoids are the results of inventive methods or can be used in inventive methods and uses. Preferred and detailed descriptions of the inventive methods read alike on suitability of resulting or used organoids or tissue models of the invention. All embodiments can be combined with each other, except where otherwise stated.

### DETAILED DESCRIPTION

The invention provides a heart tissue model comprising a heart tissue with at least one inner cavity or a central chamber, wherein the heart tissue model comprises at least two different heart tissues selected from left ventricle tissue, right ventricle tissue, atrial tissue, outflow tract tissue, atrioventricular canal tissue, sinoatrial node tissue, and atrioventricular node tissue, wherein the central chamber can be shared by at least two different heart tissues, and/or wherein the at least two different heart tissues comprise a calcium signaling connection and/or ability to propagate a tissue contraction. Also provided is a heart tissue model comprising a heart tissue with a central chamber, wherein the central chamber is shared by at least two different heart tissues selected from left ventricle tissue, right ventricle tissue, atrial tissue, outflow tract tissue, atrioventricular canal tissue, sinoatrial node tissue, and atrioventricular node tissue. The inventive tissue model can be generated from mesoderm cells (which in turn can be generated from pluripotent cells, such as induced pluripotent cells or embryonic stem cells). The tissue model comprises an inner cavity in at least one of the different heart tissues. It is possible that the inner cavity extends to at least another one of the different heart tissues, e.g. it is directly surrounded by tissue from at least two of the different heart tissues. Such an inner cavity is then referred to as a central chamber. The central chamber can form from tissue fusions of at least two different heart tissues selected from left ventricle tissue, right ventricle tissue, atrial tissue, outflow tract tissue, atrioventricular canal tissue, sinoatrial node tissue, and atrioventricular node tissue or their tissue precursors, selected from left ventricle progenitor first heart field tissue, right ventricle/outflow tract progenitor anterior second heart field tissue, atrial progenitor posterior second heart field tissue, outflow tract progenitor anterior second heart field tissue, atrioventricular canal progenitor posterior second heart field tissue and sinoatrial node progenitor posterior second heart field tissue. Atrioventricular canal progenitor second heart field tissue is a progenitor tissue for atrioventricular canal tissue and atrioventricular node tissue. Usually, the central chamber forms after fusion. It may start as an inner cavity in one of the different tissues or precursors and then extend into another one or more of the different heart tissues. A precursor stage of the central chamber, e.g. small tissue bubbles or an inner cavity, may already exist in the different heart tissues before fusion. The different heart tissues or the tissue precursors are each also aspects of the present invention.

The inventive tissue model preferably contains at least two different heart tissues that comprise an electrophysiological signaling connection, e.g. in particular a calcium signalling connection. An electrophysiological signaling connection means that the different heart tissues are functionally connected and can communicate through a calcium/voltage signaling event via intercellular connections. Such an electrophysiological signaling connection may be by gap junctions and/or ion channels between cells of the different heart tissues. Furthermore, tight junctions and/or desmosomes may be present for an electrophysiological signaling connection as they maintain structural connections of the cells and facilitate or improve signal propagation. Electrophysiological signalling can be tracked through either calcium or voltage signal propagation. A calcium signal may be release of calcium from the ER into the cytoplasm in a cell. Such a signal can be propagated to neighbouring cells. The calcium signaling connection may be observed by a dye or a reporter line which tracks calcium or voltage signal propagation. The dye may be an intracellular calcium sensitive dye or a voltage sensitive dye (e.g. FluoroVolt). The dye may get into the cell through expressing a calcium or voltage sensitive dye as a protein or transgene. Alternatively, the dye may be introduced into a cell through a cell culture medium. A further way to measure a signaling event and the voltage signaling connection between different heart tissues may be detected with a multi-electrode array.

The inventive tissue model may comprise at least two different heart tissues with the ability to propagate a contraction. The contraction is a tissue contraction, with the cells of the tissue being able to perform the contraction action. Usually stimulation, e.g. by a cell action potential and/or calcium signaling, can cause the cells to contract. A contraction propagation means that a contraction of one of the heart tissues may propagate into another one of the different heart tissues, usually a neighbouring heart tissue. Contraction may be stimulated by the neighbouring heart tissues contraction but usually though the calcium signaling connection. So, these embodiments may be combined, i.e. a heart tissue model may comprise calcium signaling connection and contraction propagation between at least two different heart tissues. A contraction may be observed as a beating behaviour that starts in one of the different tissues and propagates or projects into a neighbouring heart tissue. The neighbouring heart tissue may start contracting or beating in response to the earlier heart tissue's beating or contraction. The response may have a short time delay. Contraction propagation may be a contraction of an inner cavity or of the central chamber. The contraction may be a contraction by cardiomyocytes. Cardiomyocytes are usually the main contracting cells. Other cell types like fibroblasts and endothelial cells may be involved especially in signal propagation for contraction.

The inventive heart tissue model is an advanced tissue model that recapitulates function of particular heart compartments. It is therefore preferably a cardiac organoid, also referred to as cardioid.

“Selected from” means that the selected species can be from any one of the members that are grouped as selectable species.

Related thereto, the invention provides a method to generate the heart tissue model comprising generating at least two different heart tissues in vitro, wherein the different heart tissues are selected from left ventricle progenitor first heart field tissue, right ventricle/outflow tract progenitor anterior second heart field tissue, right ventricle progenitor anterior second heart field tissue, atrial progenitor posterior second heart field tissue, outflow tract progenitor anterior second heart field tissue and atrioventricular canal progenitor posterior second heart field tissue, and fusing the at least two heart tissues, culturing the fused tissue model and letting a calcium signaling connection, ability to propagate a contraction and/or a central chamber between the different heart tissues form. Fusion is a merger of tissues to form one (combined or fused) tissue. Fusion can be facilitated by placing the different heart tissues into proximity to each other, preferably by contacting each other. Fusion can be by or followed by growth and/or migration of cells, thereby merging the at least two different heart tissues. Preferably fusion of the tissues is done during the progenitor stage of the tissue, e.g. as described further herein. Preferably, the connected area between two tissues at a fusion junction is at least 5000 μm² and/or at least 100 μm in length. The fused tissues may be elongated. Preferably the fused tissues are able to pass calcium and voltage signal from one tissue to another. Right ventricle progenitor (anterior) second heart field tissue and outflow tract (anterior) second heart field tissue is also referred to as right ventri-cle/outflow tract progenitor anterior second heart field tissue. It is one progenitor tissue of the (anterior) second heart field tissue that can differentiate into right ventricle tissue and outflow tract tissue.

To generate a controlled in vitro system of human heart development, the in vivo principles governing the coalescence of all lineages building a heart are used. Most heart structures are derived from three progenitor populations giving rise to specific cardiomyocyte (CM) lineages. The first heart field (FHF) progenitors give rise to the developing left ventricle (LV) tissue, the anterior second heart field (aSHF) gives rise to the developing right ventricle (RV) tissue and most of the outflow tract tissue (OFT), and the posterior second heart field (pSHF) gives rise to most of the atria, a portion of the atrioventricular canal tissue (AVC) the atrioventricular node tissue (AVN) and sinoatrial node tissue (SAN). The development of these structures is time-dependent; for instance, the FHF-derived CMs form the heart tube that then grows into the LV, while the aSHF and pSHF progenitor differentiate and form the other compartments in a delayed and gradual fashion. This process is orchestrated by developmental signaling through multiple pathways (WNT, Activin/Nodal, BMP, RA, FGF, NOTCH, etc.) at specific stages of cardiogenesis. These signaling pathways control down-stream key compartment-specific TFs (TBX1, TBX5, FOXF1, TBX3, ISL1, IRX4, HEY1/2, etc.), instructing progenitor specification, morphogenesis and physiology at specific stages. Some of these signals are generated by the developing tissues or their environment. Some signaling factors may be supplied to a developing tissue to steer development down a developmental route or to create artificial alterations of development.

Preferably, the heart tissue model and the different heart tissues are mammalian tissues, preferably human or non-human primate tissues; likewise, preferably the heart tissue model is a mammalian or human or non-human primate tissue culture, e.g. a mammalian, human or non-human primate cell aggregate. The different heart tissues may stem from culturing mammalian, human or non-human primate cells, such as pluripotent cells, such as induced pluripotent cells (iPS cells) or embryonic stem cells. Human cells, tissues and cultures are especially preferred.

A “pluripotent” cell is not capable of growing into an entire organism, but is capable of giving rise to cell types originating from all three germ layers, i.e., mesoderm, endoderm, and ectoderm, and may be capable of giving rise to all cell types of an organism. Pluripotency can be a feature of the cell per se, e.g. in embryonic stem cells, or it can be induced artificially. E.g. in a preferred embodiment of the invention, the pluripotent stem cell is derived from a somatic, multipotent, unipotent or progenitor cell, wherein pluripotency is induced.

Such a cell is referred to as an induced pluripotent stem (iPS) cell herein. The somatic, multipotent, unipotent or progenitor cell can e.g. be from a patient, which is turned into a pluripotent cell, that is subject to the inventive methods. Such a cell or the resulting tissue culture can be studied for abnormalities, e.g. during heart tissue development according to the inventive methods. A patient may e.g. suffer from a cardiac disorder or heart tissue deformity. Characteristics of said disorder or deformity can be reproduced in the inventive tissue cultures and investigated.

A “multipotent” cell is capable of giving rise to at least one cell type from each of two or more different organs or tissues of an organism, wherein the said cell types may originate from the same or from different germ layers, but is not capable of giving rise to all cell types of an organism. An example of multipotent cells are mesoderm cells.

In contrast, a “unipotent” cell is capable of differentiating to cells of only one cell lineage.

A “progenitor cell” is a cell that, like a stem cell, has the ability to differentiate into a specific type of cell, with limited options to differentiate, with usually only one target cell. A progenitor cell is usually a unipotent cell, it may also be a multipotent cell.

Similar to a progenitor cell, a “progenitor tissue” or “precursor tissue” contains differentiated cells that determines the tissues developmental fate, if left undisturbed. Examples are left ventricle progenitor first heart field tissue, which is a first heart field (FHF) tissue that is destined to develop into a left ventricle (LV) tissue; right ventricle/outflow tract progenitor second heart field tissue, which is a second heart field (SHF) tissue, in particular anterior second heart field (aSHF) tissue, that is destined to develop into right ventricle (RV) tissue or outflow tract (OFT) tissue; right ventricle progenitor second heart field tissue, which is a second heart field (SHF) tissue, in particular anterior second heart field (aSHF) tissue, that is destined to develop into right ventricle (RV) tissue; atrial progenitor second heart field tissue, which is a second heart field (SHF) tissue, in particular posterior second heart field (pSHF) tissue, that is destined to develop into atrial tissue; outflow tract progenitor second heart field tissue, which is a second heart field (SHF) tissue, in particular anterior second heart field (aSHF) tissue, that is destined to develop into outflow tract (OFT) tissue; and atrioventricular canal progenitor second heart field tissue, which comprises a second heart field (SHF) tissue, in particular posterior second heart field (pSHF) tissue, that is destined to develop into atrioventricular canal (AVC) tissue or atrioventricular node (AVN) tissue; and sinoatrial node progenitor second heart field tissue, which comprises a second heart field (SHF) tissue, in particular posterior second heart field (pSHF) tissue, that is destined to develop into sinoatrial node (SAN) tissue.

Gene names or gene symbols as used herein refer to the human genes and are described in databases such as GeneCards (www.genecards.org) or the HGNC database (www.genenames.org). Gene symbols are defined e.g. by the “HUGO Gene Nomenclature Committee” (HGNC). Other designations, such as long names, can be found at their website.

In the inventive heart tissue model or the different heart tissues, preferably the left ventricle tissue or the left ventricle progenitor first heart field comprises at least 60% cardiac cells selected from cardiomyocytes, endocardial cells and epicardial cells. Such a tissue has been described previously (Hofbauer et al., Cell 2021; 184 (12): 3299-3317. e22; and WO 2021/186044 A1; both incorporated herein by reference) and can be used for the present invention. Preferably at least 70% or at least 80% of the cells of the left ventricle tissue or the left ventricle progenitor first heart field are cardiac cells selected from cardiomyocytes, endocardial cells and epicardial cells. Another term for endocardial cells is cardiac endothelial cells.

In preferred embodiments, the right ventricle tissue and/or the right ventricle progenitor second heart field tissue comprises at least 60% cardiomyocytes. Preferably the content of cardiomyocytes in these tissues is at least 70%, especially preferred at least 80%.

In preferred embodiments, the atrial tissue and/or the atrial progenitor second heart field tissue comprises at least 60% cardiomyocytes. Preferably the content of cardiomyocytes in these tissues is at least 70%, especially preferred at least 80%.

In preferred embodiments, the outflow tract tissue and/or the outflow tract progenitor second heart field tissue comprises at least 60% cardiomyocytes. Preferably the content of cardiomyocytes in these tissues is at least 70%, especially preferred at least 80%.

In preferred embodiments, the right ventricle tissue and/or the right ventricle/outflow tract progenitor second heart field tissue comprises at least 60% cardiomyocytes. Preferably the content of cardiomyocytes in these tissues is at least 70%, especially preferred at least 80%.

In preferred embodiments, the atrioventricular canal tissue and/or the atrioventricular canal progenitor second heart field tissue comprises at least 60% cardiomyocytes. Preferably the content of cardiomyocytes in these tissues is at least 70%, especially preferred at least 80%.

In preferred embodiments, the atrioventricular node tissue comprises at least 60% cardiomyocytes. Preferably the content of cardiomyocytes in these tissues is at least 70%, especially preferred at least 80%.

In preferred embodiments, the sinoatrial node tissue and/or the sinoatrial node progenitor second heart field tissue comprises at least 60% cardiomyocytes. Preferably the content of cardiomyocytes in these tissues is at least 70%, especially preferred at least 80%. In later development of the inventive heart tissue model, the number of other cardiac cells, including endocardial cells and epicardial cells in addition to cardiomyocytes, may increase, also in these tissues. Thus, the invention also contemplates that any of the right ventricle tissue, the atrial tissue, the outflow tract tissue, and the atrioventricular canal tissue comprises at least 60% cardiac cells selected from cardiomyocytes, endocardial cells and epicardial cells. Preferably at least 70% or at least 80% of the cells of these tissues are cardiac cells selected from cardiomyocytes, endocardial cells and epicardial cells.

Of course, any of these cell numbers can be combined for the respective tissues, when present, in the inventive heart tissue model. The heart tissue model itself may comprises at least 60% cardiac cells selected from cardiomyocytes, endocardial cells and epicardial cells, especially if left ventricle tissue is present. Left ventricle tissue, when present, is usually a large part of the heart tissue model. Preferably at least 70% or at least 80% of the cells of the heart tissue model are cardiac cells selected from cardiomyocytes, endocardial cells and epicardial cells. In combinable embodiments with these numbers, at least 40%, preferably at least 50%, especially preferred at least 60% or at least 708, of the cells of the heart tissue model are cardiomyocytes.

The inventive heart tissue model comprises at least one inner cavity or one large chamber, the central chamber, which simulates a heart chamber of a natural heart. If functional, e.g. in a healthy condition without disturbing mutations or chemicals, a beating rhythm with variation in the volume distribution of the inner cavity or central chamber can be observed. Since the heart tissue model is still artificial and not connected to a blood circulation system as pump therein, it usually lacks any large blood vessel into (and out of) the inner cavity or central chamber. Preferably the inner cavity or central chamber is completely surrounded by the different heart tissues, in particular the tissue selected from left ventricle tissue, right ventricle tissue, atrial tissue, outflow tract tissue or atrioventricular canal tissue. Alternatively or in combination therewith the volume of the inner cavity or central chamber is not leading into a major blood vessel and/or is not developing a blood vessel. It is possibly to artificially, e.g. surgically graft a blood vessel to the heart tissue model but such a blood vessel would not naturally form from the fusion product of the invention without artificial intervention. A “major blood vessel” is a blood vessel that resembles one of the great vessels of a mammalian heart (venae cavae and pulmonary veins; aorta and pulmonary arteries). Such a major blood vessel may have a diameter of 10% or more of the diameter of one of the different heart tissues forming the heart tissue model. Reference to one of the different heart tissues is made since such a major blood vessel would enter the inner cavity or central chamber usually at one of the tissues. Preferably, the volume of the inner cavity or central chamber is not leading into a blood vessel.

The inventive tissue model is artificial and grown in culture using natural principles of development, but not an in vivo grown heart at any of an in vivo heart's development. Due to the culture process, usually the size is limited. In preferred embodiments of the invention, the heart tissue model has a size in its largest dimension of 0.3 mm to 50 mm, even more preferred 1 mm to 40 mm or especially preferred 2 mm to 30 mm. Due to the fusion of different heart tissues, the heart tissue model may have an elongated shape. For reference the largest dimension of that shape is used to determine the heart tissue model's size.

The inner cavity or central chamber is a cavity within the heart tissue model and is quite large (as compared to previous heart tissue models) and takes up a major part of the tissue model's volume. Preferably, the size of the inner cavity or central chamber at its largest dimension is at least 30% of the size of the heart tissue model at its largest dimension. If there are more cavities besides the inner cavity or central chamber, this applies only to the largest inner cavity or central chamber (and the surrounding tissue, not extending to tissue layers that surround another inner cavity). In preferred embodiments, the size of the inner cavity or central chamber at its largest dimension is at least 40%, more preferred at least 50%, even more preferred at least 608, of the size of the heart tissue model at its largest dimension. Both the central chamber and the heart tissue model may be of an elongated shape due to the fusion of different heart tissues and the extension of a central chamber into the different heart tissues. Similar as above, the largest dimension within that shape is used.

In some embodiments (e.g. multi-cavity cardioids), at least two, e.g. three or more, of the different heart tissues has an inner cavity. The inner cavities of the different heart tissues may be as described above, e.g. having a size of at least 30% of the size of the heart tissue model. Any of the inner cavities may also be smaller, especially when confined to a single different heart tissue. An inner cavity may have a size at its largest dimension of at least 20% of the size of the heart tissue model at its largest dimension. Also, an inner cavity may have a size at its largest dimension of at least 30%, preferably at least 40%, of the size of the heart tissue at its largest dimension. The heart tissue here only takes account of the part of the tissue model that belongs to one particular heart tissue that confines the inner cavity.

Cardiomyocytes are cardiac muscle cells and are mostly re-sponsible for the beating activity or tissue contraction of the heart tissue model or the different heart tissues. They are the main component of the inventive tissue model and form a layer in the tissue model surrounding the inner cavity or central chamber. In early development tissues, they may directly face the inner cavity or central chamber but in later and more developed tissues the inner layer is formed by endocardial cells. Accordingly, in preferred embodiments cardiomyocytes or endocardial cells directly face the inner cavity or central chamber of the heart tissue model and/or of the different heart tissues, especially preferred the heart tissues selected from left ventricle tissue, right ventricle tissue, atrial tissue, outflow tract tissue, atrioventricular canal tissue, sinoatrial node tissue, and atrioventricular node tissue.

The heart tissue model and its different heart tissues but also the different heart tissues themselves (before fusion), which form a further aspect of the invention, and the precursor tissues, including FHF, aSHF, pSHF, or the multipotent or pluripotent stem cell, may be characterized by particular expression patterns or expression markers. These expression patterns are of the cells that constitute the heart tissues/tissue model/precursors. Gene names or gene symbols (see above) are used to characterize the expression markers. Such patterns are shown in the figures. The expression of cell markers may be determined by any suitable technique, including immunocytochemistry, immunofluorescence, RT-PCR, immunoblotting, fluorescence-activated cell sorting (FACS), RNA sequencing, single cell RNA-sequencing and enzymatic analysis. In the following some characteristic expression markers are given. Some markers may be over-expressed or underexpressed, in some cases below a detection limit. Preferably overexpression is an elevated expression in comparison with expression in a human embryonic stem cell. Preferably underexpression is a reduced expression in comparison with expression in a human embryonic stem cell. Underexpression may also constitute no detectable expression.

The term “expresses an expression marker” means that expression of mRNA encoding a marker is detectable above background levels using RT-PCR or RNA sequencing or single cell RNA-sequencing, preferably RT-PCR. The expression level of an expression marker can be compared to the expression level obtained from a negative control (i.e., cells known to lack the marker) or by isotype controls (i.e., a control antibody that has no relevant specificity and only binds non-specifically to cell proteins, lipids or carbohydrates). Thus, a cell that “ex-presses” a marker has an expression level detectable above the expression level determined for the negative control for that marker. Alternatively, cell surface markers may be detectable above background levels on the cell using immunofluorescence microscopy or flow cytometry methods, such as fluorescence activated cell sorting (FACS).

The terms “lacks expression”, “does not express” and “absent marker” mean that expression of the mRNA for an expression marker cannot be detected above background levels using RT-PCR or RNA sequencing or single cell RNA-sequencing, preferably RT-PCR. The expression level of an expression marker can be com-pared to the expression level obtained from a negative control (i.e., cells known to lack the marker) or by isotype controls (i. e., a control antibody that has no relevant specificity and only binds non-specifically to cell proteins, lipids or carbohydrates). Thus, a cell that “lacks expression” of a marker ap-pears similar to the negative control with respect to that marker. Alternatively, a cell surface marker may not be detected above background levels on the cell using immunofluorescence microscopy or flow cytometry methods, such as fluorescence activated cell sorting (FACS).

Preferably the left ventricle tissue cells express one or more expression markers selected from NPPA, IRX4 and HEY2; and/or the left ventricle tissue cells lack expression of one or more expression markers selected from NR2F2, TBX2 and TBX3. NR2F2, TBX2 and TBX3 may be absent or underexpressed after maturation of the left ventricle tissue.

Preferably the right ventricle tissue cells express one or more expression markers selected from NPPA, IRX1, IRX2 and PRDX1; and/or the right ventricle tissue cells lack expression of one or more expression markers selected from NR2F2, TBX2 and WNT5A. NR2F2, TBX2 and WNT5A may be absent or underexpressed after maturation of the right ventricle tissue.

Preferably the atrial tissue cells express one or more ex-pression markers selected from NPPA, NR2F1, NR2F2 and HEY1; and/or the atrial tissue cells lack expression of one or more expression markers selected from IRX1, IRX4 and HEY2. IRX1, IRX4 and HEY2 may be absent or underexpressed after maturation of the atrial tissue.

Preferably the outflow tract tissue cells express one or more expression markers selected from WNT5A, MSX1, BMP4, WNT11 and RSPO3; and/or the outflow tract tissue cells lack expression of one or more expression markers selected from TBX3, NR2F1 and NPPA. TBX3, NR2F1 and NPPA may be absent or underexpressed after maturation of the outflow tract tissue.

Preferably the atrioventricular canal tissue cells express one or more expression markers selected from TBX2, MSX2 and RSPO3; and/or the atrioventricular canal tissue cells lack ex-pression of one or more expression markers selected from IRX1, IRX4 and NPPA. IRX1, IRX4 and NPPA may be absent or underexpressed after maturation of the atrioventricular canal tissue.

Preferably the sinoatrial node tissue cells express one or more expression markers selected from SHOX2, TBX3, HCN4, ISL1 and GJC1; and/or the sinoatrial node tissue cells lack expression of one or more expression markers selected from NKX2.5, IRX1, IRX4 and NPPA. NKX2.5, IRX1, IRX4 and NPPA may be absent or underexpressed after maturation of the sinoatrial node tissue.

Preferably the atrioventricular node tissue cells express one or more expression markers selected from TBX3, TBX5, KCNE1, HCN4 and GJC1; and/or the sinoatrial node tissue cells lack ex-pression of one or more expression markers selected from RSPO3, MSX2, IRX4 and NPPA. RSPO3, MSX2, IRX4 and NPPA may be absent or underexpressed after maturation of the atrioventricular node tissue.

In a further aspect, the invention provides a method to generate a heart tissue model of the invention. The method comprises generating at least two different heart tissues in vitro, wherein the different heart tissues are selected from left ventricle progenitor first heart field tissue, right ventricle/outflow tract progenitor second heart field tissue, atrial progenitor second heart field tissue, outflow tract progenitor second heart field tissue, atrioventricular canal progenitor second heart field tissue, sinoatrial node progenitor second heart field tissue, and atrioventricular node tissue. These at least two different heart tissues may be provided as pre-grown tissues, e.g. in a frozen state. The method then uses the thawed and viable heart tissues. The method further contains the step of fusing the at least two heart tissues; culturing the fused tissue model and letting a calcium signaling connection, tissue contraction propagation behaviour and/or a central chamber between the different heart tissues form.

The invention also provides a heart tissue model obtainable by any method of the invention. The obtained heart tissue model may have any of the structural elements as described above.

Fusing the at least two heart tissues may comprise placing the different heart tissues in contact to each other and letting the tissues fuse. Fusion may comprise cell growth and/or cell migration. Fusion may be achieved by placing two or more heart tissues, preferably at the progenitor stage for the left ventri-cle tissue, right ventricle tissue, atrial tissue, outflow tract tissue, or the later stage for the pacemaker cells (sinoatrial node tissue, and atrioventricular node tissue), into close proximity to each other, preferably into a plate. Usually within a day post fusion, tissues form a structural connection to each other, e.g. the connected area mentioned above. A few days later the structural connection leads to a functional interaction. Preferably, when more than two heart tissues are being fused, fusion molds in an elongated shape are used to have control over the arrangement of the heart tissues. The viable cells of the different heart tissues facilitate the fusion of the tissues to form the heart tissue model. Further growth leads to the formation of the central chamber, once fused. For fusion, the at least two different heart tissues preferably do not yet contain a major central chamber or inner cavity, since this would impair fusion efficiency, and later, central chamber formation spanning more than one of the at least two different heart tissues—with the exception of the left ventricle progenitor first heart field tissue, which may comprise an inner cavity or central chamber at the time of fusion and still be capable of efficient fusion and inner cavity or central chamber formation that ex-tends within the other of the two heart tissues. One or more of the different heart tissues may comprise an inner cavity when placed for fusion or during fusion.

The calcium signaling connection and/or contraction propagation may develop between the different heart tissues after fusion.

It is preferred to grow the at least two different heart tissues in situ before fusion since developmental stages can be better controlled. Some developmental stages are described by culture day. These culture days refer to continued growth in culture at optimal temperature (for the given cell, e.g. 37° C. for human cells) and in growth medium. Freezing of the tissues or cells from which the tissues stem interrupts counting of culture days. Accordingly, “culture days” refers to the time corresponding to culturing in optimal growth conditions for the given cell type.

In preferred embodiments, the different heart tissues have been cultured and differentiated from a pluripotent cell. I.e. a pluripotent cell is grown and differentiated to give rise to the different heart tissues. Starting from culturing the pluripotent cells, the fusion is preferably at culture day 1 to 7 from a pluripotent stage.

In preferred embodiments, right ventricle/outflow tract progenitor second heart field tissue, atrial progenitor second heart field tissue, atrioventricular progenitor second heart field canal tissue, is fused at culture day 2 to 5, again counting from the pluripotent stage. Preferably sinoatrial node progenitor second heart field tissue, and/or atrioventricular node tissue are fused at culture day 6-10, especially preferred to tissues of a similar age. Alternatively, or in combination optimal fusion times are determined by certain expression markers of the tissues. Preferably left ventricle progenitor first heart field tissue is fused when expressing the expression marker TBX5 and/or HAND1. Preferably right ventricle/outflow tract progenitor second heart field tissue is fused when expressing the ex-pression marker TBX1, FOXC1 and/or FOXC2. Preferably atrial tissue is fused when expressing the expression marker HOXB1, TBX5 and/or OSR1. Preferably, atrioventricular canal progenitor second heart field tissue is fused when expressing the expression marker TBX3, FOXF1 and/or HOXB1. Preferably sinoatrial node progenitor second heart field tissue is fused when expressing the expression marker SHOX2, TBX3, HCN4, ISL1 and/or GJC1. Preferably atrioventricular node tissue is fused when expressing the expression marker TBX3, TBX5, KCNE1, HCN4 and/or GJC1. These markers indicate development to a condition suitable for efficient fusion.

Generating the different tissues can be done using media and/or growth factors and/or differentiation factors. Examples are given in the example section herein, especially examples 1 and 2. Any of the differentiation factors mentioned there can be used for the inventive method in any combination and in isolation or together with other differentiation markers that achieve the same differentiation development. Some preferred components for culturing are described in the following for particular tissues.

In particular preferred embodiments, one of the at least two different heart tissues is left ventricle progenitor first heart field tissue. Generating left ventricle progenitor first heart field tissue may comprise differentiating mesoderm cells into left ventricular precursor cells in a medium comprising a bone morphogenic protein, preferably BMP4, a fibroblast growth fac-tor, preferably FGF2, insulin, a Wnt inhibitor, preferably Wnt-C59 or IWP2, and retinoic acid, with the retinoic acid having a concentration of 5 nM to 100 nM, in the medium. Culturing with this medium is preferably done at about culture day 2, e.g. during culture day 1.5 to 3.

Especially preferred, one of the at least two different heart tissues is right ventricle progenitor anterior second heart field tissue or right ventricle/outflow tract progenitor anterior second heart field tissue. Generating right ventricle progenitor anterior second heart field tissue or right ventri-cle/outflow tract progenitor anterior second heart field tissue may comprise differentiating mesoderm cells into right ventricular precursor cells in a medium comprising a TGF-beta inhibitor, preferably SB 431542, and a Wnt inhibitor, preferably Wnt-C59 or XAV-939. Culturing with this medium is preferably done at about culture day 2, e.g. during culture day 1.5 to 3. Generating right ventricle progenitor anterior second heart field tissue and especially further developing to right ventricle tissue may further comprise cultivation in a medium comprising a Wnt inhibitor, preferably Wnt-C59 or XAV-939, a bone morphogenic protein, preferably BMP4, a fibroblast growth factor, preferably FGF2, insulin, and retinoic acid, preferably with the retinoic acid having a concentration of 50 nM to 500 nM in the medium.

Preferably one of the at least two different heart tissues is outflow tract progenitor anterior second heart field tissue. Generating outflow tract progenitor anterior second heart field tissue may comprise differentiating mesoderm cells into outflow tract tissue precursor cells in a medium comprising a TGF-beta inhibitor, preferably SB 431542, and a Wnt inhibitor, preferably Wnt-C59 or XAV-939. Culturing with this medium is preferably done at about culture day 2, e.g. during culture day 1.5 to 3. Generating outflow tract progenitor anterior second heart field tissue or further to outflow tract tissue may further comprise cultivation in a medium comprising a Wnt inhibitor, preferably Wnt-C59 or XAV-939, a bone morphogenic protein, preferably BMP4, a fibroblast growth factor, preferably FGF2, insulin, and lacking retinoic acid.

Preferably one of the at least two different heart tissues is atrial progenitor posterior second heart field tissue. Generating atrial progenitor posterior second heart field tissue may comprise differentiating mesoderm cells into atrial tissue precursor cells in a medium comprising a TGF-beta inhibitor, preferably SB 431542, a Wnt inhibitor, preferably Wnt-C59 or XAV-939, and retinoic acid, preferably the retinoic acid is in a concentration of 300 nM to 800 nM. Culturing with this medium is preferably done at about culture day 2, e.g. during culture day 1.5 to 3. Generating atrial progenitor posterior second heart field tissue or further differentiation to atrial progenitor tissue may comprise further cultivation in a medium comprising a Wnt inhibitor, preferably Wnt-C59 or XAV-939, a bone morphogenic protein, preferably BMP4, a fibroblast growth factor, preferably FGF2, insulin, and retinoic acid, preferably the retinoic acid is In a concentration of 300 nM to 800 nM.

Preferably one of the at least two different heart tissues is atrioventricular canal progenitor posterior second heart field tissue. Generating atrioventricular canal progenitor posterior second heart field tissue may comprise differentiating mesoderm cells into atrioventricular canal tissue precursor cells in a medium comprising a TGF-beta inhibitor, preferably SB 431542, a Wnt inhibitor, preferably Wnt-C59 or XAV-939, a bone morphogenic protein, preferably BMP4 and retinoic acid, preferably the retinoic acid is in a concentration of 300 nM to 800 nM. Culturing with this medium is preferably done at about culture day 2, e.g. during culture day 1.5 to 3. Generating atrioventricular canal progenitor posterior second heart field tissue or further differentiation to atrioventricular canal tissue may comprise further cultivation in a medium comprising a Wnt inhibitor, preferably Wnt-C59 or XAV-939, a bone morphogenic protein, preferably BMP4, a fibroblast growth factor, preferably FGF2, insulin, and retinoic acid, preferably the retinoic acid is in a concentration of 300 nM to 800 nM.

Preferably one of the at least two different heart tissues is sinoatrial node progenitor posterior second heart field tissue and generating sinoatrial node progenitor posterior second heart field tissue comprises differentiating mesoderm cells into sinoatrial node tissue precursor cells in a medium comprising a TGF-beta inhibitor, preferably SB 431542, a bone morphogenic protein, preferably BMP4 and retinoic acid, preferably the retinoic acid is in a concentration of 300 nM to 800 nM.

Preferably one of the at least two different heart tissues is atrioventricular node tissue and generating atrioventricular node tissue comprises differentiating mesoderm cells into atrioventricular canal tissue precursor cells in a medium comprising a TGF-beta inhibitor, preferably SB 431542, a Wnt inhibitor, preferably Wnt-C59 or XAV-939, a bone morphogenic protein, preferably BMP4 and retinoic acid, preferably the retinoic acid is in a concentration of 300 nM to 800 nM. Generating atrioventricular node tissue may further comprise differentiating atrioventricular canal progenitor posterior second heart field tissue into atrioventricular node tissue by further cultivation in a medium containing an activator or inhibitor of sonic hedgehog signaling and/or a bone morphogenic protein.

Fusion of cardiac tissues is under culture conditions that allow fusion and does not impede differentiation of the progenitor tissues into their target tissues, during fusion or at a later stage. Preferably fusing the at least two heart tissues comprises culturing in a medium comprising a Wnt inhibitor, preferably Wnt-C59, a bone morphogenic protein, preferably BMP4, a fibroblast growth factor, preferably FGF2, insulin, and retinoic acid, preferably the retinoic acid is in a concentration of 300 nM to 800 nM, especially preferred 500 nM. These are excel-lent culturing conditions for the heart tissues mentioned herein.

Mentioned above are mesoderm cells as progenitors for the inventive tissue differentiation. The mesoderm cell can be de-rived from a pluripotent cell. Preferably generating at least two different heart tissues in vitro comprises generating meso-derm cells. Mesoderm cells can be generated by differentiating pluripotent cells in a medium comprising activin and a Wnt activator, preferably CHIR99021; preferably at least until differentiated cells express expression markers selected from BRA, EOM, MIXL1, FOXA2, preferably in the absence of SOX2. Preferably the medium for generating mesoderm cells further comprises a PI3 kinase inhibitor, such as LY294002. The use of the PI3 kinase inhibitor is particularly preferred as it results in the cleanest and most homogenous tissue models. A PI3 kinase inhibitor has the effect that many pluripotent cells exit pluripotency and differentiate to mesodermal lineage, which determines the future highly homologous cardiac tissue model. LY294002 is preferably used in a concentration of 3 μM to 15 μM.

Already at the stage of mesoderm cells, a prevalence for differentiation into specific tissues exist. E.g. mesoderm cells can be progenitors of the first heart field (FHF) or second heart field (SHF), including aSHF and pSHF.

For mesoderm cells that are further differentiated into left ventricle tissue (FHF), the activin is preferably at a concentration of 1 ng/mL to 8 ng/ml, more preferred about 5 ng/ml, and/or the CHIR99021 is preferably at a concentration of 1 μM to 6 μM, more preferred about 3 μM. For mesoderm cells that are further differentiated into right ventricle tissue, atrial tissue or outflow tract tissue (a/pSHF) the activin is preferably at a concentration of 30 ng/mL to 100 ng/ml, more preferred about 50 ng/ml, and/or the CHIR99021 is preferably at a concentration of 2 μM to 8 μM, more preferred about 4 μM. For mesoderm cells that are further differentiated into atrioventricular canal tissue (partially pSHF) or atrioventricular node tissue the activin is preferably at a concentration of 6 ng/ml to 30 ng/ml, more preferred about 10 ng/ml, and/or the CHIR99021 is preferably at a concentration of 0.4 μM to 4 μM, more preferred about 2 μM. For mesoderm cells that are further differentiated into sinoatrial node tissue preferably the activin is at a concentration of 30 ng/mL to 100 ng/ml, preferably about 50 ng/ml, and/or the CHIR99021 is at a concentration of 0.1 μM to 4 μM, preferably about 1 μM.

Preferably the pluripotent stem cells have been grown in a medium comprising at least 1.5% (w/v) (at least 1.5 g/l), albumin, preferably BSA, and/or at least 100 ng/ml fibroblast growth factor, preferably FGF2.

Preferably the cultivation of pluripotent stem cells and their differentiation to mesoderm cells is in a 2D culture. Later steps, e.g. the cultivation of mesoderm cells and their differentiation to progenitor cells and the generation of the different heart tissues and the tissue models is in a 3D culture.

A 2D culture may include growing or maintaining the cells on a surface, such as a tissue culture plate. Such a surface may be coated with suitable culturing substrates, such as vitronectin.

In 3D culture, the cells or the aggregates are not attached to a surface but the cells are allowed to aggregate to each other or remain free floating in a suspended condition so that expansion in all 3D directions is uniformly possible. Such culturing is in a low attachment culture so that the cells or aggregates do not attach to culture vessel walls. Preferably, the low attachment culture comprises culturing the cells in a container with a low attachment surface that inhibits adherence of the cells to the surface. Low attachment surfaces are preferably hydrophilic, neutrally charged or non-ionic. They may have a hydrogel layer or coating that repels cell attachment, forcing cells into a suspended state. Such low attachment culture vessels are known in the art, e.g. described in WO 2019/014635 or WO 2019/014636. Preferably the bottom of the culturing vessel is round, especially concave. Alternatively, it may be flat or have a V-bottom shape.

Preferably the pluripotent cells may then be dissociated cells for starting the culture. They are then allowed to form aggregates. The mesoderm cells are preferably cultures already in the aggregate state, e.g. as obtained from culturing and differentiating the pluripotent cells. The aggregates then lead to the formation of the different tissues and tissue models as enlargement of the aggregates.

Preferably a cell culture medium is used, such as E8 medium. A cell culture medium used in the course of the invention preferably comprises amino acids required for cell growth and an energy source, such as a carbohydrate, especially preferred glucose. It further shall comprise salts and ions needed for cell growth, such as Ca, Fe, Mg, K, Na, Zn, Cl, SO₄, NO₃, PO₄ ions. Further preferred components of the medium are vitamins, such as vitamin B-12, Biotin, choline, folic acid, inositol, niacinamide, pantothenic acid, pyridoxine, riboflavin, thiamine.

Preferred amino acids include essential amino acids, prefer-ably also any one of the following amino acids alanine, argi-nine, asparagine, aspartic acid, cysteine, cystine, glutamic acid, glutamine, glycine, histidine, isoleucine, leucine, lysine, methionine, phenylalanine, proline, serine, threonine, tryptophan, tyrosine, valine.

In preferred embodiments, the cells have been cultured or treated in/with such a medium, especially one comprising albumin and/or a fibroblast growth factor, for at least 1 day, preferably at least 2 days, e.g. 1 to 30 days, such as 2 to 20 days—while maintaining pluripotency of course.

A preferred bone morphogenetic protein (BMP) is BMP4, in particular human BMP4. Preferably a medium with a BMP comprises at least 8 ng/ml bone morphogenetic protein, preferably BMP4.

Some media or culturing contains a FGF (fibroblast growth factor), preferably FGF2, FGF4, FGF8, FGF17, FGF18. Especially preferred is FGF2. FGF2 is preferably used at a concentration of at least 6 ng/ml unless otherwise noted.

WNT inhibitors are known in the art. WNT inhibitors are dis-closed e.g. in Nusse and Clevers, Cell 169, 2017:985-999. Further WNT activators are disclosed at the website web. stan-ford. edu/group/nusselab/cgi-bin/wnt/smallmolecules. The WNT inhibitor is preferably selected from Wnt-C59, IWR-1, XAV939, IWP-2, IWP-4, DKK1 or a combination thereof. Particular preferred WNT inhibitors are IWP-2 and XAV.

In any medium with a Wnt inhibitor or activator, preferably albumin is present. Albumin may be bovine serum albumin (BSA). Albumin protects cells from toxicity of small molecules such as the WNT activators/inhibitors and is therefore recommendable.

The TGF-beta inhibitor may be a TGF-beta signaling pathway inhibitor as it inhibits TGF-beta function. It may be an inhibitor of the TGF-beta superfamily pathways. Preferably one or—more preferred—two, or even more, TGF-beta inhibitors are used. In some preferred embodiments it is comprised of or comprises one or more inhibitors of the TGF-beta receptor or a TGF-beta pathway. In preferred embodiments the at least one TGF-beta (pathway) inhibitor is at least one SMAD inhibitor, preferably DMH1 (dorsomorphin homolog 1)) and/or SB431542 (4-[4-(2H-1,3-Benzodioxol-5-yl)-5-(pyridin-2-yl)-1H-imidazol-2-yl]benzamide). Further preferred TGF-beta inhibitors, that are alternatives or combinable with those mentioned before, are Noggin (a Protein that binds and inactivates BMP proteins, which belong to the TGF-beta superfamily), A 83-01 (a small molecule), LDN 193189 (a small molecule) and/or Dorsomorphine. Further TGF-beta inhibitors are known in the art, e.g. as disclosed at www.med-chemexpress.com/Targets/TGF-(beta)%20Receptor.html.

Activin, e.g. activin A, may be used to specify the heart tissue precursors towards a LV, a/pSHF and SAN, or AVC fate. Generally, high Activin (e.g. higher than 10 ng/ml) steers the tissue model towards an a/pSHF and SAN fate, middle activin (e.g. around 10 ng/ml) steers the tissue model towards an atrioventricular fate, and lower activin (e.g. lower than 10 ng/ml) steers the tissue model towards a left ventricular fate. These example concentrations may vary depending on the cell type used.

Wnt activators are known in the art and described e.g. in Nusse and Clevers, Cell 169, 2017:985-999. Nusse and Clevers discuss the Wnt/b-catenin signaling pathway and its manipulation in stem cells. The Wnt activator may be a GSK3 inhibitor. Further WNT activators are disclosed at the website web.stan-ford.edu/group/nusselab/cgi-bin/wnt/smallmolecules. In preferred embodiments the Wnt activator is a WNT ligand, such as WNT-3a, or CHIR99021 (6-[[2-[[4-(2,4-dichlorophenyl)-5-(5-methyl-1H-imidazol-2-yl)-2-pyrimidinyl]amino]ethyl]amino]-3-pyridinecarbonitrile). Further preferred Wnt activators are WAY-316606, ABC99, IQ1, QS11, SB-216763.

An activator of sonic hedgehog (SHH) signaling is preferably a Smoothened agonist, especially preferred SAG (N-Methyl-N′-(3-pyridinylbenzyl)-N′-(3-chlorobenzo[b]thiophene-2-carbonyl)-1,4-diaminocyclohexane), cyclopamine or purmorphamine (PMA), or a combination thereof. An inhibitor of sonic hedgehog (SHH) signalling is preferably Cyclopamine.

The invention further provides the above-mentioned different heart tissues and their precursors as tissue models.

Thus, in further aspect, provided is a right ventricle tissue model. The right ventricle tissue model may comprise cells expressing expression markers IRX1, IRX2 and PRDX1. The entire disclosure herein for the right ventricle tissue also applies to the right ventricle tissue model.

Further provided is an atrial tissue model. The atrial tissue model may comprise cells expressing expression markers NR2F1, NR2F2 and HEY1. The entire disclosure herein for the atrial tissue also applies to the atrial tissue model.

Further provided is an outflow tract tissue model. The out-flow tract tissue model may comprise cells expressing expression markers WNT5A, WNT11, MSX1, BMP4 and RSPO3. The entire disclosure herein for the outflow tract tissue also applies to the outflow tract tissue model.

Further provided is an atrioventricular canal tissue model. The atrioventricular canal tissue model may comprise cells ex-pressing expression markers TBX2, MSX2 and RSPO3. The entire disclosure herein for the atrioventricular canal tissue also ap-plies to the atrioventricular canal tissue model.

Further provided is a sinoatrial node tissue model. The sinoatrial node tissue model may comprise cells expressing expression markers SHOX2, TBX3, HCN4, ISL1 and GJC1. The entire dis-closure herein for the sinoatrial node tissue also applies to the sinoatrial node tissue model.

Further provided is an atrioventricular node tissue model. The atrioventricular node tissue model may comprise cells ex-pressing expression markers TBX3, TBX5, KCNE1, HCN4 and GJC1. The entire disclosure herein for the atrioventricular node tissue also applies to the atrioventricular node tissue model.

The right ventricle tissue model, atrial tissue model, out-flow tract tissue model, atrioventricular canal tissue model, sinoatrial node tissue model, and atrioventricular node tissue model, collectively also referred to as tissue model, may not be fused or they may be fused to another tissue. The tissue models recapitulate specific parts of the heart and—together with the left ventricle tissue as in WO 2021/186044 A1—are also considered as cardiac organoid, or cardioid.

Any of the tissue models may undergo cavitation and then comprise an inner cavity or central chamber on their own-simi-lar as the fused tissue model as described above. Preferably, the size of the inner cavity or central chamber at its largest dimension is at least 30% of the size of the tissue model at its largest dimension. If there are more cavities besides the central chamber or largest inner cavity, then this applies only to the largest inner cavity or central chamber (and the surrounding tissue, not extending to tissue layers that surround another inner cavity). In preferred embodiments, the size of the inner cavity or central chamber at its largest dimension is at least 40%, more preferred at least 50%, even more preferred at least 60%, of the size of the tissue model at its largest dimension.

The tissue models may comprise calcium signaling or tissue contraction behaviour or the ability for tissue contraction. Calcium signaling and contraction can be observed as mentioned above for the fused heart tissue models-except of course that the single tissue heart models may not show a calcium signaling connection or contraction propagation to another tissue. However similar connections and propagations may be observed between cells of the tissue models.

Preferably the tissue model comprises at least 60% cardiac cells selected from cardiomyocytes, endocardial cells and epicardial cells. Preferably at least 70% or at least 80% of the cells of the tissue model are cardiac cells selected from cardiomyocytes, endocardial cells and epicardial cells. Especially preferred, the tissue model comprises at least 60% cardiomyocytes.

Due to the culture process, usually the size of these tissue models is limited. In preferred embodiments of the invention, the tissue model has a size in its largest dimension of 0.2 mm to 30 mm, even more preferred 0.5 mm to 25 mm or especially preferred 1 mm to 20 mm.

The heart tissue models may be obtained or produced by the methods described above for the different heart tissues, without the fusion to another one of the different heart issues. In particular the heart tissue models may be obtained by the culturing methods described above for the different heart tissues.

The present invention, i.e. the heart tissue model, the method and the different tissue models can be used for screening for effects of candidate compounds and/or effects of genetic modifications or of environmental factors. Such a method may comprise generating a heart tissue model according to the invention while treating the cells (at any stage during development) with a candidate compound. Also, the final tissue model may be used to test the candidate compound.

E.g. the invention provides a method for screening or testing a candidate compound on its effects on heart development and/or functionality comprising generating a heart tissue model according to the method of the invention while treating the cells with the candidate compound and comparing development of the heart tissue model with development and/or or functionality of a heart tissue model that was not treated with the candidate compound.

Further provided is a method of observing the effects of suppressed, mutated or overexpressed genes during heart development comprising generating a heart tissue model according to the invention wherein the cells have a suppressed or mutated candidate gene or overexpress a candidate gene and comparing development of the heart tissue model with development of a heart tissue model that was not generated with a suppressed, mutated or overexpressed gene.

Of course, these methods of screening/testing candidate com-pounds and genetic alterations can be combined. E.g. provided is a method of screening or testing a candidate compound on its effects on heart development and/or functionality according to the invention in combination with observing the effects of sup-pressed or overexpressed genes during heart development according to the invention.

Also provided is a method of screening or testing a candidate compound on its effects on heart functionality comprising treating a heart tissue model according to the invention with the candidate compound and comparing with a functionality of a heart tissue model that was not treated with the candidate com-pound.

The method may comprise comparing development or functionality of the heart tissue model with development or functionality of a heart tissue model that was not treated with the candidate compound. For comparison all treatment steps should be the same except of course the treatment with the candidate compound. An example function is e.g. any heart function, like the beating behaviour (e.g. intensity and/or rhythm, e.g. arrhythmias) or morphological changes or metabolic turnover or genetic expression of genes of interest that may be influenced by the candidate compound. In the inner cavity or central chamber fluid flow and/or electrical flow in the tissue surrounding the inner cavity or central chamber may also be observed. Development of function may be a toxic development or function that may be caused by the candidate compound. As such, the inventive method or tissue model can be used for toxicity testing or screening.

Genetic alteration may be tested. E.g. The invention pro-vides a method of observing the effects of mutated (e.g. dis-ease-related), suppressed, mutated or overexpressed genes during heart development comprising generating a heart tissue model according to the invention wherein the cells have a suppressed or mutated candidate gene or overexpress a candidate gene and com-paring development of the heart tissue model with development of a heart tissue model that was not generated with a suppressed, mutated or overexpressed gene. Here suppression or overexpression is in relation to a normal state, without the modification of the candidate gene's expression or mutation. Mutation, over-expression or suppression of expression can be done with any known method in the art, such as suppression by gene knock out, siRNA inhibition or CRISPR/Cas-based inactivation. Overexpression can be done by introduction of a transgene or applying a gene activator that leads to upregulation of expression of a gene. Such mutated tissues or method using mutation in the cells and generating inventive cardiac tissues with these cells can also be used for screening or testing a candidate compound as discussed above. As such, the invention provides using the inventive cardiac tissues or the methods as disease models, e.g. heart disease models, in particular to study heart functionalities. Accordingly, the method of screening or testing a candidate compound can also be combined with observing the effects of suppressed or overexpressed genes. In the combined method, the comparisons can be made between the variants of the tissue or method with (“drugged”) or without (“not-drugged”) candidate treatment, both with the mutation or between the mutated and non-mutated variants, both in drugged configuration, or a com-parison between all four states (mutated+drugged, mutated+not-drugged, non-mutated+drugged, non-mutated+not-drugged).

The inventive heart tissue may be used to recapitulate specific normal or abnormal conditions of the heart, such as diseases, illnesses or injuries, as well as a recovery from such conditions. Diseases may for example be congenital defects of the heart or myopathies or hypertrophies. Factors that be investigated, e.g. by making the cells or aggregates during the method or the tissue model susceptible or exposed to such fac-tors, are e.g. genetic defects, environmental conditions, maternal diabetes, toxins, like plastic particles, such as microplastics. The inventive heart tissue model may comprise an injury or a healed injury. The inventive method may comprise causing an injury to the tissue model and optionally further a recovery from such an injury. Agents, like test compounds, can be tested to cause such a condition or to test the compound's effects during an injury as well as for or during recovery and/or healing processes. An example injury related effect to be studied is fibrosis after injury and healing of such a fibrosis, e.g. by treatment with a candidate compound.

The inventive tissue model, particular when comprising LV tissue and/or RV tissue, can be used to study chamber dilation, fibrillation, or to study a communication between RV and LV tissue. When comprising atrial tissue or sinoatrial node tissue, it may be used to study atrial fibrillation. Usually, drug effects or environmental effects on heart function have a compartment-specific effect. Such effects can be studies in the inventive tissues, comprising LV, RV, OFT, atria, AVC, SAN and/or AVN tissue. Particularly in the fused tissue model with different tissues present effects on these tissues may be studied in comparison with effects on the other tissues within said tissue model.

Further provided is a method of treating a heart injury in a patient comprising transplanting cells from a heart tissue model of the invention to the injury. The cell may be preferably a cardiomyocyte, a fibroblast, endothelial cell, smooth muscle cell or pacemaker cells, or combinations or mixtures thereof. Especially preferred are cardiomyocytes. Also preferred are pacemaker cells. Pacemaker cells are preferably obtained from sinoatrial node tissue or the sinoatrial node tissue model. It is possible to provide a tissue model of the invention, isolate cells needed for regeneration of an injury, e.g. cardiomyocytes or precursor cells, and provide these cells to the site of the injury. Letting the cells of the tissue model integrate into the wound usually comprises regenerative processes that are improved by the presence of the cells of the inventive tissue models. Said cells are able to connect with the injured heart of the patient and improve its regeneration. An example heart injury is an infarcted heart.

The present invention further comprises a cell culture medium for performing a method of the invention. The medium may be used for the steps as described above. Any particular compound combination as described above may be provided in a medium. In particular, provided is a cell culture medium comprising
a) a bone morphogenic protein, preferably BMP4, a fibroblast growth factor, preferably FGF2, insulin, a Wnt inhibitor, preferably Wnt-C59 or XAV-939, and retinoic acid, with the retinoic acid having a concentration of less than 100 nM;
b) a TGF-beta inhibitor, preferably SB 431542, and a Wnt inhibitor, preferably Wnt-C59 or XAV-939;
c) a Wnt inhibitor, preferably Wnt-C59 or XAV-939, a bone morphogenic protein, preferably BMP4, a fibroblast growth factor, preferably FGF2, insulin, and retinoic acid, preferably with the retinoic acid having a concentration of 50 nM to 500 nM in the medium;
d) a TGF-beta inhibitor, preferably SB 431542, and a Wnt inhibitor, preferably Wnt-C59 or XAV-939;
e) a Wnt inhibitor, preferably Wnt-C59 or XAV-939, a bone morphogenic protein, preferably BMP4, a fibroblast growth factor, preferably FGF2, insulin, and the medium lacking retinoic acid;
f) a TGF-beta inhibitor, preferably SB 431542, a Wnt inhibitor, preferably Wnt-C59 or XAV-939, and retinoic acid, preferably the retinoic acid is in a concentration of 300 nM to 800 nM;
g) a Wnt inhibitor, preferably Wnt-C59 or XAV-939, a bone morphogenic protein, preferably BMP4, a fibroblast growth factor, preferably FGF2, insulin, and retinoic acid, preferably the retinoic acid is in a concentration of 300 nM to 800 nM;
h) a TGF-beta inhibitor, preferably SB 431542, a Wnt inhibitor, preferably Wnt-C59 or XAV-939, a bone morphogenic protein, preferably BMP4 and retinoic acid, preferably the retinoic acid is in a concentration of 300 nM to 800 nM;
i) activin and CHIR99021; preferably the activin is at a concentration of 1 ng/mL to 8 ng/ml, preferably about 5 ng/ml, and/or the CHIR99021 is at a concentration of 1 μM to 6 μM, preferably about 3 μM; or preferably the activin is at a concentration of 30 ng/mL to 100 ng/ml, preferably about 50 ng/ml, and/or the CHIR99021 is at a concentration of 2 μM to 8 μM, preferably about 4 μM; or preferably the activin is at a concentration of 6 ng/mL to 30 ng/ml, preferably about 10 ng/ml, and/or the CHIR99021 is at a concentration of 0.4 μM to 4 μM, preferably about 2 μM; or preferably the activin is at a concentration of 30 ng/mL to 100 ng/mL, preferably about 50 ng/ml, and/or the CHIR99021 is at a concentration of 0.1 μM to 4 μM, preferably about 1 μM; or
j) a Wnt inhibitor, preferably Wnt-C59, a bone morphogenic protein, preferably BMP4, a fibroblast growth factor, preferably FGF2, insulin, and retinoic acid, preferably the retinoic acid is in a concentration of 300 nM to 800 nM, especially preferred 500 nM.

Any of the media may comprises amino acids required for cell growth and an energy source, such as a carbohydrate, especially preferred glucose. They further may comprise salts and ions needed for cell growth, such as Ca, Fe, Mg, K, Na, Zn, Cl, SO₄, NO₃, PO₄ ions. Further preferred components of the medium are vitamins, such as vitamin B12, biotin, choline, folic acid, inositol, niacinamide, pantothenic acid, pyridoxine, riboflavin, thiamine.

Preferred amino acids include essential amino acids, prefer-ably also any one of the following amino acids alanine, argi-nine, asparagine, aspartic acid, cysteine, cystine, glutamic acid, glutamine, glycine, histidine, isoleucine, leucine, lysine, methionine, phenylalanine, proline, serine, threonine, tryptophan, tyrosine, valine.

Any of these media may be combined in a kit of different media or a kit of one or more medium and another means used in the inventive method, e.g. a carrier or mold used for fusion of cultured tissues. The media may be provided in a kit that further comprises instructions for performing the inventive method. Such instructions may be in printed form or in a computer-readable format on a suitable data carrier. A carrier or mold may be a plate with a recess in the shape for fusion of the different tissues as described above. An example is an elongated recess to place a linear arrangement of different heart tissues in contact with each other.

The following numbered embodiments are preferred according to the invention. Any of the numbered embodiments can of course be combined with embodiments and preferred options as described above, or with the corresponding elements of the example sec-tion.
1. A heart tissue model comprising a heart tissue with at least one inner cavity or a central chamber, wherein the heart tissue model comprises at least two different heart tissues selected from left ventricle tissue, right ventricle tissue, atrial tissue, outflow tract tissue, atrioventricular canal tissue, sinoatrial node tissue, and atrioventricular node tissue; wherein the central chamber can be shared by at least two different heart tissues, and/or wherein the at least two different heart tissues comprise a electrophysiological signalling connection or a calcium signaling connection or an ability to propagate a tissue contraction.
2. The heart tissue model of 1, wherein
the left ventricle tissue comprises at least 60% cardiac cells selected from cardiomyocytes, endocardial cells and epicardial cells;
the right ventricle tissue comprises at least 60% cardiomyocytes;
the atrial tissue comprises at least 60% cardiomyocytes;
the outflow tract tissue comprises at least 60% cardiomyocytes;
the atrioventricular canal tissue comprises at least 60% cardiomyocytes;
the sinoatrial node tissue comprises at least 60% cardiomyocytes; and/or
atrioventricular node tissue comprises at least 60% cardiomyocytes.
3. The heart tissue model of 1 or 2, wherein the inner cavity or central chamber is completely surrounded by tissue selected from left ventricle tissue, right ventricle tissue, atrial tissue, outflow tract tissue, atrioventricular canal tissue, sinoatrial node tissue, or atrioventricular node tissue; and/or wherein the volume of the inner cavity or central chamber is not leading into a major blood vessel.
4. The heart tissue model of any one of 1 to 3 having a size in its largest dimension of 0.3 mm to 50 mm.
5. The heart tissue model of any one of 1 to 4, wherein left ventricle tissue cells express one or more expression markers selected from NPPA, IRX4 and HEY2; and/or left ventricle tissue cells lack expression of one or more expression markers selected from NR2F2, TBX2 and TBX3
right ventricle tissue cells express one or more expression markers selected from NPPA, IRX1, IRX2 and PRDX1; and/or right ventricle tissue cells lack expression of one or more expression markers selected from NR2F2, TBX2, and WNT5A;
atrial tissue cells express one or more expression markers selected from NPPA, NR2F1, NR2F2 and HEY1; and/or atrial tissue cells lack expression of one or more expression markers selected from IRX1, IRX4 and HEY2;
outflow tract tissue cells express one or more expression markers selected from WNT5A, MSX1, BMP4, WNT11 and RSPO3; and/or outflow tract tissue cells lack expression of one or more ex-pression markers selected from TBX3, NR2F1 and NPPA;
atrioventricular canal tissue cells express one or more expression markers selected from TBX2, MSX2 and RSPO3; and/or atrioventricular canal tissue cells lack expression of one or more expression markers selected from IRX1, IRX4 and NPPA; sinoatrial node tissue cells express one or more expression markers selected from SHOX2, TBX3, HCN4, ISL1 and GJC1; and/or sinoatrial node tissue cells lack expression of one or more ex-pression markers selected from NKX2.5, IRX1, IRX4 and NPPA; and/or
atrioventricular node tissue cells express one or more expression markers selected from TBX3, TBX5, KCNE1, HCN4 and GJC1; and/or atrioventricular node tissue cells lack expression of one or more expression markers selected from RSPO3, MSX2, IRX4 and NPPA.
6. The heart tissue model of any one of 1 to 5, wherein the size of the inner cavity and/or central chamber at its largest dimension is at least 30% of the size of the heart tissue model at its largest dimension.
7. The heart tissue model of any one of 1 to 6 wherein cardiomyocytes or endocardial cells directly face the inner cavity and/or central chamber.
8. A method to generate a heart tissue model of any one of 1 to 7 comprising generating at least two different heart tissues in vitro, wherein the different heart tissues are selected from left ventricle progenitor first heart field tissue, right ventricle/outflow tract progenitor second heart field tissue, atrial progenitor second heart field tissue, outflow tract progenitor second heart field tissue, atrioventricular canal progenitor second heart field tissue, sinoatrial node tissue, and atrioventricular node tissue, and fusing the at least two heart tissues,
culturing the fused tissue model and letting a calcium signaling connection, ability to propagate a contraction and/or a central chamber between the different heart tissues form.
9. The method of 8, wherein fusing the at least two heart tissues comprises placing the different heart tissues in contact to each other and letting the tissues fuse, preferably by cell growth.
10. The method of 8 or 9, wherein the different heart tissues have been cultured and differentiated from a pluripotent cell and wherein the fusion is at culture day 1 to 7 from a pluripotent stage; preferably right ventricle/outflow tract progenitor second heart field tissue, atrial progenitor second heart field tissue, atrioventricular progenitor second heart field canal tissue, sinoatrial node progenitor second heart field tissue, and/or atrioventricular node tissue is fused at culture day 2 to 5; or preferably left ventricle progenitor first heart field tissue is fused when expressing the expression marker IRX4, TBX5 and/or HEY2; preferably right ventricle progenitor second heart field tissue is fused when expressing the expression marker TBX1, FOXC1 and/or FOXC2; preferably atrial progenitor second heart field tissue is fused when expressing the expression marker HOXB1, TBX5 and/or OSR1; preferably atrioventricular canal progenitor second heart field tissue is fused when expressing the expression marker TBX3, FOXF1 and/or HOXB1; preferably sinoatrial node progenitor second heart field tissue is fused when expressing the expression marker SHOX2, TBX3, HCN4, ISL1 and/or GJC1; and/or preferably atrioventricular node tissue is fused when expressing the expression marker TBX3, TBX5, KCNE1, HCN4 and/or GJC1.
11. The method of any one of 8 to 10, wherein one of the at least two different heart tissues is left ventricle progenitor first heart field tissue and generating left ventricle progenitor first heart field tissue comprises differentiating mesoderm cells into left ventricular precursor cells in a medium comprising a bone morphogenic protein, preferably BMP4, a fibroblast growth factor, preferably FGF2, insulin, a Wnt inhibitor, preferably Wnt-C59 or IWP2, and retinoic acid, with the retinoic acid having a concentration of 5 nM to 100 nM, in the medium.
12. The method of any one of 8 to 11, wherein one of the at least two different heart tissues is right ventricle/outflow tract progenitor second heart field tissue and generating right ventricle/outflow tract progenitor second heart field tissue comprises differentiating mesoderm cells into right ventricular precursor cells in a medium comprising a TGF-beta inhibitor, preferably SB 431542, and a Wnt inhibitor, preferably Wnt-C59 or XAV-939; optionally followed by cultivation in a medium comprising a Wnt inhibitor, preferably Wnt-C59 or XAV-939, a bone morphogenic protein, preferably BMP4, a fibroblast growth factor, preferably FGF2, insulin, and retinoic acid, preferably with the retinoic acid having a concentration of 50 nM to 500 nM in the medium.
13. The method of any one of 8 to 12, wherein one of the at least two different heart tissues is outflow tract progenitor second heart field tissue and generating outflow tract progenitor second heart field tissue comprises differentiating mesoderm cells into outflow tract tissue precursor cells in a medium comprising a TGF-beta inhibitor, preferably SB 431542, and a Wnt inhibitor, preferably Wnt-C59 or XAV-939; optionally followed by cultivation in a medium comprising a Wnt inhibitor, preferably Wnt-C59 or XAV-939, a bone morphogenic protein, preferably BMP4, a fibroblast growth factor, preferably FGF2, insulin, and lacking retinoic acid.
14. The method of any one of 8 to 13, wherein one of the at least two different heart tissues is atrial progenitor second heart field tissue and generating atrial progenitor second heart field tissue comprises differentiating mesoderm cells into atrial tissue precursor cells in a medium comprising a TGF-beta inhibitor, preferably SB 431542, a Wnt inhibitor, preferably Wnt-C59 or XAV-939, and retinoic acid, preferably the retinoic acid is in a concentration of 300 nM to 800 nM; optionally followed by cultivation in a medium comprising a Wnt inhibitor, preferably Wnt-C59 or XAV-939, a bone morphogenic protein, preferably BMP4, a fibroblast growth factor, preferably FGF2, insulin, and retinoic acid, preferably the retinoic acid is in a concentration of 300 nM to 800 nM.
15. The method of any one of 8 to 14, wherein one of the at least two different heart tissues is atrioventricular canal progenitor second heart field tissue and generating atrioventricular canal progenitor second heart field tissue comprises differentiating mesoderm cells into atrioventricular canal tissue precursor cells in a medium comprising a TGF-beta inhibitor, preferably SB 431542, a Wnt inhibitor, preferably Wnt-C59 or XAV-939, a bone morphogenic protein, preferably BMP4 and retinoic acid, preferably the retinoic acid is in a concentration of 300 nM to 800 nM; optionally followed by cultivation in a medium comprising a Wnt inhibitor, preferably Wnt-C59 or XAV-939, a bone morphogenic protein, preferably BMP4, a fibroblast growth factor, preferably FGF2, insulin, and retinoic acid, preferably the retinoic acid is in a concentration of 300 nM to 800 nM.
16. The method of any one of 8 to 15, wherein one of the at least two different heart tissues is sinoatrial node progenitor second heart field tissue and generating sinoatrial node progenitor second heart field tissue comprises differentiating meso-derm cells into sinoatrial node tissue precursor cells in a medium comprising a TGF-beta inhibitor, preferably SB 431542, a bone morphogenic protein, preferably BMP4 and retinoic acid, preferably the retinoic acid is in a concentration of 300 nM to 800 nM.
17. The method of any one of 8 to 16, wherein one of the at least two different heart tissues is atrioventricular node tissue and generating atrioventricular node tissue comprises differentiating mesoderm cells into atrioventricular canal tissue precursor cells in a medium comprising a TGF-beta inhibitor, preferably SB 431542, a Wnt inhibitor, preferably Wnt-C59 or XAV-939, a bone morphogenic protein, preferably BMP4 and retinoic acid, preferably the retinoic acid is in a concentration of 300 nM to 800 nM; and wherein atrioventricular canal progenitor second heart field tissue is further differentiated into atrioventricular node tissue by further maturing in a medium containing an activator and/or inhibitor of sonic hedgehog signaling and/or a BMP.
18. The method of any one of 8 to 17, wherein fusing the at least two heart tissues comprises culturing in a medium comprising a Wnt inhibitor, preferably Wnt-C59, a bone morphogenic protein, preferably BMP4, a fibroblast growth factor, preferably FGF2, insulin, and retinoic acid, preferably the retinoic acid is in a concentration of 300 nM to 800 nM, especially preferred 500 nM.
19. The method of any one of 8 to 18, wherein generating at least two different heart tissues in vitro comprises generating mesoderm cells by differentiating pluripotent cells in a medium comprising activin and a Wnt activator, preferably CHIR99021; preferably at least until differentiated cells express expression markers selected from BRA, EOM, MIXL1, FOXA2, preferably in the absence of SOX2; and/or preferably wherein for mesoderm cells that are further differentiated into left ventricle tissue, the activin is at a concentration of 1 ng/ml to 8 ng/ml, preferably about 5 ng/ml, and/or the CHIR99021 is at a concentration of 1 μM to 6 μM, preferably about 3 μM; for mesoderm cells that are further differentiated into right ventricle tissue, atrial tissue or outflow tract tissue the activin is at a concentration of 30 ng/mL to 100 ng/mL, preferably about 50 ng/ml, and/or the CHIR99021 is at a concentration of 2 μM to 8 μM, preferably about 4 μM; and/or
for mesoderm cells that are further differentiated into atrioventricular canal tissue the activin is at a concentration of 6 ng/ml to 30 ng/ml, preferably about 10 ng/ml, and/or the CHIR99021 is at a concentration of 0.4 μM to 4 μM, preferably about 2 μM; and/or
for mesoderm cells that are further differentiated into sinoatrial node tissue preferably the activin is at a concentration of 30 ng/mL to 100 ng/ml, preferably about 50 ng/ml, and/or the CHIR99021 is at a concentration of 0.1 μM to 4 μM, preferably about 1 μM.
20. A right ventricle tissue model comprising cells expressing expression markers IRX1, IRX2 and PRDX1.
21. An atrial tissue model comprising cells expressing expression markers NR2F1, NR2F2 and HEY1.
22. An outflow tract tissue model comprising cells expressing expression markers WNT5A, MSX1, BMP4 and RSPO3.
23. An atrioventricular canal tissue model comprising cells ex-pressing expression markers TBX2, MSX2 and RSPO3.
24. A sinoatrial node tissue model comprising cells expressing expression markers SHOX2, TBX3, HCN4, ISL1 and GJC1.
25. An atrioventricular node tissue model comprising cells ex-pressing expression markers TBX3, TBX5, KCNE1, HCN4 and GJC1.
26. The tissue model of any one of 20 to 25 comprising at least 60% cardiac cells selected from cardiomyocytes, endocardial cells and epicardial cells.
27. The tissue model of any one of 20 to 26 comprising having a size in its largest dimension of 0.2 mm to 30 mm.
28. The tissue model of any one of claims 20 to 27, comprising an inner cavity.
29. The method of any one of 8 to 19 for screening or testing a candidate compound on its effects on heart development and/or functionality comprising generating a heart tissue model according to any one of 8 to 19 while treating the cells with the candidate compound and comparing development of the heart tissue model with development and/or or functionality of a heart tissue model that was not treated with the candidate compound.
30. A method of observing the effects of mutated, suppressed or overexpressed genes during heart development comprising generating a heart tissue model according to any one of 8 to 19 wherein the cells have a suppressed or mutated candidate gene or overexpress a candidate gene and comparing development of the heart tissue model with development of a heart tissue model that was not generated with a suppressed, mutated or overexpressed gene.
31. The method of screening or testing a candidate compound on its effects on heart development and/or functionality according to 29 in combination with observing the effects of suppressed, mutated or overexpressed genes during on heart development according to 30.
32. A method of screening or testing a candidate compound on its effects on heart functionality comprising treating a heart tissue model according to any one of 1 to 7 or 20 to 28 with the candidate compound and comparing with a functionality of a heart tissue model that was not treated with the candidate compound.
33. A method of treating a heart injury in a patient comprising transplanting one or more cells, preferably cardiomyocytes, from a heart tissue model of any one of 1 to 7 or 20 to 28 to the injury.
34. A cell culture medium comprising
a) a bone morphogenic protein, preferably BMP4, a fibroblast growth factor, preferably FGF2, insulin, a Wnt inhibitor, preferably Wnt-C59 or XAV-939, and retinoic acid, with the retinoic acid having a concentration of less than 100 nM;
b) a TGF-beta inhibitor, preferably SB 431542, and a Wnt inhibitor, preferably Wnt-C59 or XAV-939;
c) a Wnt inhibitor, preferably Wnt-C59 or XAV-939, a bone morphogenic protein, preferably BMP4, a fibroblast growth factor, preferably FGF2, insulin, and retinoic acid, preferably with the retinoic acid having a concentration of 50 nM to 500 nM in the medium;
d) a TGF-beta inhibitor, preferably SB 431542, and a Wnt inhibitor, preferably Wnt-C59 or XAV-939;
e) a Wnt inhibitor, preferably Wnt-C59 or XAV-939, a bone morphogenic protein, preferably BMP4, a fibroblast growth factor, preferably FGF2, insulin, and the medium lacking retinoic acid;
f) a TGF-beta inhibitor, preferably SB 431542, a Wnt inhibitor, preferably Wnt-C59 or XAV-939, and retinoic acid, preferably the retinoic acid is in a concentration of 300 nM to 800 nM;
g) a Wnt inhibitor, preferably Wnt-C59 or XAV-939, a bone morphogenic protein, preferably BMP4, a fibroblast growth factor, preferably FGF2, insulin, and retinoic acid, preferably the retinoic acid is in a concentration of 300 nM to 800 nM;
h) a TGF-beta inhibitor, preferably SB 431542, a Wnt inhibitor, preferably Wnt-C59 or XAV-939, a bone morphogenic protein, preferably BMP4 and retinoic acid, preferably the retinoic acid is in a concentration of 300 nM to 800 nM;
i) activin and CHIR99021; preferably the activin is at a concentration of 1 ng/mL to 8 ng/mL, preferably about 5 ng/ml, and/or the CHIR99021 is at a concentration of 1 μM to 6 μM, preferably about 3 μM; or preferably the activin is at a concentration of 30 ng/mL to 100 ng/mL, preferably about 50 ng/ml, and/or the CHIR99021 is at a concentration of 2 μM to 8 μM, preferably about 4 μM; and/or preferably the activin is at a concentration of 6 ng/mL to 30 ng/ml, preferably about 10 ng/ml, and/or the CHIR99021 is at a concentration of 0.4 μM to 4 μM, preferably about 2 μM; and/or preferably the activin is at a concentration of 30 ng/mL to 100 ng/ml, preferably about 50 ng/ml, and/or the CHIR99021 is at a concentration of 0.1 μM to 4 μM, preferably about 1 μM;
j) a Wnt inhibitor, preferably Wnt-C59, a bone morphogenic protein, preferably BMP4, a fibroblast growth factor, preferably FGF2, insulin, and retinoic acid, preferably the retinoic acid is in a concentration of 300 nM to 800 nM, especially preferred 500 nM.

Throughout the present disclosure, the articles “a”, “an”, and “the” are used herein to refer to one or to more than one (i.e., to at least one) of the grammatical object of the article.

As used herein, words of approximation such as, without limitation, “about”, “substantial” or “substantially” refer to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by e.g. +10%.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The “comprising” ex-pressions when used on an element in combination with a numerical range of a certain value of that element means that the element is limited to that range while “comprising” still relates to the optional presence of other elements. E.g. the element with a range may be subject to an implicit proviso excluding the presence of that element in an amount outside of that range. As used herein, the phrase “consisting essentially of” requires the specified integer(s) or steps as well as those that do not materially affect the character or function of the claimed invention. As used herein, the closed term “consisting” is used to indicate the presence of the recited elements only.

The present invention is further illustrated by the following figures and examples, without being limited to these embodiments of the invention.

### FIGURES

FIG. 1: aSHF and pSHF progenitors express typical markers and form functional cardioids.

A. Differentiation protocol into three main cardiac lineages: first heart field (FHF), anterior second heart field (aSHF) and posterior second heart field (pSHF).

B. Real-time qPCR of TBX1 and TBX5 levels of FHF and aSHF progenitors at d3.5 in 2D, 3D and 2D->3D protocols. Fold change normalized to housekeeping gene and pluripotency.

C. RNAscope staining of TBX1 and TBX5 of aSHF cardioid cryosections at d3.5 showing increased TBX1 expression and absence of TBX5 expression in 2D->3D approach compared to 3D differentiation.

D. Volcano plot of differentially expressed genes at d3.5 of FHF versus aSHF progenitors (top) and aSHF versus pSHF progenitors (bottom).

E. Heatmap reveals upregulation of aSHF and pSHF genes in corresponding differentiation protocol at d3.5. General cardiac genes are more highly expressed in FHF compared to aSHF and pSHF.

F. RNAscope staining (TBX1, TBX5 and HOXB1) of FHF, aSHF and FHF progenitors at d3.5.

G. Immunostaining of aSHF marker (FOXC2) and pSHF marker (FOXF1) (G′) of organoids at d3.5 and quantification of staining (G″) (N=3, n=3-4). Mean±SD.

H. Venn Diagram showing the extent of intersection between aSHF, pSHF and FHF of upregulated genes compared to pluripotency.

I. Three biological replicates at d9.5 show highly robust and efficient differentiation into FHF, aSHF and pSHF cardioids.

J. Cryosection of FHF, aSHF and pSHF cardioids at d9.5 showing expression of CM specific marker MYL7.

K. Quantification of TNNI1-GFP+ cells in cardioids at day 9.5 via flow cytometry. Mean+/−SD. (N=3, n=8)

L. Representative flow cytometry plot of FHF, aSHF and pSHF de-rived-CM using reporter and WT line.

All scale bars in this figure have a length of 200 mm. Used cell lines in this figure: H9 and WTC. *p<0.05, **p<0.01, ***p<0.001, ****p<0.0001.

FIG. 2: a/pSHF-derived cardioids differ in morphology and gene expression

A. Time course of cardioids of the three main lineages from d2.5 until d9.5 reveal delayed cavity formation and delayed expression of TNNI-GFP in SHF compared to FHF lineage.

B. Quantification of cardioid area change during differentiation in all three protocols. (N=3, n=32)

C. & D. Cryosections of cardioids from d2 until d5.5 showing delayed cavity initiation (white arrow) and cavity formation (yellow arrow) in SHF lineages. Immunostaining (C) and mRNA expression quantification (D) of proliferation marker Ki67 over time shows higher proliferation of aSHF at d4.5.

E. Principle component analysis (PCA) Plot of vst using the top 1000 variable genes. VST: variance-stabilized transformed counts.

F. Expression of lineage specific genes over time. aSHF cardioids express RV specific genes, whereas pSHF cardioids express atrial specific genes.

G. Volcano plot shows the differentially expressed genes at d9.5 of FHF versus aSHF (top) and FHF versus pSHF (bottom) cardioids. H. Lineage specific staining of IRX1 (RV marker) and NR2F2 (atrial marker) at d14 and quantification of staining (H′). Each data point represents one organoid. (N=3, n=3-4) All scale bars in this figure have a length of 200 mm.

FIG. 3: SHF protocol optimizations into OFT and AVC cardioids.

A. Differentiation protocol of aSHF progenitors into RV and OFT cardioids by addition or absence of RA during patterning stage 2.

B. RNAseq time course of developing RV and OFT cardioids reveal expression of lineage specific genes.

C. Global gene expression difference of RV and OFT cardioids at d9.5.

D. & E. OFT cardioids highly express the OFT markers ISL1 (D) and WNT5A (E) at d14 compared to RV cardioids.

F. Whole mount images of RV, OFT, Atria and AVC cardioids of three biological replicates at d9.5.

G. Differentiation protocol for Atria and AVC cardioids using different induction conditions and addition of BMP during patterning stage 2 for AVC.

H. Differentially expressed genes at d1.5 between AVC, FHF and SHF progenitors.

I. Volcano plot showing most differentially expressed genes of AVC compared to Atria progenitors at d3.5

J. Expression of AVC organoids over time.

K. AVC cardioids upregulated TBX2 compared to Atria cardioids, but still express TBX3.

L. Addition of R^{A} and FGF, and inhibition of NOTCH and BMP in Atria cardioids from d7.5 onwards leads to upregulation of chamber marker.

M. RV and LV cardioids kept in published maturation media after d7.5 showed increase of chamber specific marker. Atrial cardioids kept in RA, FGF, NOTCHi and BMPi followed by published maturation media downregulate AVC specific marker compared to cardioids in CDMI.

All scale bars in this figure have a length of 200 mm.

FIG. 4: Functional characterization of cardioid subtypes

A. Bar graphs showing what percentage of organoids contract within 1 minute of recording on both day6 and day9.

B. Quantification of how many times different types of organoids beat per min (BPM) at both d6 and d9.

C. Quantification of how many pixels move during the contraction divided by the area of the organoid (extent of contraction). This is a proxy of how far the cardioid's edge moves during one contraction. Organoids which are not beating are not included.

A-C: Were all performed in N=2-7 and for each biological replicate there are 16 technical replicates resulting in 80, 65, 48, 48, and 33 organoids respectively for LV, RV, OFT, Atria, and AVC.

D. RNA-expression of HCN4 throughout the differentiation quantified by bulk RNA-seq. Each dot represents the mean (N=3) and error bars represent the standard deviation. Lines connect dots for ease of following trends

E. Bulk RNA-seq showing calcium channels of both L and T-types on D9.5 of differentiation.

F. Representative calcium signal propagation throughout LV, RV, and Atria cardioids for one beat. The map is colored by the time each pixel reached 50% of peak intensity.

G. Scaled heat map of RNA-seq at D9.5 showing the expression of key ion channels involved in contraction

H. Patch clamp analysis performed on cells dissociated from different RV and Atria cardioids. Shown are representative AP curves for each cell type. Top: RV Bottom: Atria.

I. Action potential duration (APD90) measured from peak to 90% repolarization.

J. Amplitude of AP measured from peak.

K. Resting membrane potential of each cell type.

K-M: Each point represents the mean from one cell. Bars represents mean of cells and error bars represent standard deviation. *p<0.05, **p<0.01, ***p<0.001, ****p<0.0001.

FIG. 5: Fusion of developing cardioids to study developmental electrochemical signal propagation

A. Dissociation of 2D cardiac progenitors at d3.5 and mixing to form cardioids of the same or different progenitors.

B. Cardiac progenitors of different types labelled with H₂B-GPF or LMNB1-RFP are sorted at d7.5, whereas progenitors of the same type are mixed.

C. Cardioids keep their identity upon mixing. RV progenitors still differentiation into the RV CM as shown by IRX1 staining.

D. CDH1 staining of the cardioids at d4.5, 24 hours after mixing of different or the same progenitors.

E. Cardioids can be fused together at D3.5 in different combinations by fusing either two or three different compartments together. By doing this with a fluorescent green and red lines we can track exactly how different compartments interact with each other.

F. Representative bright field image from fused cardioid at d6.5 of an atrial (A), LV and RV cardioids.

G. Representative calcium signal propagation through a 3 chambered-cardioid for one beat. The map is colored by the time each pixel reached 50% of peak intensity.

H. Cardioids keep identity upon fusion and all compartments homogenously express the CM marker TNNT2.

I. Percent of organoids beating for each fusion type, for d6 and d9.

J. Shows the beats per minute (BPM) for the different fusion types on both d6 and d9.

K. Shows in which cardioid the beat is initiated in (indicated by color) as a percentage of all cardioids recorded. Mix indicates that the beat was initiated by different organoids for each beat and None indicates organoids where fused cardioids are not beating at all or if there is no interaction between the fused cardioids. Shown for both day 6 and day 9.

L. The timing of cardiac progenitor fusion was then optimized to promote the formation of a shared cavity between cardioids by aggregating FHF progenitors at d1.5 and aggregating the a/pSHF progenitors at d3.5 and fusing the aggregates 4 hours after aggregation.

M. Representative image of a cardioid with three compartments using the protocol depicted in M. Atria is marked in red, LV in gray and RV in green.

N. Representative cryosection of the triple fused cardioids using the protocol depicted in M stained with TNNT2, a pan cardiac marker. Arrow indicates shared cavity between chambers.

O. Cryosection of fused cardioids of two different compartments using the protocol depicted in M. Fused cardioids share some cavities indicated by blue arrow and express TNNT2.

All scale bars in this figure have a length of 200 mm. *p<0.05, **p<0.01, ***p<0.001, ****p<0.0001.

FIG. 6: Validation of cardioid subtypes by different KO phenotypes

A. ISL1 KO cardioids show drastic decrease in size using atrial and OFT protocol and a slight decrease in size using LV and RV protocol at d9.5.

B. Cross-section of cardioids show decreased TNNT2 expression in RV, Atria and OFT protocols in ISL1 KO compared to WT.

C. RNAseq analysis showing mis-regulated genes in ISL1 KO cardioids compared to WT at d9.5.

D. OFT ISL1 KO cardioids show a switch in identity and express the atrial marker NR2F2. Atria and OFT cardioids express less TNNT2 in the ISL1 KO line compared to WT.

E. Contraction analysis at d9 reveals that Atria WT cardioids have more beats per minute (BPM) compared to Atria ISL1 KO. At d14 Atria ISL1 KO cardioids have a higher contraction rate compared to WT. At d14 OFT ISL1 KO cardioids start to contract, while OFT WT cardioids do not contract. (N=1, n=24)

F. Global gene expression differences of OFT cardioids using ISL1 KO line compared to WT line at d9.5.

G. Atria and AVC progenitors do not express HOXB1, a pSHF marker, in TBX5 KO compared to WT.

H. Representative whole mount images of TBX5 KO and WT cardioids and quantification (H′) of cardioid area at d9.5 (N=3, n=8).

I. TBX5 KO cardioids downregulate NPPA and TNNT2 in LV and RV protocol compared to WT. Atria and AVC TBX5 KO cardioids fail to differentiate into CMs.

J. RNAseq analysis showing differentially expressed chamber specific genes (NPPA, NPPB) and identity genes of TBX5 KO compared to WT cardioids at d9.5.

K. Global gene analysis of cardiac progenitors at d3.5 shows mis-regulated genes of FOXF1 KO compared to WT.

L. Time course of Atrial and AVC cardioids in FOXF1 KO and WT.

M. Representative Real-time qPCR at d3.5 comparing Atria and AVC progenitors using FOXF1 KO and WT line. Fold change normalized to housekeeping gene and pluripotency.

N. Decreased cardioids size of FOXF1 KO cardioids using the LV and AVC protocol compared to WT cardioids at d9.5. RV and Atrial cardioids have the same size in FOXF1 KO and WT line at d9.5.

O. Contraction analysis at d6.5 and d9.5 of LV, Atrial and AVC cardioids showing reduced BMP at d6.5 in FOXF1 KO compared to WT. (N=1, n=16)

All scale bars in this figure have a length of 200 mm. *p<0.05, **p<0.01, ***p<0.001, ****p<0.0001.

FIG. 7: Multichambered cardioid platform for screening terato-gen-induced cardiac defects

A. Whole mount images of cardioids treated with different concentration of Thalidomide starting from mesoderm induction compared to control cardioids (d9.5). A′. Quantification of cardioid area at d9.5 of cardioids treated with thalidomide. (N=1, n=8)

B. Representative real-time qPCR from d9.5 cardioids treated with thalidomide showing mis-regulation of lineage specific genes. Fold change normalized to housekeeping gene and pluripotency.

C. Comparison of size and TNNI-GFP expression of d4.5 cardioids treated with different concentrations of acitretin.

D. Inefficient CM differentiation and morphological changes of cardioids treated with Acitretin.

E. Representative real-time qPCR from d3.5 and d9.5 cardioids treated with actitretin. Fold change normalized to housekeeping gene and pluripotency.

F. Whole mount images of cardioids treated with BPAs (B), PFOs (P) and Nanoplastic (NP) compared to control cardioids (d9.5).

All cardioids were treated with the teratogens from mesoderm induction (day 0) until day 9.5. All scale bars in this figure have a length of 200 mm. *p<0.05, **p<0.01, ***p<0.001, ****p<0.0001.

FIG. 8

A. Immunostaining of TBX5 and FOXC2 of aSHF cells at day 3.5 using 3D vs 2D->3D protocol.

B. SOX2 and EOMES staining after mesoderm induction for the aSHF (d1.5) of 3D vs 2D->3D protocol.

C. Heatmap of head mesoderm makers of all progenitor populations at d3.5.

D. Real-time qPCR of TBX1 and TBX5 level testing factors that are important for aSHF development alone and in combination.

E. Optimization of aSHF protocol by testing different BMP and RA concentrations during patterning 2 stage.

F. Optimization of pSHF testing different factors during patterning 2 stage in 2D and 3D (Cyclo: Cyclopamin, CH: CHIR99021, B: BMP4, I: Insulin).

G. RNAscope staining of TBX1 and TBX5 of aSHF progenitors using different Activin concentrations during mesoderm induction.

H. TBX5 staining of all three progenitors at d3.5.

I. Homogenous NKX2-5 expression and absence of SOX2+ cells in all progenitors at d3.5.

J. Low cell density during mesoderm induction leads to homogenous TNNI expression at d9.5 in aSHF derived cardioids. J′. SOX1/2+core of aSHF cardioid when using high cell density during mesoderm induction.

K. Cardioids of all lineages showing very few cells expressing PECAM1 and FOXA2, and absence of COL1A1 and SOX2

L. 2D 24 well plate endothelial cell differentiation of all three progenitor populations.

All scale bars in this figure have a length of 200 mm.

FIG. 9

A. Lineage specific staining of HEY2 (LV marker) of LV, RC and atrial cardioids.

B. RV (IRX1) and atria (NR2F2) specific staining of cardioids derived from hESC line H9.

C. Venn Diagram showing the extent of intersection between LV, RV, Atria at d9.5 of upregulated genes compared to pluripotency.

D. Volcano plot of differentially expressed genes at d9.5 of RV versus Atria.

All scale bars in this figure have a length of 200 mm.

FIG. 10

A. GO-term analysis of RV and OFT cardioids at d4.5.

B. Optimization of RA concentrations during patterning stage 2 revealed that RA500 leads to higher upregulation of RV and chamber specific genes.

C. High protein expression of HAND1 and HAND2 in OFT cardioids compared to RV cardioids.

D. Quantification of cardioid area change during differentiation in all five protocols. (N=3, n=32)

All scale bars in this figure have a length of 200 mm.

FIG. 11

A. Representative curve tracking contraction rate using bright field imaging and MuscleMotion software.

B. Representative images showing the extent of contraction for different organoid types. The red pixels represent which pixels changed during the contraction.

C. Calcium traces showing f/f0 for a time span of 30 seconds with different beat patterns for each cardioid.

D. FluoroVolt-AP curves recorded from 3D cardioids for FHF, RV, Atria, and AVC. Each column represents a different cardioid and overlayed in each quadrant is a different position of the cardioid.

E-G. APD90, APD50, and APD30 from FluoroVolt in 3-D cardioids. Each point represents one cardioid and APDs have been averaged across beats and across different locations of the cardioid. Error bar represents standard deviation across cardioid type N=2 n=5 for LV and Atria and N=1 n=3 for RV and AVC.

H. CAPD90 for patch clamp data shown in FIG. 4K. Corrected using Fridericia correction method

I. Beat Interval of the cells that were recorded in FIG. 4K.

FIG. 12

A. Representative widefield images of progenitor populations 1 day after mixing. Showing a degree of separation between different progenitor populations.

B. Sectioned cardioids of each mixing condition stained for TNNT2.

C. Differences of Cadherin expression in control LV, RV and atrial cardioids over time.

D. Control conditions of LV, RV, and Atrial progenitor cardioids stained for CDH2 and CDH1 on D3.5 of the differentiation.

E. Cardioids being fused together at different times during the differentiation. The cardioids were either put together on d3.5 or D5.5. The images were then taken on D9.5. (R=biological replicate). Cardioids fused on D3.5 are more connected with each other compared to D5.5.

F. Cardioids one day after fusing (day 4.5). On the right side with the RV labelled with LMN1-RFP and FHF with H₂B-GFP, then on the left vice versa.

G. Time lapse of calcium signaling travelling through a 3 chambered cardioid.

H. Time lapse of the contraction of a two chambered cardioid.

I. ICC-staining of 2-chambered Cardioids. Where LV cardioids are colored in grey, RV cardioids in green and Atria cardioids in red. Stained for NR2F2, IRX1, and TNNT2.

All scale bars in this figure have a length of 200 mm.

FIG. 13

A. Validation of ISL1 KO line of all protocols at d3.5.

B. RNAseq analysis showing mis-regulated genes in ISL1 KO vs WT at d3.5 in all protocols.

C. Strong downregulation of WNT5A (OFT marker) in OFT ISL1 KO cardioids compared to WT at d14.5.

D. Extent of contraction analysis reveals less contraction in atrial ISL1 KO cardioids compared to WT at d9.5, but simi-lar contraction at d14.5.

E. Time course of RV, atria and OFT cardioid formation using ISL1 KO and WT line

F. Quantification of cardioid area of ISL1 KO and WT cardioids at d3.5 (N=4, n=8-24) and 9.5 (N=2, n=8-24).

G. RNAseq analysis of TBX5 KO and WT cardioids showing mis-regulated aSHF and pSHF specific genes.

H. Validation of TBX5 KO line of LV, RV atria and AVC cardioids at d3.5.

I. Representative RT-qPCR of Atria and AVC TBX5 KO cardioids compared to WT at d3.5 and d9.5. Fold change normalized to housekeeping gene and pluripotency.

J. Validation of FOXF1 KO line of atria and AVC cardioids at d3.5.

K. Area analysis of WT and FOXF1 KO organoids of all protocols at d9.5. (N=3-4 (LV, RV, Atria, AVC) and N=1 (OFT), n=8-16)

L. TNNT2 expression of FOXF1 WT vs KO cardioids at d9.5.

M. RNAseq analysis of LV RV and atrial cardioids at d9.5.

N. Representative RT-qPCR of Atria and AVC cardioids of FOXF1 KO compared to WT. Fold change normalized to housekeeping gene and pluripotency.

All scale bars in this figure have a length of 200 mm. *p<0.05, **p<0.01, ***p<0.001, ****p<0.0001.

FIG. 14

A. Whole mount images of cardioids treated with Aspirin showing no size and TNNI-GFP expression differences compared to untreated cardioids.

B. Real time qPCR of cardioids treated with Aspirin compared to control cardioids showing no obvious gene expression differences.

C. Representative whole mount images of cardioids treated with different concentrations of acitretin and quantification (H′) of cardioid area at d9.5 (N=1, n=8).

D. OFT TNNI-GFP signal qualification revealed higher GFP signal in OFT cardioids treated with 5 nM and 10 nM acitretin com-pared to untreated cardioids at d4.5 and d9.5. (N=1, n=8)

E. Whole mount images and quantification (C′) of cardioids treated with All trans Retinol at d9.5 revealed no size difference between treated and untreated cardioids within one protocol the except LV cardioids treated with 50 nM com-pared to untreated.

F. Real time qPCR showing downregulation of OFT and upregulation of ventricular genes in OFT cardioids treated with All trans Retinol.

G. Whole mount images showing less efficient CM differentiation of RV and atria cardioids when treated with different combinations of Plastic residuals.

All cardioids were treated with the teratogens from mesoderm induction (day 0) until day 9.5. *p<0.05, **p<0.01, ***p<0.001, ****p<0.0001.

FIG. 15

A. Differentiation protocol into three cardiac lineages derived from the pSHF: Atrial, sinoatrial node (SAN), and Atrioventricular canal. Showing that the atrial cardioids previously described can be pushed into a more mature state, a novel differentiation of the SAN by changing both the induction and patterning stages and showing how the atrioventricular canal cardioids may be able to be pushed into the atrioventricular node identity.

B. Validation of the sinoatrial node protocol showing protein expression of SHOX2 (a key SAN marker), NKX2.5 (which is absent in some parts of the SAN), and HCN4 (a key SAN marker)

C. Heat map showing bulk-RNA expression at day3.5 of SAN differentiation and that it has similar gene profiles to the atria at this time point.

D. Heat map showing bulk-RNA expression at day 9.5 of SAN differentiation and that it upregulates SAN markers compared to the atria protocol.

E. Analysis of contraction rate showing that the SAN beats faster than the other cardioids and reaches more towards the rate of the paced heart.

FIG. 16

A. Immunostaining of NKX2-5 and SOX2 on cross-sections of FHF, aSHF, and pSHF cardioids at day 3.5 (see FIG. 8I).

B. and B′ Aggregation density (at day 3.5) optimization of FHF, aSHF and pSHF progenitors results in different cardioid formation efficiencies later.

C. Beating frequency of LV, RV, and atrial cardioids in the 178/5 hPSCs line.

D. OFT progenitors form alpha-SMA-, SM22-, and Calponin-positive smooth muscle cell progenitors.

FIG. 17

A. Cell-counting and cell size time-course for different cardioid subtypes. (E) Expression of lineage-specific genes over time shown by bulk RNA-seq.

B. Specific NR2F2 immunostaining in atrial cardioids.

C. LV, RV and atrial cardioids form from the 178/5 hiPSC cell line.

D. Outline of cardioid maturation conditions.

E. MYL2, NPPA and NPPB chamber marker expression upon RV and LV cardioid maturation.

F. Marker expression upon atrial cardioid specification and maturation.

G. Immunostaining for the MYL2 cardiac maturation marker in matured LV and RV cardioids.

FIG. 18

A. Whole-cardioid TNNI expression in matured RV and LV cardioids.

B. The ratio of MYH7/MYH6 increases in matured cardioids.

C. Fishbone sarcomere structure in matured cardioids, visualized by alpha-Actinin immunostaining.

D. Contraction analysis of matured cardioids.

FIG. 19

A. sCRNAseq of multi-chamber cardioids confirms compartment-specific identities. 10× Genomics scRNAseq of 5 cardioid subtypes with clustering and showing key markers for every compartment.

B. scRNAseq reveals compartment-specific identities defined by marker combinations. Selection of key compartment markers to determine the marker combinations specific for each compartment.

C. Imposition of key compartment markers to determine on the clustering, showing the overlap of the different identities with the clusters.

### EXAMPLES

### Example 1: Cardioid Generation

### Cell Lines

The WiCell Institute (USA) provided human H9 (female) ES cell lines. The WTC iPS cell line (male, skin fibroblast-derived) was purchased from the Coriell Institute for Medical Research (USA). The Allen Institute for Cell Science's reporter cell line is de-rived from the WTC cell line and received from the Coriell Institute for Medical Research (USA).

The E8 culture system (Chen et al. (2011) Nat. Methods 8, 424-429) was used to cultivate all human pluripotent stem cell lines in a customized in-house medium. 0.5 percent BSA (Europa Biosciences, #EQBAH70), in-house manufactured FGF2, and 1.8 ng/ml TGFb1 were added to the original E8 mix (R&D RD-240-B-010). Cells were cultured on Vitronectin XF (Stem Cell Technologies, #7180) coated Eppendorf (Eppendorf SE, #0030 721.110) or TPP (TPP Techno Plastic Products AG, #92012) tissue culture-treated plates and passaged every 2-4 days at approximately 70 percent confluency using Try-pLE Express Enzyme (GIBCO, #12605010). The absence of Mycoplasma contamination in cells was regularly tested.

### Generation of ISL1, TBX5, and FOXF1 Knock-Out Cell Lines

ISL1, TBX5, and FOXF1 were knocked out in WTC cells using CRISPR/Cas9 multi-guide sgRNAs (Synthego) for target sites on Exon 3 for ISL1, Exon 5 for TBX5, and Exon 1 for FOXF1 (Figures M1I-K). Cells were transfected using the P3 Primary Cell 4D-Nucleofector X Kit S (Lonza-BioResearch, #: V4XP-3032) and Amaxa 4D-Nucleofector (Lonza-BioResearch). Post nucleofection, cells were incubated in E8 supplemented with 5 μM Y-27632 (Tocris, #72302) on a 6-well plate previously coated with Vitronectin XF (StemCell Technologies, #7180). After two days, the medium was changed to E8 without Y-27632 every other day.

Once cells were approx. 70% confluent, single-cell seeding was performed, and the rest of the cells were collected for gDNA extraction. Successful editing was first assessed on a pool level using agarose gels and Sanger sequencing. Subsequently, single colonies were picked and genotyped to confirm a knockout. Colonies were collected with the help of a microscope (EVOS) and transferred into a pre-coated 96-well plate (Corning, Cat #CLS3370) with 150 μl E8/well supplemented with 5 μM Y-27632 and Antibiotic-Antimycotic. Genome editing on a pool and clonal level was analyzed using Synthego's online tool ICE (https://ice.synthego.com/#/).

### Cardioid Generation

hPSCs (WTC or H9 lines) are seeded in a 24-well plate (TPP, #92024) at 30-40 k cells per well in E8 medium+ROCKi (5 μM Y-27632, Tocris #1254). All differentiation media are based on CDM that contains 5 mg/ml bovine serum albumin (Europa Biosciences, #EQBAH70) in 50% IMDM (Gibco, #21980065) plus 50% F12 NUT-MIX (Gibco, #31765068), supplemented with 1% concentrated Lipids (Gibco, #11905031), 0.004% monothioglycerol (Sigma, #M6145-100ML) and 15 μg/ml of transferrin (Roche, #10652202001) (Mendjan et al., Cell Stem Cell 2014, 15:310-325; and Hofbauer et al., Cell 2021, 184 (12): 3299-3317.e22). The medium contains BSA which is important for efficient cardioid generation. 24 hours after seeding in the 24-well plate, the cells are induced with mesoderm induction media. Mesoderm induction media is made up of CDM containing FGF2 (30 ng/ml, QKine, Cambridge/UK), LY294002 (5 μM, Tocris, #1130), Activin A (specific concentrations for different cardioid subtypes-see below, QKine, Cam-bridge/UK), BMP4 (10 ng/ml, R&D Systems RD-314-BP-050), and CHIR99021 (specific concentrations for different cardioid subtypes—see below, R&D Systems RD-4423/50). After 36-40 hours cells are dissociated with TrypleE (Gibco, #12605010) and seeded in a Corning ultra-low attachment 96 well plate (Corning, #7007) at 15-20 k cells/well in Cardiac Mesoderm Patterning Media One made up of CDM containing ROCK inhibitor and for all protocols besides the LV 1 μg/ml of insulin (Roche, #11376497001) plus specific factors depending on cardioid subtype (see below). After seeding, the cells are spun down in a centrifuge for 4 mins at 200 g. This protocol is termed 2D-3D standard protocol. Alternatively, hPSCs were seeded into Corning ultra-low attachment 96 well plate with a density of 5000 cells/well. Cells were seeded in a volume of 200 ml containing E8+ROCKi and collected by centrifugation for 5 minutes at 200 g. As another option, 2500 cells/well were seeded directly into induction media+ROCKi and collected by centrifugation for 5 minutes at 200 g. For both protocols, cells were induced with mesoderm induction media as described for the 2D->3D protocol. These were termed 3D protocols. For both protocols, 24 hours later (or at day 2.5) the cells are fed with Cardiac Mesoderm Patterning Media One. For the next two days, the medium is changed to Cardiac Mesoderm Patterning Media Two made up of CDM containing specific factors depending on the cardioid subtype (see below) and exchanged every day. For the subsequent two days, media is exchanged every day with Cardiomyocyte Differentiation Media CDM medium containing BMP4 (10 ng/ml), FGF2 (8 ng/ml) and insulin (10 μg/ml). For the subsequent days of culture, media is exchanged every other day with CDM containing insulin (10 μg/ml).

Alternatively, the whole protocol can be done in 2D completely by seeding 80,000-170.000 cells/24 well coated with vitronectin and adding the medium on the same timeline as the cardioids. This was termed 2D differentiation.

### Mesoderm Induction Media (Day 0-1.5)

For left ventricle (LV-FHF progenitor derived) cardioid differentiations Activin is used at 5 ng/ml and CHIR99021 at 3 μM. For right ventricle (RV), atria and outflow tract (OFT) differentiations Activin is used at 50 ng/mL and CHIR99021 at 4 μM. For AVC Activin is used at 10 ng/ML and CHIR99021 at 2 μM. For SAN Activin is used at 50 ng/ml and CHIR99021 at 1 μM. These mesoderm induction conditions result in high (>70%) efficiency, homogeneity, and reproducibility of different cardioid subtypes. This stage is characterized by the expression of the BRA, EOM, MIXL1, FOXA2 and other early mesoderm markers and the absence of SOX2, a pluripotency and early neural marker. This medium works best for WTC lines.

### Alternative Mesoderm Induction Media (Day 0-1.5)

For LV (FHF-derived) cardioid differentiations Activin is used at 5 ng/mL and CHIR99021 at 1 μM. For RV (aSHF-derived) and atria (pSHF-derived) differentiations Activin is used at 50 ng/mL and CHIR99021 at 1.5 μM. This medium works best for H9 lines.

### Cardiac Mesoderm Patterning Media One (Day 1.5-3.5)

For LV (FHF progenitor): BMP4 (10 ng/ml), FGF2 (8 ng/ml), insulin (10 μg/ml), C59 (2 UM, Tocris, #5148/10) and retinoic acid (50 nM, Sigma Aldrich, #R2625). The retinoic acid concentration should be low as a higher concentration (>100 nM) leads to FHF-derived atria, not ventricular fate. The early specific markers of the LV are IRX4 and HEY2, characterized in Hofbauer et al., Cell 2021, 184 (12): 3299-3317.e22.

For RV (aSHF progenitor): The TGF-beta inhibitor SB 431542 (10 μM, Tocris, #1614/10) and either C59 (2 μM) or XAV-939 (5 UM, SelleckChem, #S1180). The use of SB 431542 leads to reaching SHF lineage and therefore RV, OFT, SHF-derived atria and AVC fate. The earliest aSHF markers at day 3.5 are TBX1 and FOXC1/2 and are shared by RV and OFT.

For OFT (aSHF progenitor): SB 431542 (10 μM) and XAV-939 (5 μM). Same as RV at this stage.

For atria (pSHF progenitor): SB 431542 (10 μM), XAV-939 (5 μM) and retinoic acid (500 nM). The combination of SB and high retinoic acid concentration results in pSHF progenitor identity at day 3.5 characterized by high HOXB1, TBX5 and FOXF1, and absence of TBX1 or IRX4 expression.

For AVC and AVN (pSHF/AVC progenitor): SB 431542 (10 μM), XAV-939 (5 μM), retinoic acid (500 nM) and BMP4 (10 ng/ml). The combination of SB, high retinoic acid and additional BMP4 at this stage results in the pSHF/AVC progenitor identity characterized by high MSX2 and TBX2 expression in addition to the standard pSHF markers.

The sinoatrial node (SAN)—similar as for AVC but during the induction step high activin and low WNT activation (activin 50 ng/ml and CHIR99021 1 μM) are used. Optionally, also during patterning 1: no WNT inhibition, otherwise as for AVC.

### Cardiac Mesoderm Patterning Media Two (Day 3.5-5.5)

For LV: BMP4 (10 ng/ml), FGF2 (8 ng/ml), insulin (10 μg/ml), C59 (2 μM) and retinoic acid (50 nM) (see Hofbauer et al., above).

For RV: either C59 (2 μM) or XAV-939 (5M), BMP4 (10 ng/ml), FGF2 (8 ng/ml), insulin (10 μg/ml), and retinoic acid (50-500 nM range). The retinoic acid addition here helps drive the RV identity.

For OFT: XAV-939 (5 μM), BMP4 (10 ng/ml), FGF2 (8 ng/ml) and insulin (10 μg/ml). The absence of retinoic acid at this stage results in a specific OFT identity characterized by high WNT5A, MSX1, BMP4 and RSPO3 expression.

For Atria, AVC, SAN and AVN: XAV-939 (5 μM), BMP4 (10 ng/ml), FGF2 (8 ng/ml), insulin (10 μg/ml), and retinoic acid (500 nM). For SAN, same but no WNT inhibition (no XAV-939) or even WNT activation (CHIR99021). For AVN, same as for AVC with additional push into AVN direction, e.g. with BMP and sonic hedgehog signaling (FIG. 15). Atria will start expressing NR2F1/2 and HEY1 at this stage and AVC high levels of TBX2, MSX2 and RSPO3.

### Example 2: Cardioids

### Example 2.1: Chamber Specification Protocol

For the atria specification protocol, atrial cardioids at day 7 were transferred in CDM medium containing Retinoic acid (500 nM, Sigma Aldrich, #R2625), FGF2 (15 ng/ml, Cambridge University), LDN-193189 (200 nM, Stemgent, #04-0074) and LY-411575 (3 μM, Med-ChemExpess, #HY-50752) until day 10. From day 10 until day 21, atrial cardioids were transferred in DMEM with low glucose (1 g/L, Sigma Aldrich, #G8644) containing Dexamethasone (250 nM, Sigma Aldrich, #D4902), Indomethacin (50 μM, Sigma Aldrich, #I7378), T3 hormone (4 nM, Sigma Aldrich, #T6397) and chemically defined lipid concentrate (1×, Invitrogen, #11905031).

For the ventricular specification protocol, LV and RV cardioids on day 7 were transferred in DMEM with high glucose (4, 5 g/L, Sigma Aldrich, #G8644) containing IGF2 (25 ng/ml), CHIR99021 (1 μM, R&D Systems RD-4423/50) and human insulin (10 ng/ml, Sigma) until day 14. From day 14 until day 16, LV and RV cardioids were kept in DMEM with high glucose (4.5 g/L) containing XAV-939 (4 μM, SelleckChem, #S1180). Finally, from day 14 until day 16, LV and RV cardioids were transferred in DMEM with low glucose (1 g/L, Sigma Aldrich, #G8644) containing Dexamethasone (250 nM, Sigma Aldrich, #D4902), Indomethacin (50 μM, Sigma Aldrich, #17378), T3 hormone (4 nM, Sigma Aldrich, #T6397) and chemically defined lipid concentrate (1×, Invitrogen, #11905031).

### Example 2.2: 2D Endothelial Cell Differentiation

hPSCs were seeded at 100,000 cells/24 well coated with vitronectin in E8 medium with 5 mM ROCK-i added. The following day, cells were induced with FLyAB and 1-3 mM CHIR99021 for H9 cells and incubated for 36-40 hours. For the next two days, the medium was exchanged to their respective Cardiac Mesoderm patterning media 1 for the FHF, aSHF, and pSHF. After that, CDM with 200 ng/ml VEGF (200 ng/ml, Peprotech, #AF-100-20) and 2 mM Forskolin (Sigma-Aldrich, #F3917) was given for 2 days, and then the cells were cultured for 1 day in CDM with 100 ng/ml VEGF.

### Example 2.3: Mixing of Progenitors

Cardiac differentiation of different progenitor cell populations (FHF, aSHF, and pSHF) was done in 24 well plates coated with vitronectin until day 3.5 (2D differentiation). Cell populations were labeled using different colored cell lines (WTC: H2B-GFP, WTC: LMNB1-RFP). On day d3.5, progenitor cells were dissociated by adding 200 ul Try-pLE Express Enzyme (GIBCO, #12605010) for 3-4 min at room temperature. Dissociation was stopped by adding 1 ml of CDM containing ROCKi (5 mM). After centrifugation for 4 min at 130 g, cells were resuspended in CDM containing ROCKi (5 mM). Then, two progenitor populations were mixed by seeding 15000-20000 cells per progenitor population into ultra-low attachment (corning) into Co-development Patterning Media, containing C59 (2 μM), BMP4 (10 ng/ml), FGF2 (8 ng/ml), insulin (10 μg/ml), and retinoic acid (500 nM), and ROCKi (5 mM). On day 5.5, media was exchanged to Cardiomyocyte Specification Media for the following two days.

### Example 2.4: Preparation for Multi-Chamber Cardioids

Cardioids are generated as above up until day 3.5 of the differentiation. On day 3.5, developing cardioids are combined in two different manners (see description below) depending on whether double or triple-or multi-fusions are desired. The media used for the fusion condition is the CDM-based Cardiac Mesoderm Patterning Media Two for the LV, RV and Atria containing: BMP4 (10 ng/ml), FGF2 (8 ng/ml), insulin (10 mg/ml), C59 (2μ M) and retinoic acid (500 nM). The fused cardioids are fed the subsequent 2 days every day with Cardiomyocyte Differentiation Media. For the remaining time every other day with CDM containing insulin (10 μg/ml).

### Example 2.5: Generation of Multi-Chambered Cardioids

### Double Fusions

Developing organoids are transferred on day 3.5 using wide opening tips from individual wells of the 96-well Corning ultra-low attachment plate to sharing wells with one other desired organoid subtype. This can be accomplished with any combination of LV, RV or atrial cardioids. For this type of fusion, cardioids were put together in the Co-development Patterning Media. Alternatively, on day 1.5 LV differentiation can be combined with an RV or atrial progenitor differentiation of day 3.5 in Co-development Patterning Media or Cardiac Mesoderm Patterning Media One to get a multi-chambered cardioid with at least one shared cavity. Importantly, the two-chamber/multi-chamber cardioids will co-develop and share a cavity if fused at these early stages. Later fusion (e. g. from day 5.5 on) will impair formation of a shared cavity.

### Triple Fusions

Molds (made from silicone) were created with a shape to place the early cardioids that are to be fused in contact with each other in an order as expected in the natural heart (e.g. a linear order). The molds are sterilized by covering them with 70% ethanol for at least one hour with UV of the laminar flow turned on. The molds are then coated with an anti-adherence-rinsing solution (STEMCELL Technologies, #07010) for a couple of seconds and immediately washed once with PBS. After washing, the molds are kept in the fridge until the day of fusion or used immediately. On day 3.5 of cardiac differentiation, the cardioids are transferred to the molds using wide opening tips. By using molds, the cardioids can be arranged in the desired orientation (e. g. first atria, then LV and RV, as in vivo). Media is not changed while cardioids are fusing in the molds from day 3.5-5.5. The early timing improves fusion efficiency and yields better morphology. Fusions with more developed cardioids may not be complete, depending on the development stage. On day 5.5, the fused cardioids are moved back to the 96-well plate and media change continues as described above.

To track which cardioids in the fusions arise from which cell population, colored cell lines (WTC: H2B-GFP, WTC: LMNB1-RFP) or dyes were used. For this, cells were stained for one hour before induction using SP-DiIC18 (3) (Invitrogen, #D7777) to fluoresce at 564 nm or DiIC18 (5) (Invitrogen, #D12730) to fluoresce at 668 nm.

### Example 2.6: Molds for Multi-Chamber Cardioids

Embedding molds have been designed in Tinkercad and were adjusted in diameter and length based on the cardioid size on the day of fusion. Files were exported as .stl files and loaded into the slicer software XYZ print 1.4.0. The negative was printed using transparent PLA with 100% infill density and 0.1 mm layer height, and 215° C. nozzle temperature. After printing, the negative was treated with a Heatgun (Bosch Hot Air Blower 1800W) at 550° C. to carefully melt the surface of the negative, create a smooth finish and remove the 3D printing typical rough surface. The positive was then cast using polydimethylsiloxane (PDMS). In brief, 5 ml of curing agent and 45 ml of Monomer (both Sylgard® 184 Elastomer Kit, VWR) were mixed intensively. The mixture was then spun down to remove air bubbles and directly used. To reduce the extent of bubbles formed during curing, the molds were cast at a low temperature (40° C.). For this, the negative was placed into a 10 cm dish and slowly covered with 30 ml of the liquid PDMS mixture. The negative was then carefully removed from the polymerized PDMS, and residual PDMS was cut off using a scalpel. The mold was then stuck to the bottom of a clean 10 cm dish using about 5 ml of PDMS and cured at 40° C. To sterilize the mold, it was washed in 70% Ethanol for about 30 min in the fume hood with UV turned on. For positioning cardioids in the mold, the mold was rinsed once with PBS and then coated with an anti-adherence rinsing solution (StemCell Technologies, #07010) to increase the non-stick behavior of the PDMS further. After coating, the molds were rinsed once with PBS and were then ready to use.

### Example 2.7: Analysis Methods

### Cryosectioning

Cardioids were fixed with 4% PFA in PBS and cryoprotected with 30% sucrose in PBS before embedding. The embedding was carried out using the O.C.T. cryo embedding medium (Scigen, #4586K1). Embedded tissues were frozen using a metal surface submerged in liquid nitrogen and stored in a −80° C. freezer until sectioning on a Leica cryostat. Sections were collected on SuperFrost Plus slides (Thermo Fisher Scientific, #10149870) and kept at −20° C. or −80° C. until immunostaining.

### Immunostaining

To remove O.C.T., fixed specimens were washed in 1×PBS for 15 min. Optionally tissues were placed in permeabilization solution 0.5% Triton-X100 (Sigma-Aldrich, #T8787) for 5 mins to increase antibody permeabilization. Tissues were then incubated in blocking solution (PBS (GIBCO, #14190094) with 4% donkey serum (Bio-Rad Laboratories, #C06SB) and 0.2% TritonX-100 for at least 30 min. Subsequently, specimens were incubated for 3 hours at room temperature or overnight at 4° C. in a blocking solution containing the primary antibody. Then, a 20 min washing in PBS with 0.1% Tween20 (Sigma-Aldrich, #P1379) was performed, followed by incubation for 1 hour at room temperature in a blocking solution containing the secondary antibody. Finally, tissues were washed in PBS with 0.1% Tween20. Slides were mounted using a fluorescence mounting medium (Dako Agilent Pathology Solutions, #S3023) and covered with a cover slip (Menzel-Gläser, #631-0853 VWR).

### Rnascope and In Situ Hybridization Chain Reaction (HCR)

RNA-scope was performed with the ACDBio (https://acdbio.com) Manual assay kit using RNAscope Probe-hs-TBX1-C2 (Target region: 100-769) and RNAscope Probe-hs-HOXB1-C2 (Target region: 528-2015) according to the manufacturer's instructions. RNAscope Probe-hs-PPIB-C1 was used as a positive control. The probes were designed and manufactured by ACDBio.

HCR fluorescent in situ was carried out using the HCR kit (v. 3), purchased from Molecular Instruments (molecularinstruments.org), according to the manufacturer's instructions with the slight modification of adding 100 μg/ml salmon sperm DNA to the pre-amplification solution and the amplification solution including the hairpins to reduce nonspecific binding. The HCR probe WNT5A (B3) was designed and manufactured by Molecular Instruments.

### Image Acquisition and Analysis

Spinning disk confocal microscopes (Olympus spinning disk system based on an IX3 Series (IX83) inverted microscope, equipped with a Yokogawa W1 spinning disc) were used to image fixed tissue sections. Live imaging was carried out using an inverted widefield microscope for brightfield and fluorescence (Axioobserver Z1 equipped with an sCMOS camera (Hamamatsu Orca Flash 4). Cardioids in 96-well plates were also imaged using a Celigo Imaging Cytometer microscope (Nexcelom Biosciences, LLC).

### Flow Cytometry

Cardioids (8 cardioids per condition) were dissociated using a 1.5 mL CM dissociation medium (Stem Cell Technologies, #05025) for 7-10 min at 37° C. Dissociation of CMs was stopped by adding 7.5 ml of the support medium. After centrifugation for 4 min at 130 g, cells were resuspended in 600 ul PBS with 0.5 mM EDTA (Biological Industries, #01-862-1B) and 10% FBS (PAA Laboratories, #A15-108). Cells were acquired with a FACS LSR Fortessa II (BD) and analyzed with FlowJo V10 (FlowJo, LLC) software. FACS sorting was performed using a Sony SH800 Cell Sorter (Sony Bio-technology).

### Rna Extraction and Bulk RNA-Seq Preparation and Analysis

RNA was isolated using an in-house RNA bead isolation kit semi-automated using KingFisher devices (KingFisher Duo Prime). Using the QuantSeq 30 mRNA-Seq Library Prep Kit FWD (Lexogen GmbH, #015), the bulk RNA-seq libraries (N=3, n=8) were generated according to the manufacturer's instructions. After the preparation of the libraries, samples were checked for an adequate size distribution with a fragment analyzer (Advanced Analytical Technologies, Inc). Then the RNA-seq library was submitted to the Vienna Biocenter Core Facilities (VBCF) Next-Generation-Sequencing (NGS) facility for sequencing. Reads were preprocessed using umi2index (Lexogen) to add the UMI sequence to the read identifier, and trimmed using BBDuk v38.06 (ref=polyA. fa.gz, truseq. fa.gz k=13 ktrim=r useshortkmers=t mink=5 qtrim=r trimq=10 min length=20). Reads mapping to abundant sequences included in the iGenomes NCBI GRCh38 references were removed using bowtie2 v2.3.4.1 alignment. The remaining reads were analyzed using genome and gene annotation for the GRCh38 assembly obtained from Homo sapiens Ensembl release 94. Reads were aligned to the genome using star v2.6.0c, and reads in genes were counted with featureCounts (subread v1.6.2) using strand-specific read counting (−s 1). Differential gene expression analysis on raw counts, and principal component analysis on variance stabilized, transformed count data were performed using DESeq2 v1.18.1.

### Real-Time Quantitative Polymerase Chain Reaction

The isolated RNA was reverse transcribed to cDNA using the Reverse Transcription Kit (Invitrogen, #18080044) with a C100 Touch Bio-Rad Thermal Cycler. Quantitative PCR was performed using the GoTaq qPCR master mix 2× (Promega, #A6001) with a Bio-Rad CFX384 Real-Time thermal cycler. Values of gene expression of each sample were obtained in triplicates. The Log-fold change of the sample from PBGD as a housekeeping gene and a pluripotent stem cell sample for normalization were used.

### Contraction Analysis

Cardioids were fed fresh CDMI media 1-2 hours before recordings. The 96-well plate was placed in an environmentally controlled stage incubator (37° C., 5% CO2, water-saturated air atmosphere, Okolab Inc, Burlingame, CA, USA). Each well was imaged using widefield phase-contrast microscopy (Axioobserver Z1 (inverted) with sCMOS camera, Zeis) at 100 frames per second for 30-60 seconds. Videos were then analyzed using MUSCLEMOTION; the data was read into a custom-made software for reported calculations. Percent beating was defined by if the cardioid beat once within the entirety of the recording. Beats per minute were calculated by counting the total number of beats in the video, dividing them by the length of the video in seconds, and multiplying by 60. The extent of contraction is the amplitude given from MUSCLEMOTION divided by the size of the cardioid.

### Calcium Transients

To generate a WTC line expressing the GCaMP6f gene, an AAVS1-integrating construct with a CAG promoter followed by the GCaMP6f sequence was chosen (Mandegar et al. (2016) Cell Stem Cell 18, 541-553) and introduced as previously described (Hofbauer, et al. (2021b) Cell 184, 3299-3317.e22).

Cardioids were differentiated into LV, RV, atrial, OFT and AVC or multi-chamber cardioids using the protocol above. Cardioids were fed fresh CDM-I media 1-2 hours before recordings. The 96-well plate was placed in an environmentally controlled stage incubator (37° C., 5% CO2, water-saturated air atmosphere, Okolab Inc, Burlingame, CA, USA). Each well was imaged using widefield microscopy (Axioobserver Z1 (inverted) with sCMOS camera, Zeis) at 50-100 (optimally 50) frames per second for 30-60 seconds. Cardioids were excited at 470±10 nm using a light-emitting diode (LED). Analysis of signal propagation: The peaks were identified using the whole cardioid analysis pipeline. Only pixels with a maximum intensity higher than an organoid-specific threshold were considered. The intensity was calculated per pixel, normalized to 1, and each trace smoothed using a rolling average over 3 frames. The frame at which a pixel reached 50% of peak intensity was recorded. The first frame in which more than 30 pixels reach 50% max intensity is defined as the first frame. The last frame in which all besides at most 30 pixels reach 50% max intensity is defined as the last frame. The average position of the biggest connected component of pixels which reached 50% of peak intensity first, is considered the origin of signal propagation. The speed of signal propagation is then calculated for all of the other pixels by dividing the distance between the pixel and the origin and the frame difference between the frame where the pixel reaches 50% of peak intensity and the origin frame. The speeds are all averaged together for every pixel and across all beats to determine the speed of signal propagation in the organoid. Images of signal propagation are made using the same technique, and each pixel is color-coded based on frame difference. Cardioids were excluded from this analysis for 4 reasons (1) the cardioid does not beat, (2) there is no clear directionality, (3) there are 2 origins (4) if less than 10% of the cardioid is expressing reporter protein.

### Multiple Electrode Array (MEA):

MEA was used to perform the electrophysiological recordings of the extracellular field potential. BioCAM Duplex (3 Brain) along with a single-well Accura MEA chip (3Brain) were employed. The MEA chip consists of 4096 gold-coated electrodes, with a pitch of 60 um, covering an area of 3.8×3.8 mm.

MEA chip reservoir was rinsed with 70% ethanol to sterilise, followed by 4 washed with Mili-Q water. Then, PBS was added and chips were left overnight with PBS to enhance connectivity. Next, PBS was removed without complete drying and cardioids on day 9.6 for single cardioids and between day 12-15 for multi-chamber cardioids of differentiation were carefully placed at the centre of the MEA chip using 200 ul wide-bore pipette tips (Thermofisher #2069G). To secure their position and maximise the contact area between the cardioids and the chip, a membrane and a homemade anchore were placed on top of the cardioids. Finally, 1.5 mL of CDM-I was added to the reservoir, and the MEA chips were kept overnight at 5% CO₂ incubator at 37° C. to further improve connectivity.

Recordings were conducted using BrainWave 4 software, using cardiac organoid presettings. Recordings were performed at 37° C. and the entire chip was covered with a black lid to prevent light exposure. Field potential signals from beating cardioids were acquired through a 5 Hz high-pass filter, and 1.1 electrode was used as a reference electrode. The stability of the wave-forms was confirmed for a period of 5 to 10 minutes to ensure consistency before a 5 min recording.

### Patch-Clamp Recordings of Single Cardiomyocytes

Cardioids were dissociated using the STEMdiff Cardiomyocyte Dissociation Kit (Stem Cell Technologies, #05025) according to the manufacturer's protocol (incubated for 10-20 minutes at 37° C. to thoroughly dissociate organoids) and subsequently seeded at low densities of 15-40 k cells in Laminin-511 E8 Fragment (AMS-BIO, #AMS.892 011, 0.5 μg/cm²) coated 35 mm tissue culture-treated dishes (Corning, #430165).

Cells were maintained at 37° C. in a humidified incubator with 5% CO₂, and whole-cell patch-clamp experiments were performed on single beating cardiomyocytes 4-13 days post-plating. Glass micropipettes with resistances of 1.5-4 MΩ were pulled from glass capillaries (Harvard Apparatus, #BS4 64-0792) using a Sutter P-1000 Micropipette Puller (Sutter Instrument). The extracellular solution consisted of the following (in mM): 140 NaCl, 5.4 KCl, 2 CaCl₂), 1 MgCl₂, 5 glucose, and 10 HEPES, with pH adjusted to 7.4 using NaOH. The intracellular pipette solution contained the following (in mM): 150 KCl, 5 NaCl, 2 CaCl₂), 5 EGTA, 10 HEPES, and 5 MgATP, with pH adjusted to 7.2 using KOH. Data was acquired at 10 kHz and low pass filtered at 2.9 kHz using a HEKA EPC 10 USB Quadro (HEKA Elektronik GmbH) employing PATCHMASTER NEXT software (HEKA Elektronik GmbH). Spontaneous electrical activity was recorded in current-clamp mode and analyzed using custom-made MATLAB (MathWorks) software. Action potential amplitudes were measured from peak to maximum diastolic potential, and APD values were calculated from action potential peak to the respective percentage of the amplitude's repolarization. Parameters were individually calculated for 15-20 consecutive action potentials per cell and then averaged.

### Optical Action Potentials

Cardioids were dissociated the same way for patch-clamp experiments, see the previous section, and seeded at 40 k cells per well into 96 well-plate (Greiner Bio-One, #655182) wells previously coated with Laminin-511 E8 Fragment (AMSBIO, #AMS. 892 011, 0.5 μg/cm²). Cells were kept at 37° C. in a humidified incubator with 5% CO² for 7 to 11 days, and the medium was exchanged every two to three days.

CM monolayers were loaded with 0.7 times the manufacturer's suggested amount of the voltage-sensitive dye Fluovolt (FluoVolt™ Membrane Potential Kit (Thermo Fisher Scientific, #F10488)) after three repeated wash steps with Hank's Balanced Salt Solution (HBSS, Gibco, #14175-053). Loading was performed at room temperature for 30 minutes, after which the cells were washed with HBSS three more times. The 96-well plate was then placed in an environmentally controlled stage incubator (37° C., water-saturated air atmosphere, Okolab Inc, Burlingame, CA, USA), and fluorescence signals were recorded at an excitation wavelength of 470±10 nm using a light-emitting diode (LED), and emitted light was collected by a photomultiplier (PMT, Cairn Research Ltd. Kent, UK). Fluorescence signals were digitized at 10 kHz. 20 s recordings were subsequently analyzed offline using a custom-made MATLAB (MathWorks) software. APDs were measured at 30%, 50% and 90% repolarization. APD values were calculated from the action potential peak to the respective percentage of the amplitude's repolarization. Parameters were individually calculated for all recorded action potentials per well and then averaged. The number of analyzed action potentials per well typically ranged between 5 and 20.

### Example 3: Derivation of Cardioids from aSHF and pSHF Progenitors

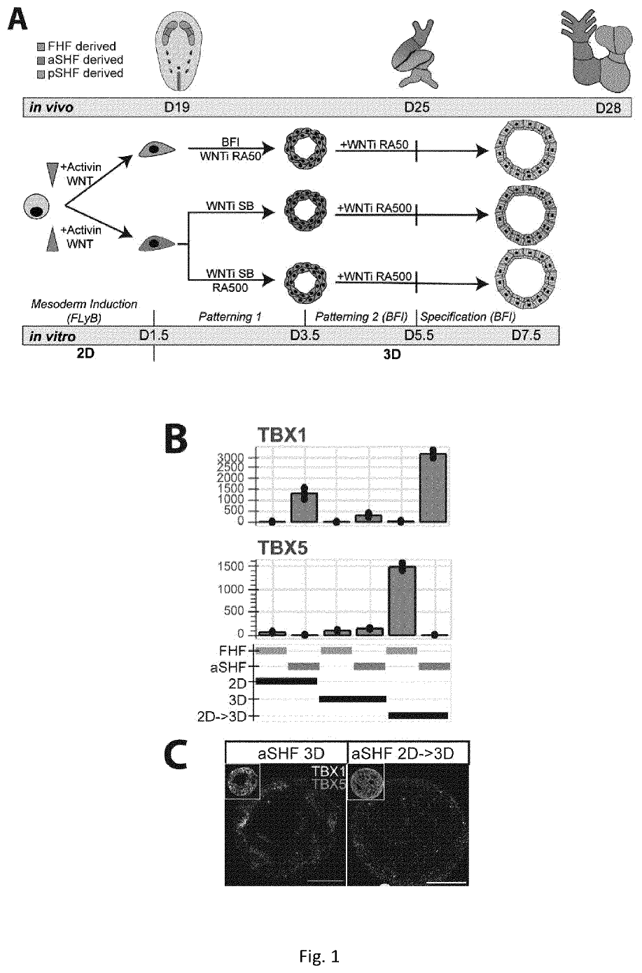

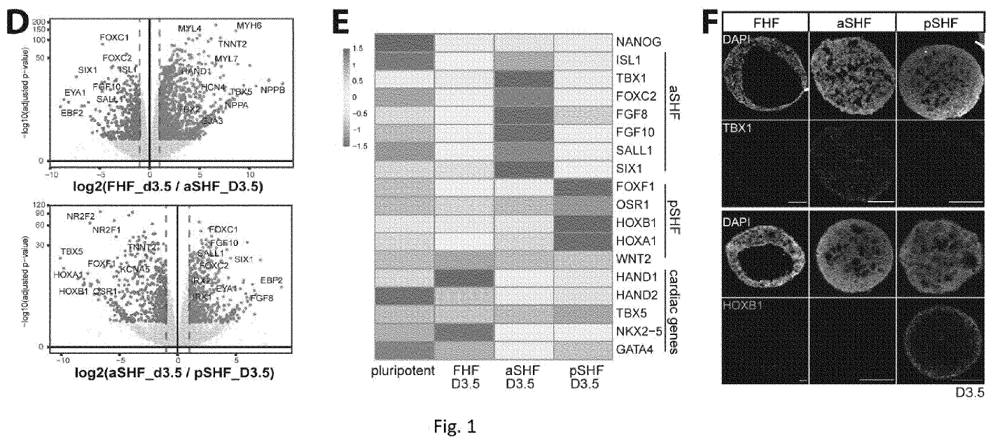

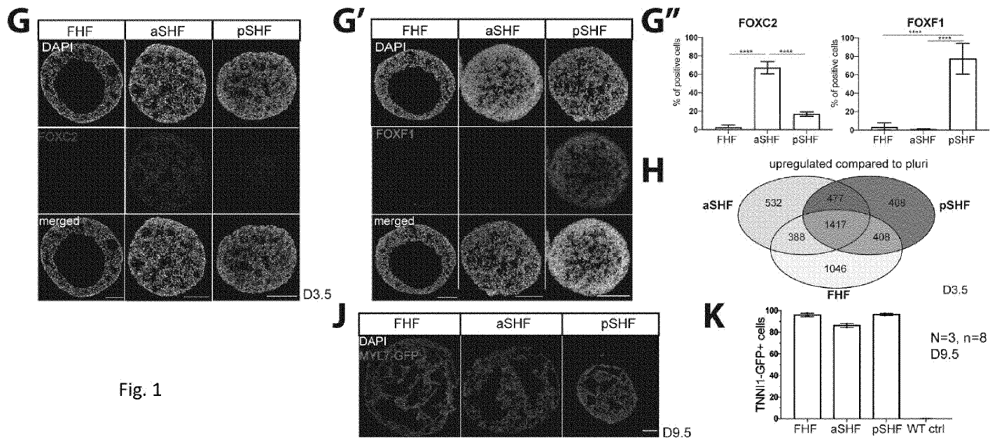

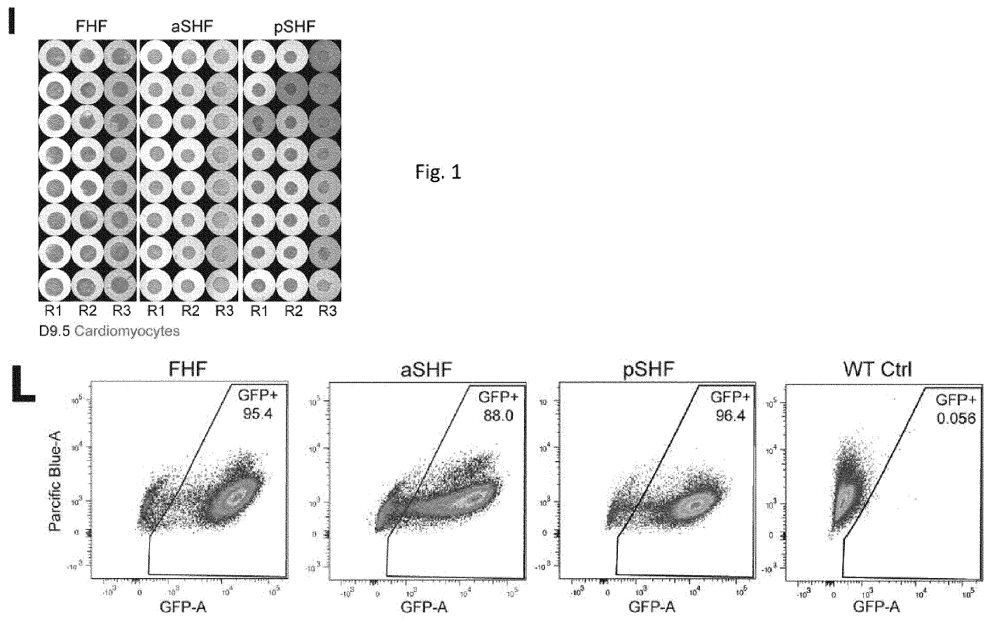

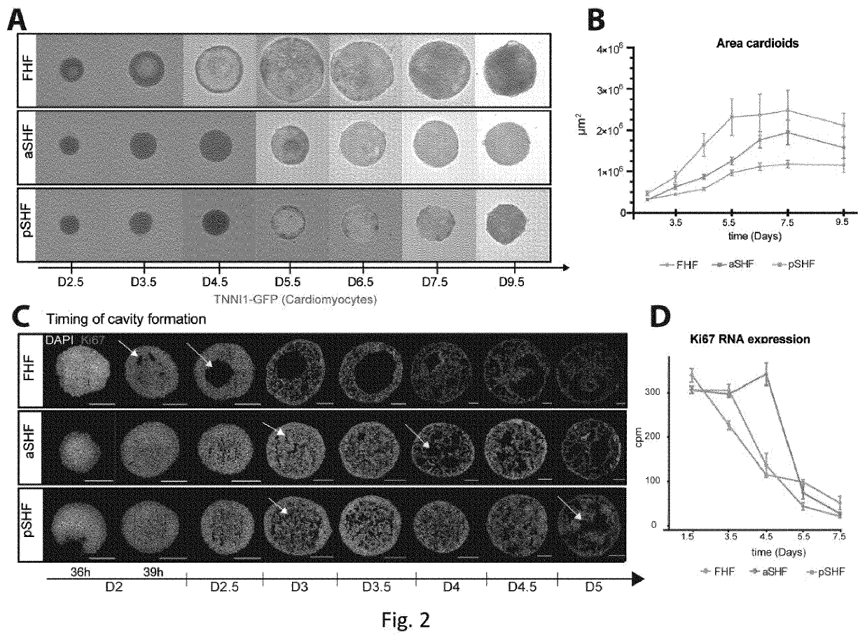

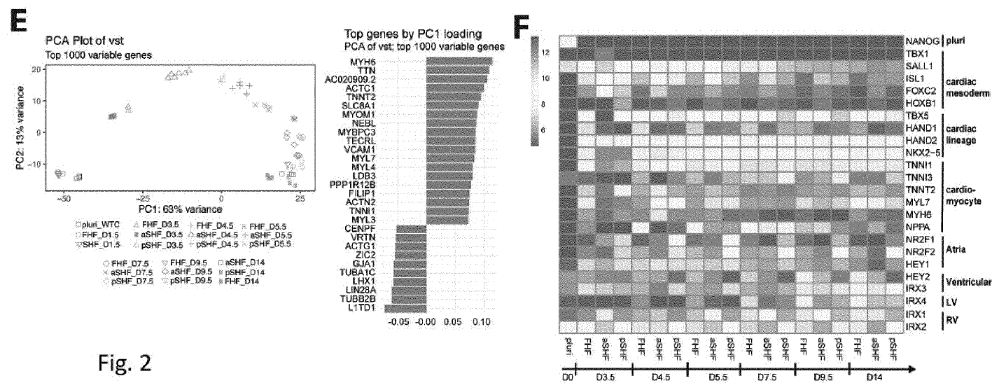

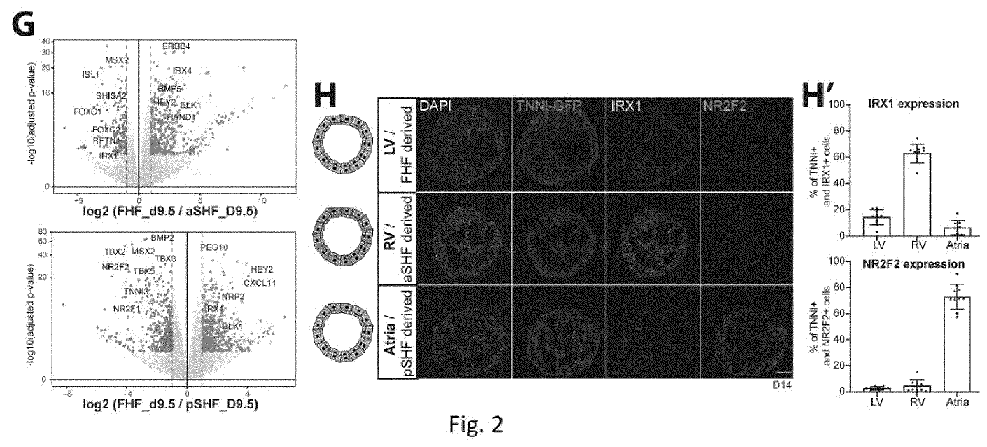

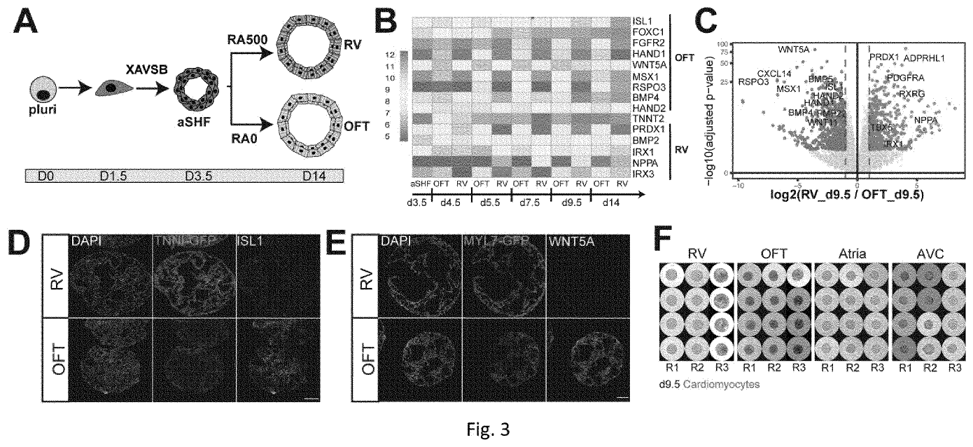

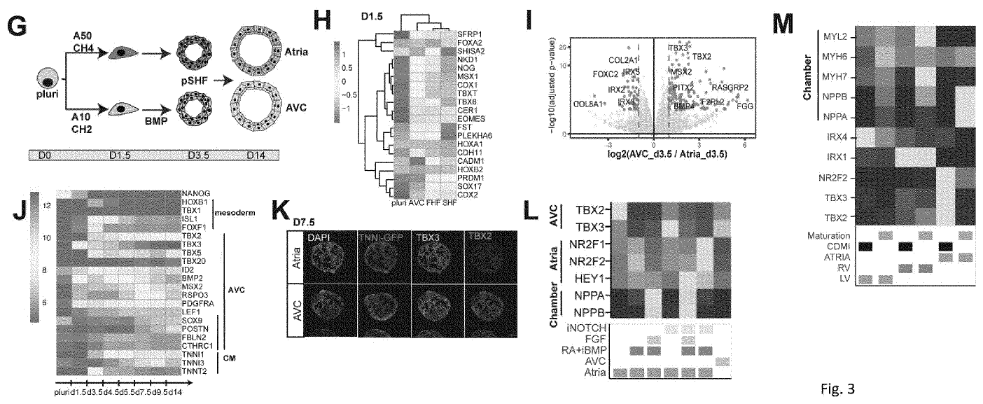

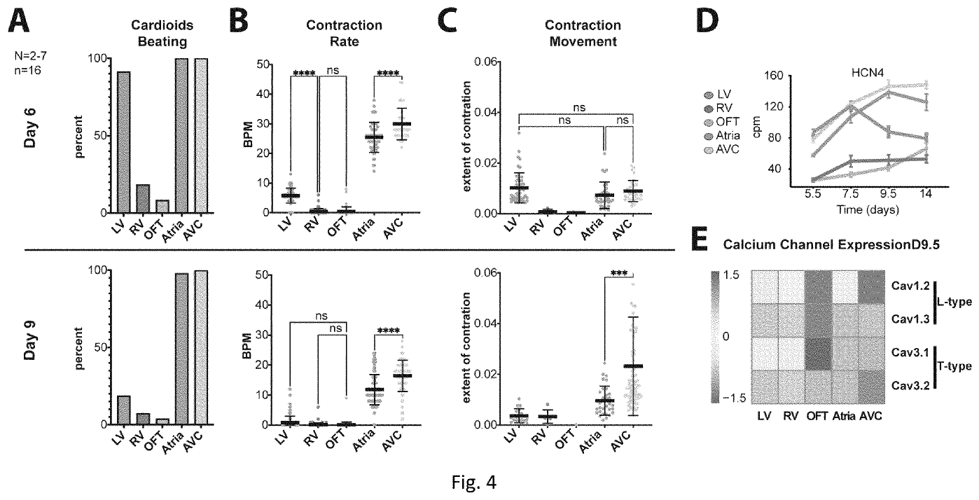

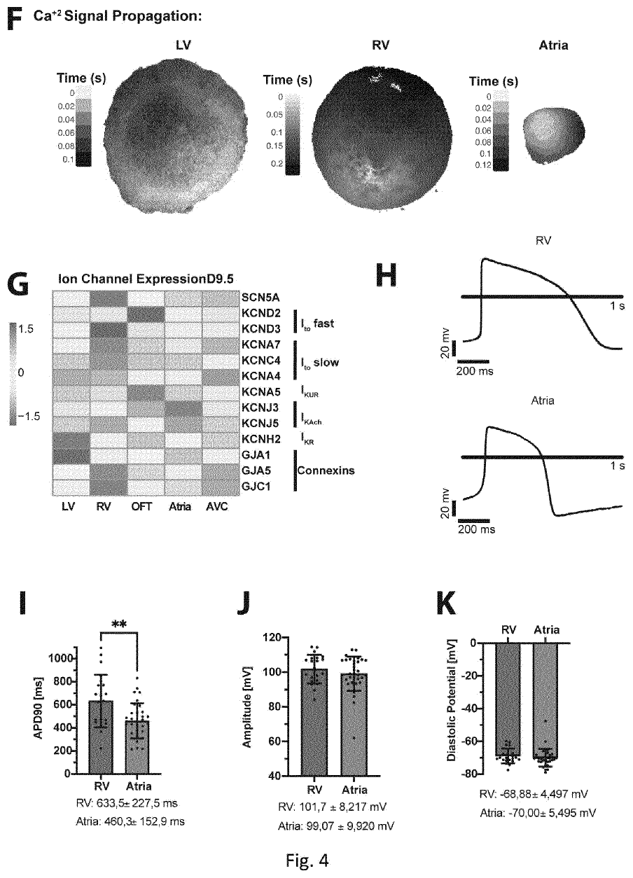

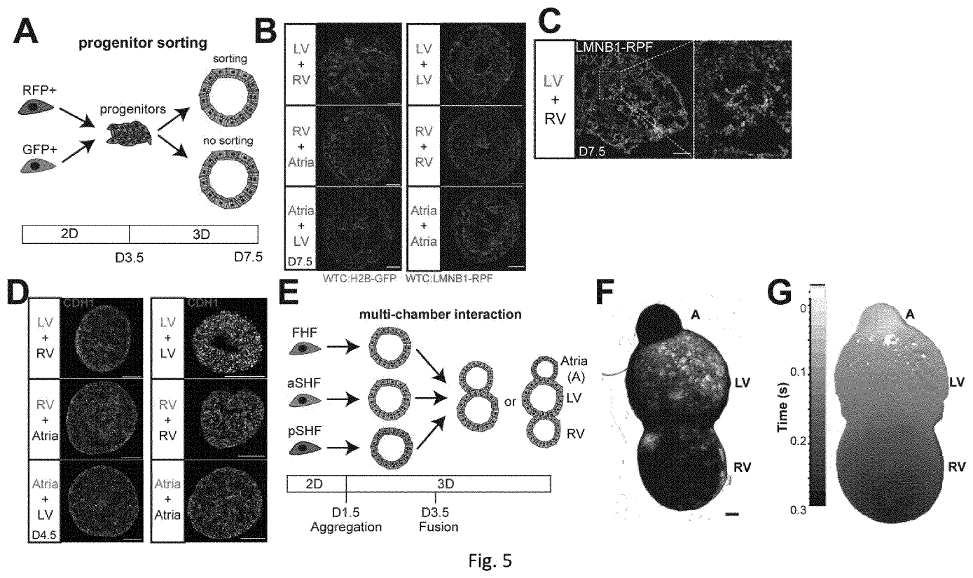

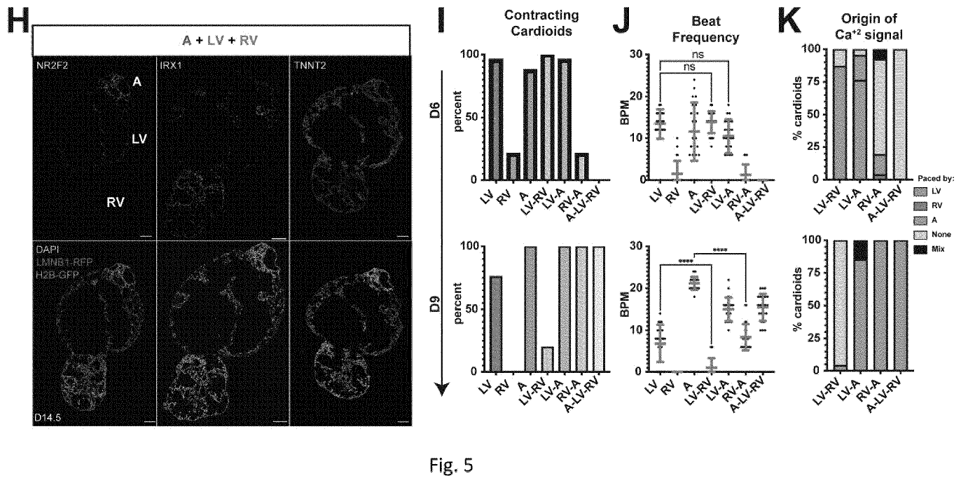

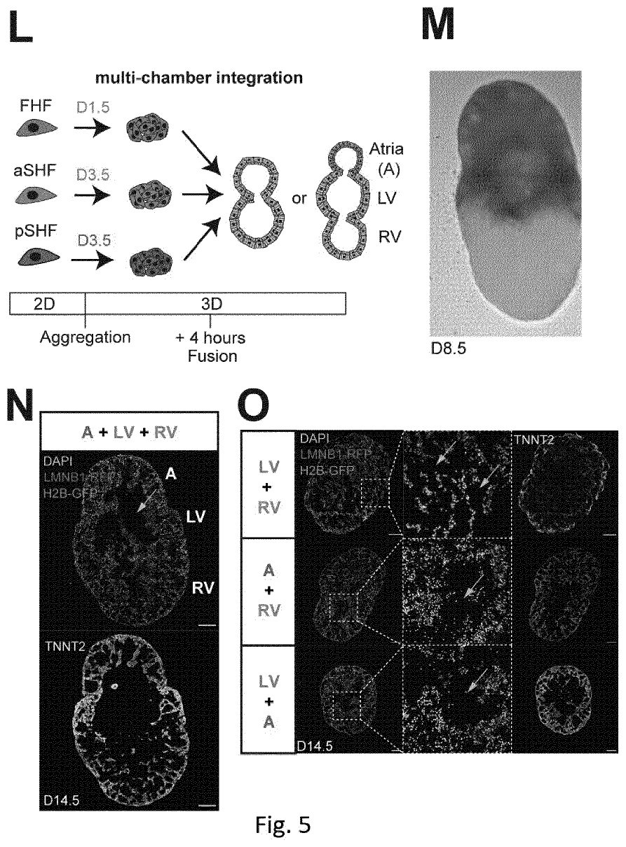

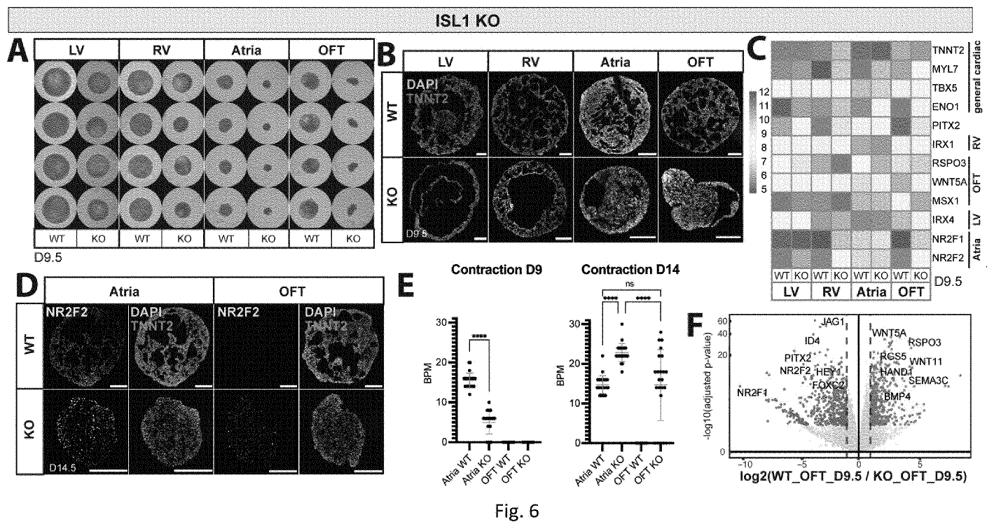

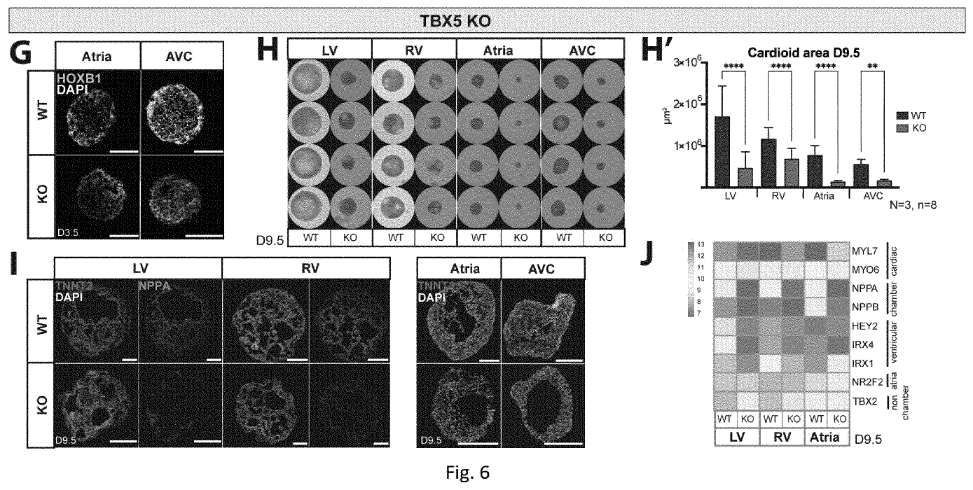

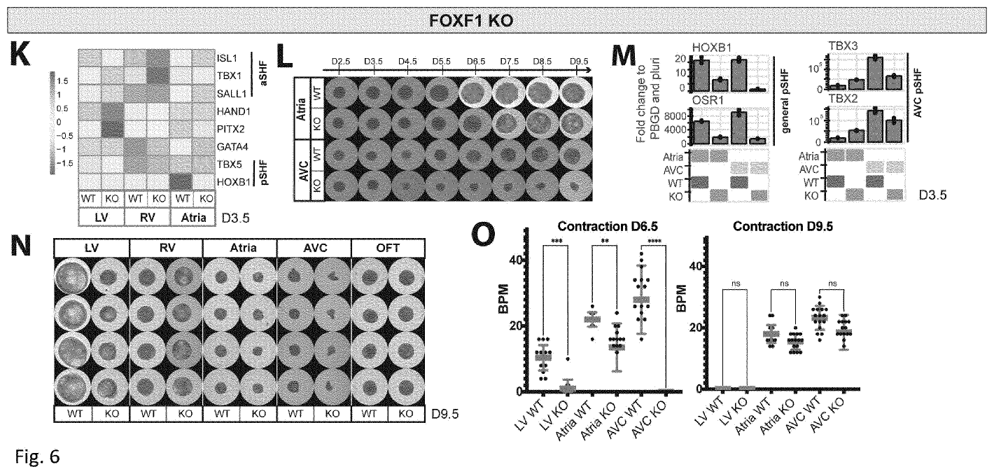

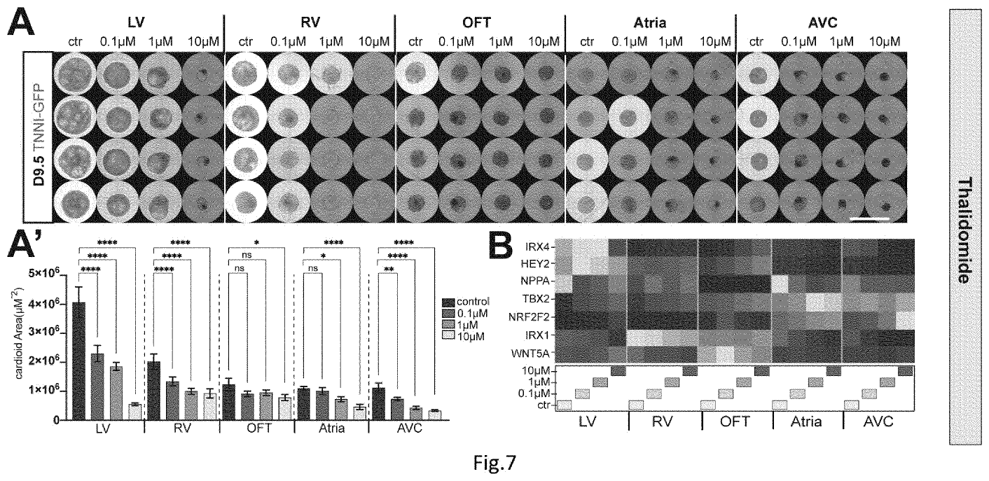

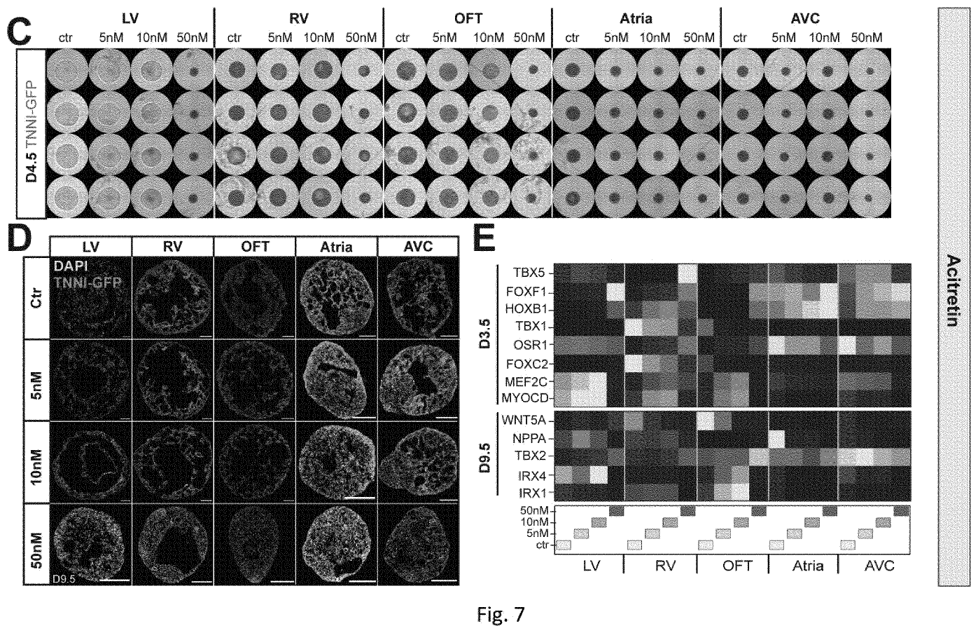

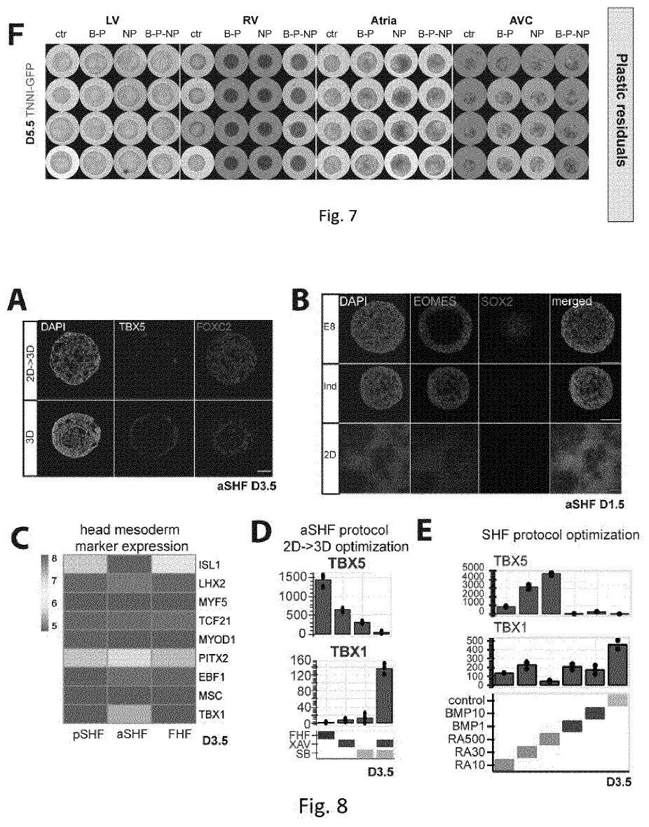

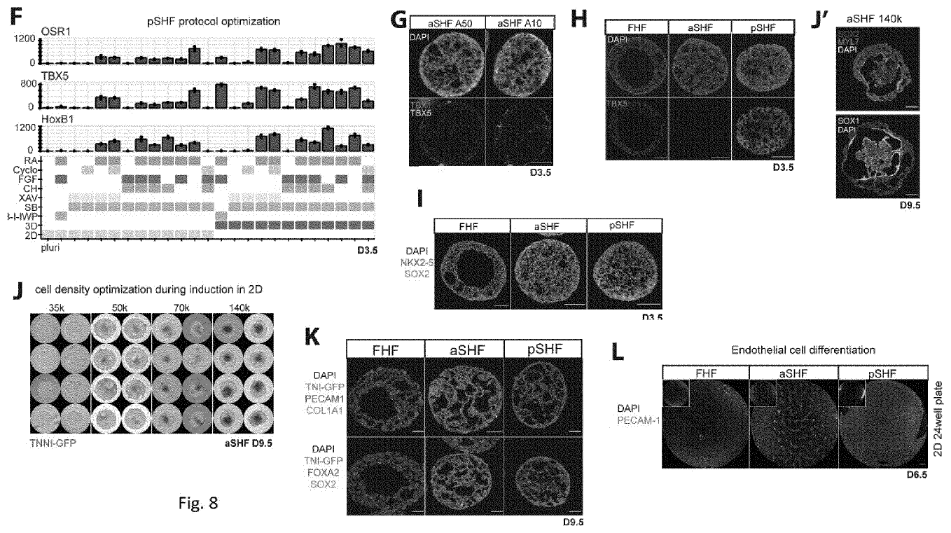

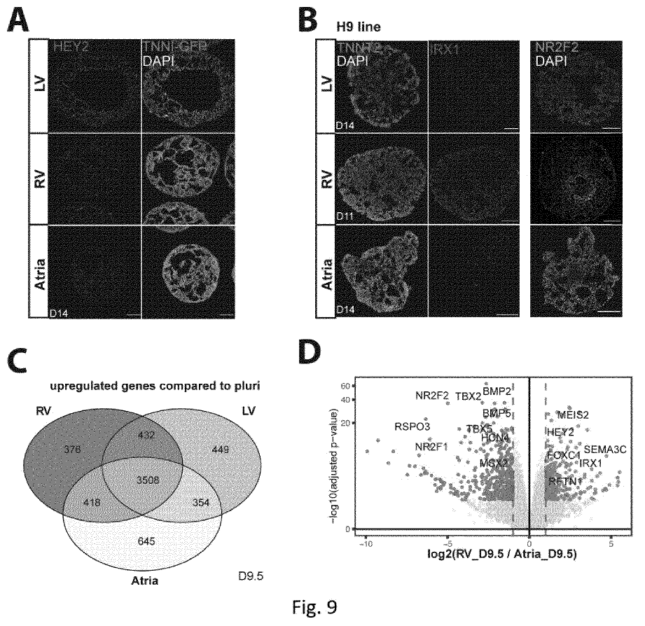

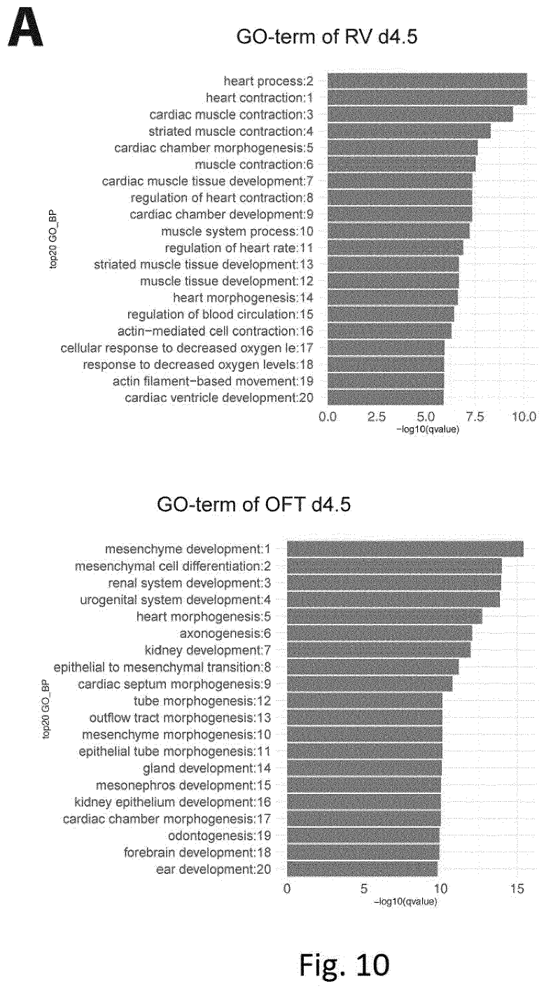

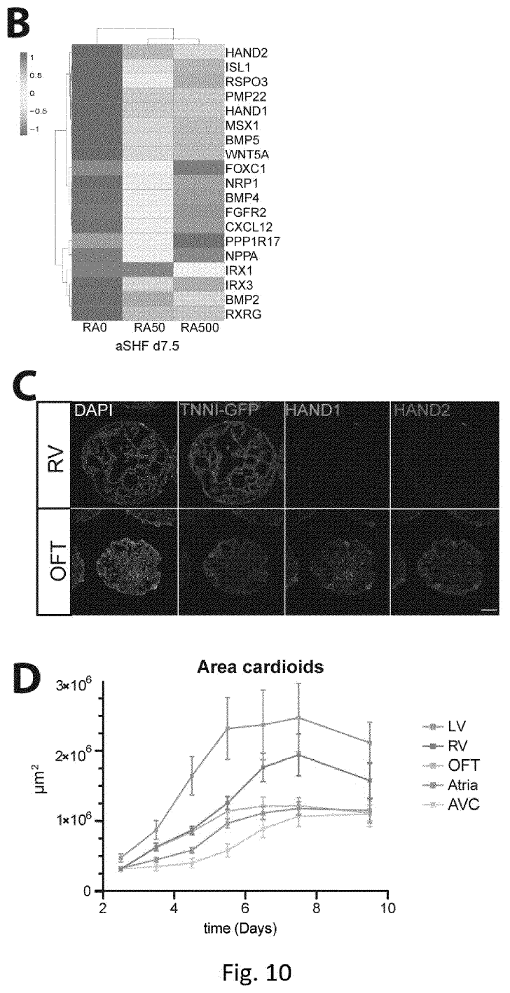

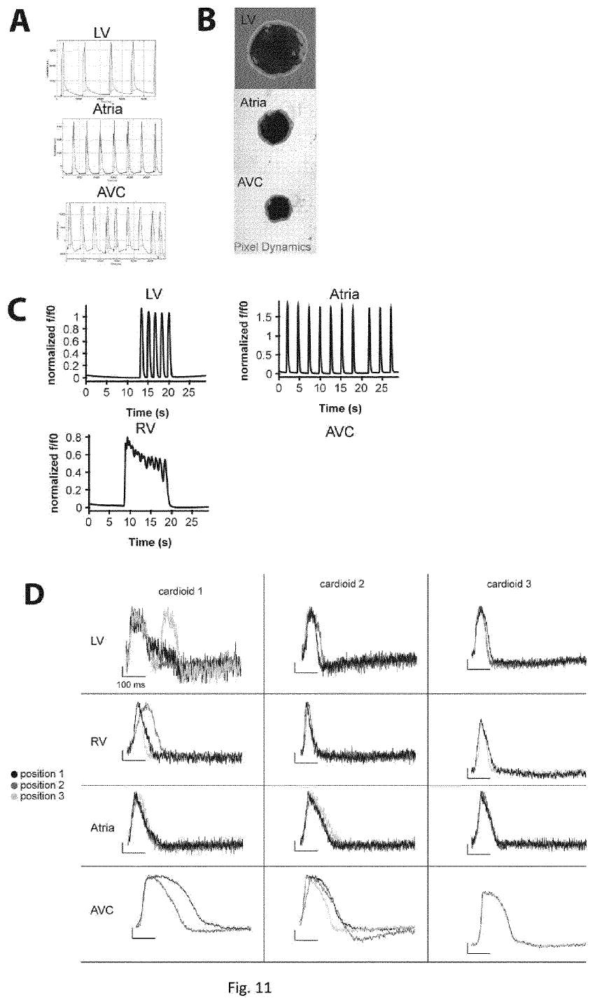

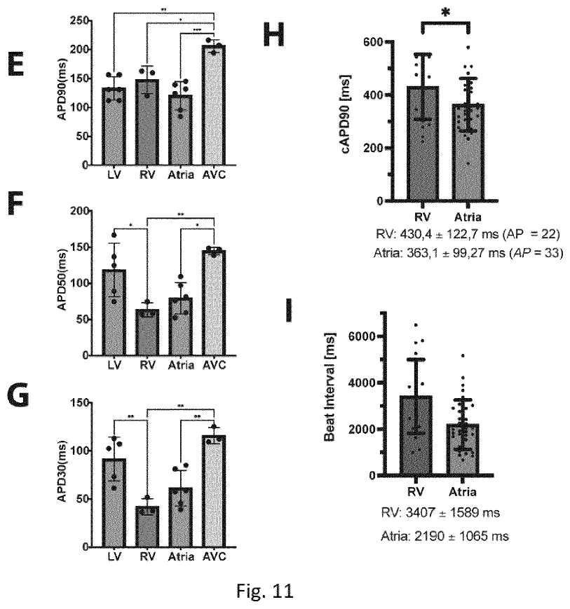

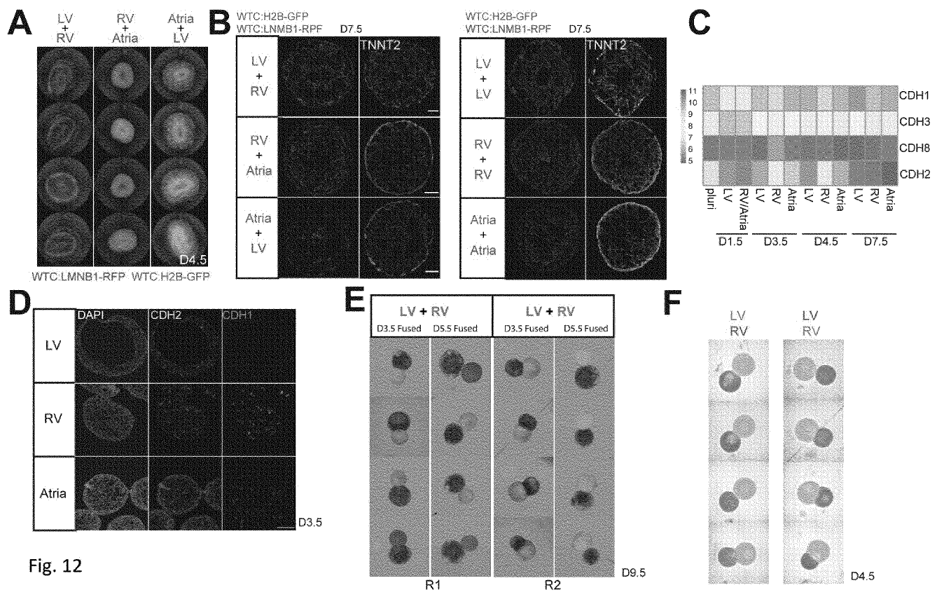

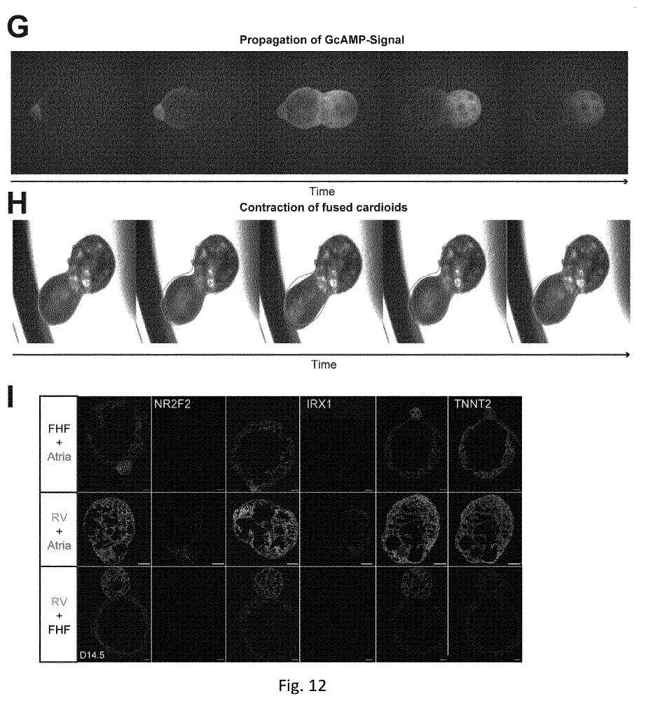

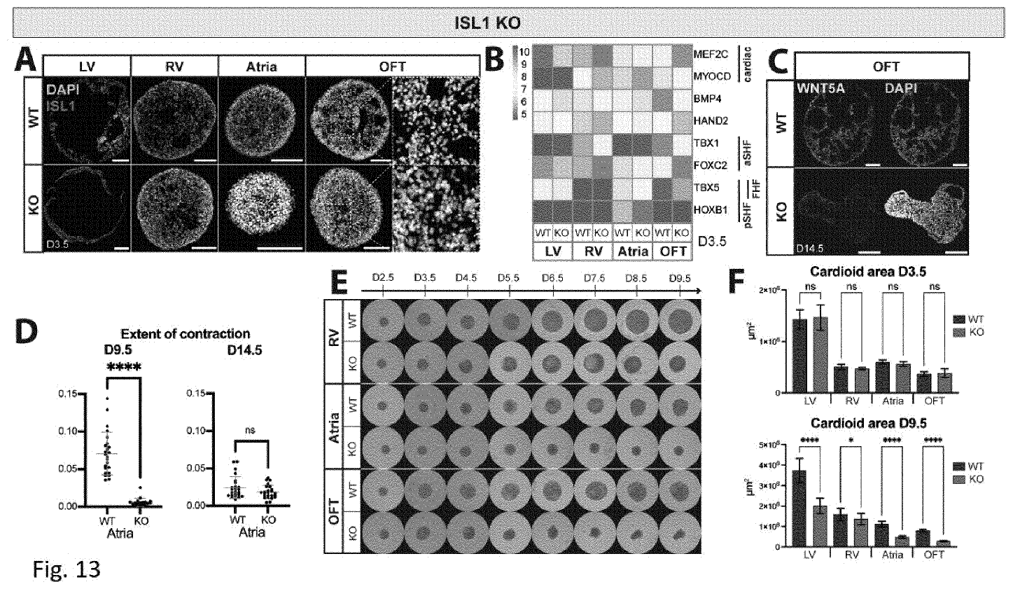

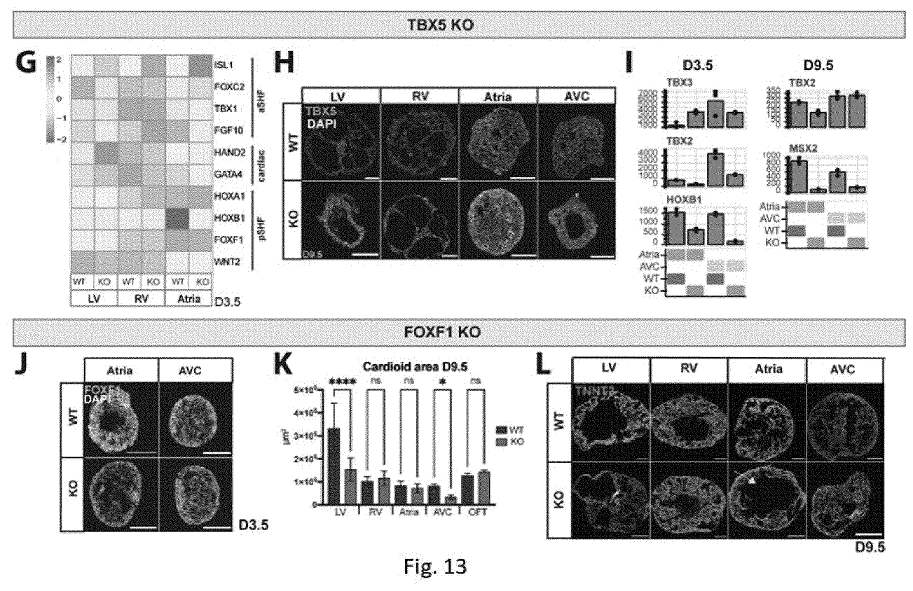

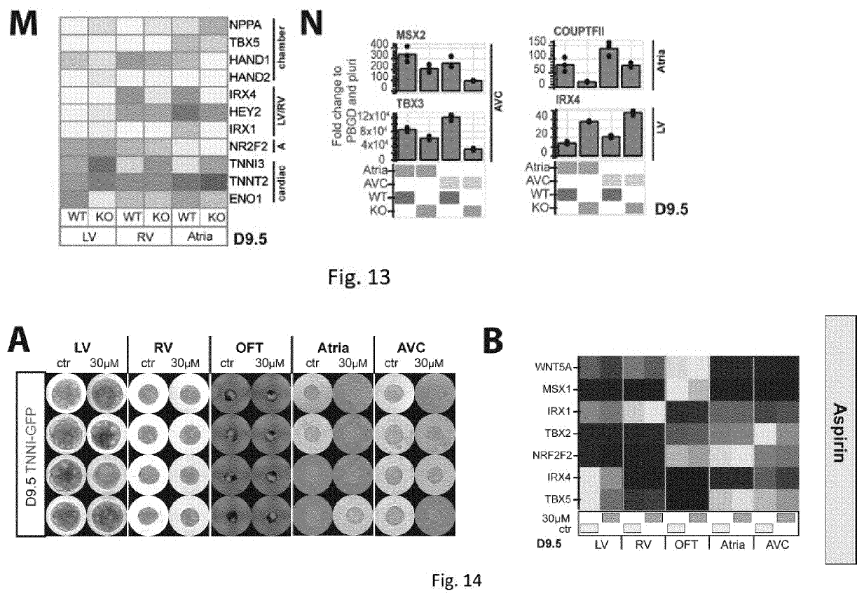

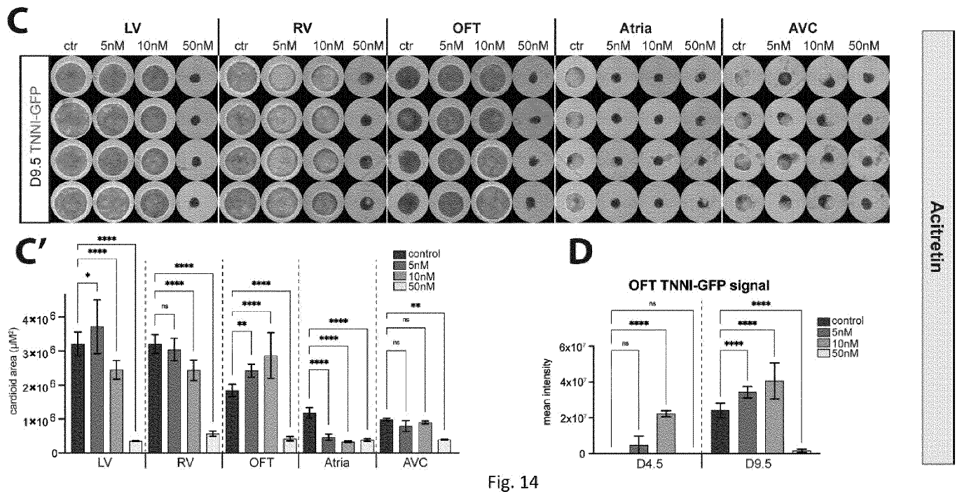

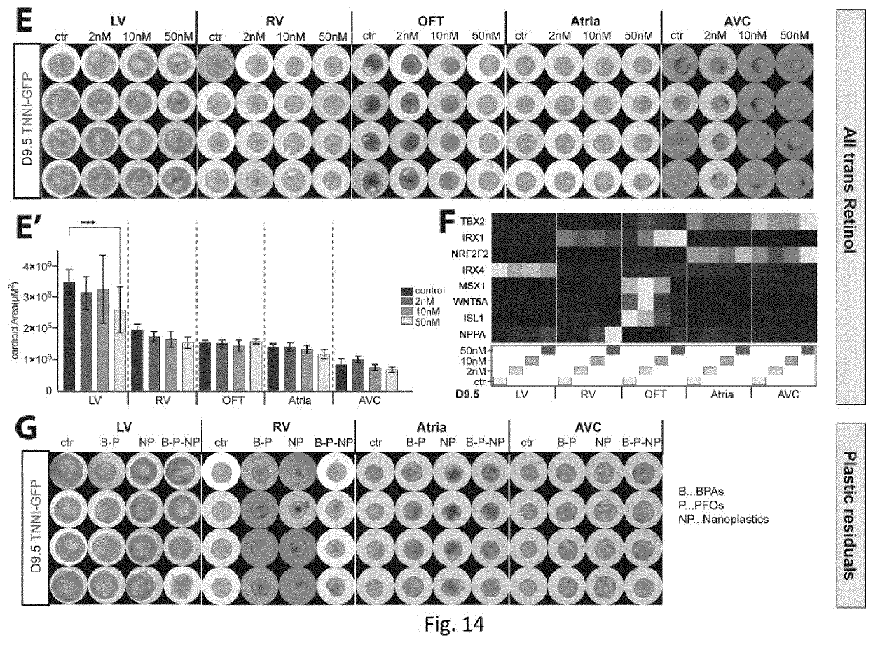

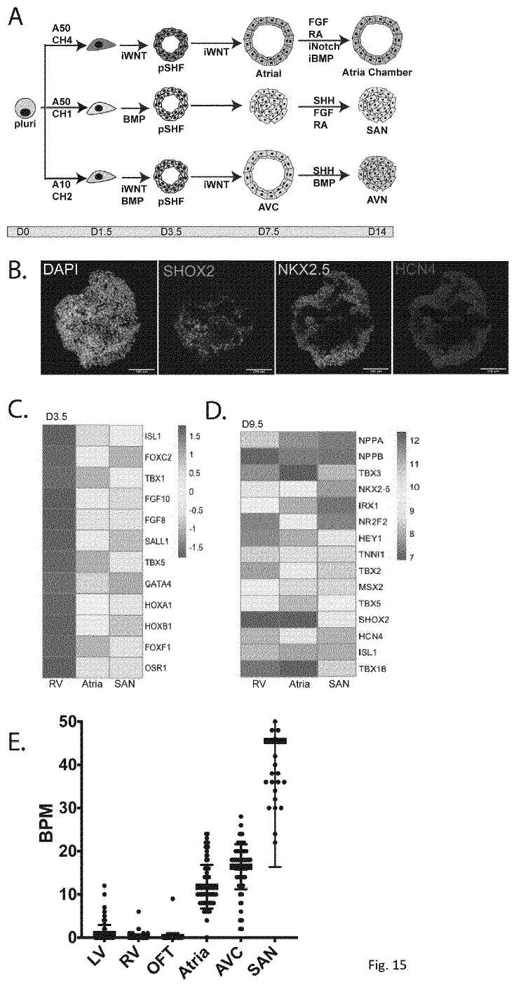

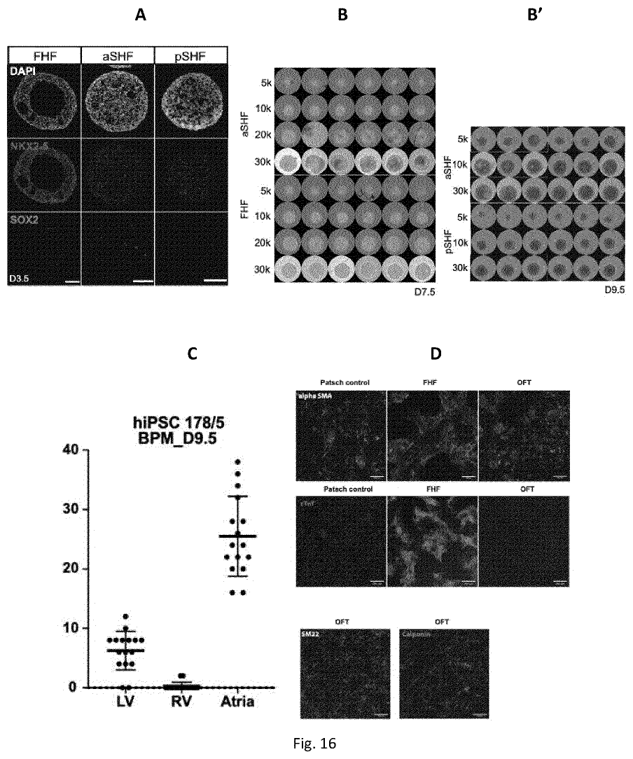

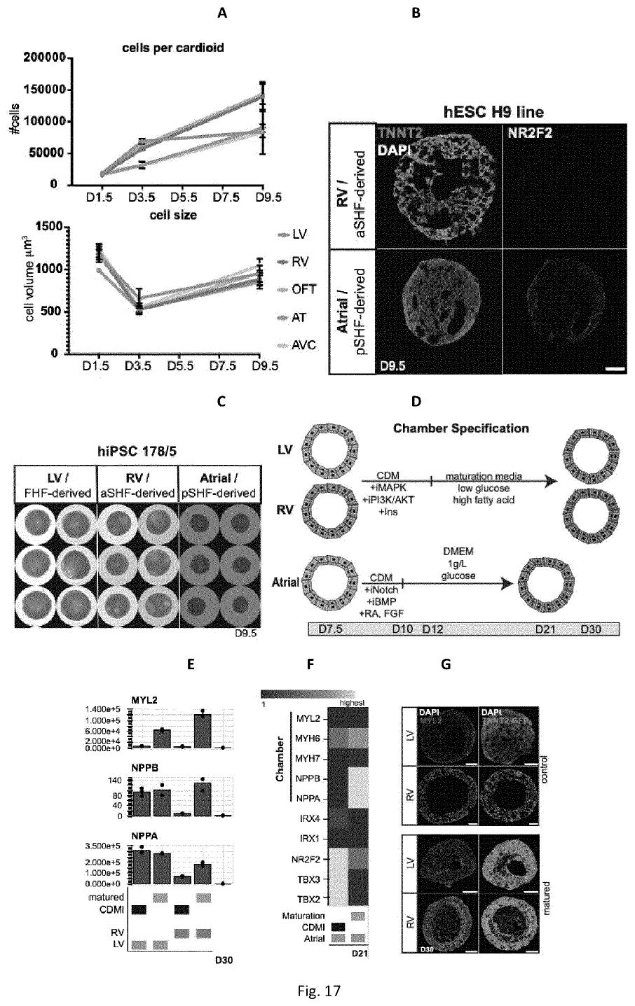

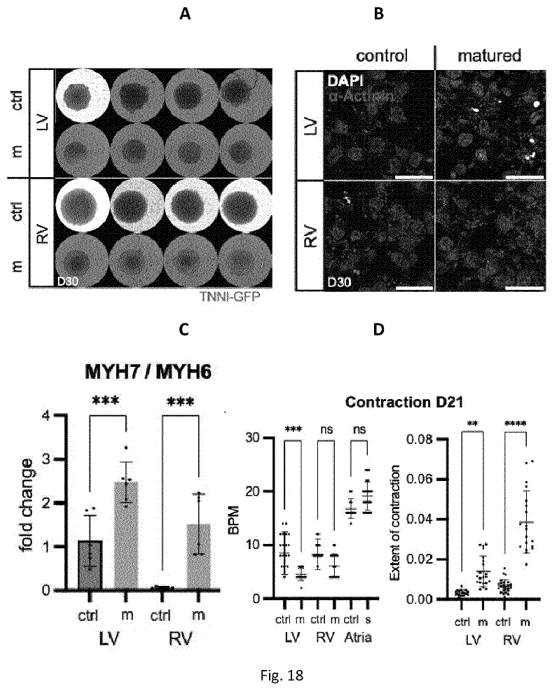

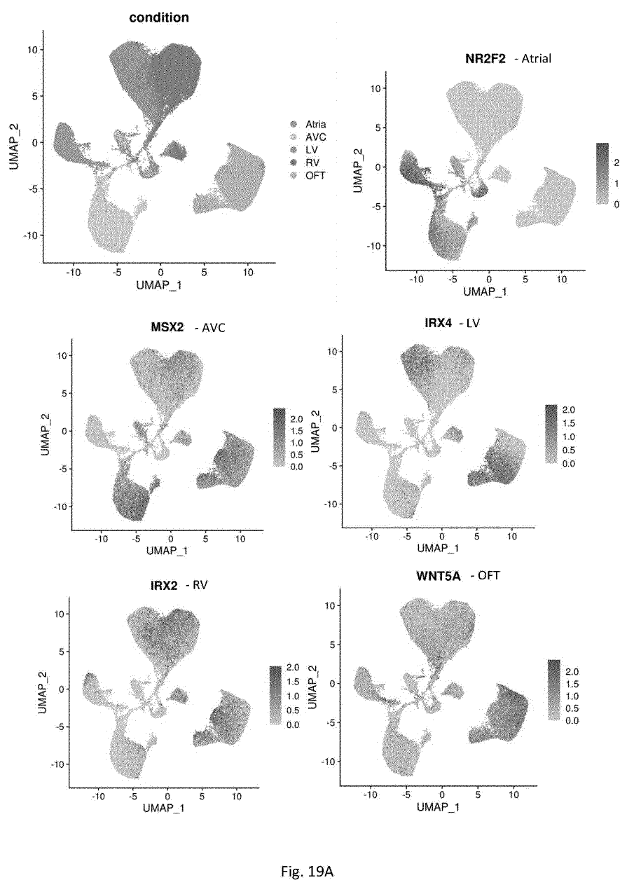

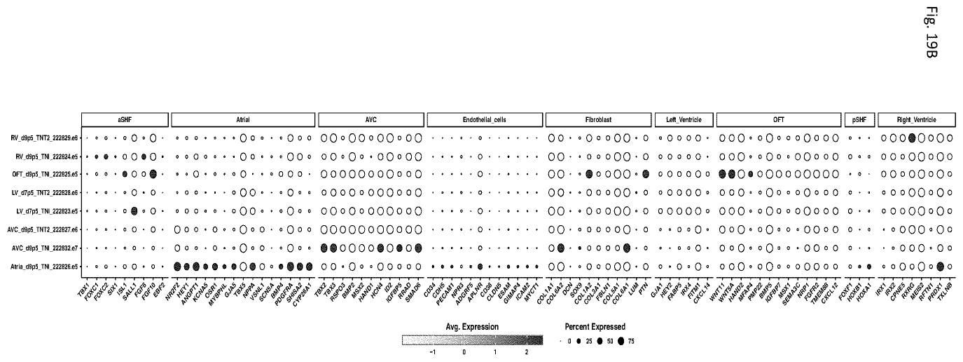

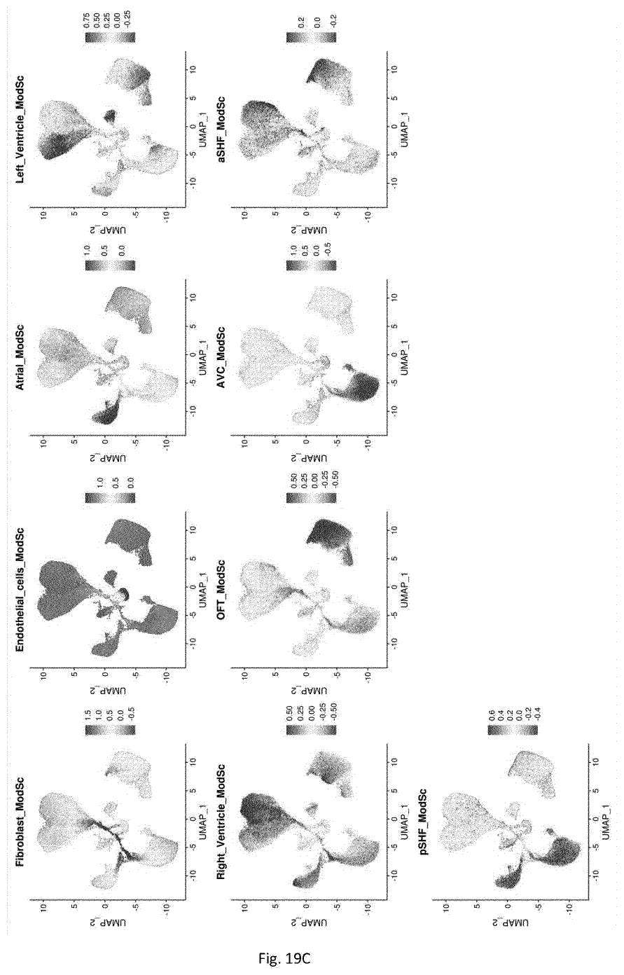

We hypothesized that the aSHF is exposed to a similar signaling environment as other anterior and dorsal embryonic regions (neuroectoderm and head mesoderm), which includes WNT and TGFb signaling inhibition. By combining these signaling conditions and the 3D cardioid approach (Hofbauer et al., Cell 2021, 184 (12): 3299-3317.e22), we aimed to develop a method to derive cardioids from the aSHF lineage. The first stage of differentiation consisted of mesoderm induction followed by the aSHF patterning stage using dual WNT and TGFb inhibition or TGF-beta signaling inhibition (FIG. 1A). After 3.5 days of 3D differentiation, we observed a heterogeneous progenitor population with one subpopulation of progenitor cells expressing the aSHF markers TBX1 or FOXC2, while another expressed the FHF and pSHF marker TBX5 (FIGS. 1B, 1C and 8A). To determine the origin of this heterogeneity, we analyzed the earlier mesoderm induction stage (day 1.5) and found that only the outside of the organoid expressed the mesoderm marker EOMES. In contrast, the organoid core still expressed the pluripotency and neural marker SOX2 (FIG. 8B), showing that mesoderm is not induced homogenously in 3D. We hypothesized that the cells in 2D would receive more equal mesoderm induction signals, resulting in a homogenous exit from pluripotency and differentiation. When we induced mesoderm in 2D and initiated the 3D differentiation only at the patterning stage (day 1.5), cells exited pluripotency efficiently (FIG. 8B), homogeneously expressed high levels of TBX1 and FOXC2, and did not express TBX5 (FIGS. 1C, 8A and 8D). In addition, we observed no expression of head mesoderm markers at day 3.5 in aSHF progenitors (FIG. 8C). Thus, the 2D->3D differentiation approach produces homogenous progenitor populations for both FHF and aSHF progenitor cells.

In contrast to the aSHF, the pSHF is exposed to retinoic acid (RA) signaling in vivo, which activates the pSHF regulators (HOXB1+, HOXA1+, TBX5+) and inhibits the aSHF expression signature (TBX1+, FOXC2+, SIX1+). Consistently, we observed that the addition of RA during the aSHF patterning stage led to a switch toward the pSHF identity (FIGS. 1F, and 8E). Manipulation of other signaling pathways (SHH, WNT, FGF, and BMP) did not influence major aSHF or pSHF markers (FIGS. 8E and 8F). In line with recent observations in vivo, different signaling levels during mesoderm induction promoted the aSHF and pSHF over the FHF lineage (FIG. 8E). We further analyzed the three progenitor subtypes by RNA-seq and strikingly, the primary markers of FHF, aSHF and pSHF were among the most differentially expressed genes (FIGS. 1D and E). Compared to pluripotency, 532 genes were specifically upregulated in aSHF, 408 in pSHF and 1046 in FHF cells, and 1417 genes were shared between the three protocols (FIG. 1H). Notably, key markers of aSHF (TBX1, SIX1, FOXC2) were lowly expressed or absent from FHF and pSHF, while pSHF markers (HOXB1, HOXA1, FOXF1) were hardly detectable or missing in the aSHF and FHF progenitors (FIGS. 1D-G″). The specificity and homogeneity of the progenitor populations were further underscored by the mutually exclusive expression of lineage-specific markers TBX1 and HOXB1 seen by RNA hybridization (FIG. 1F) and immunostaining FOXF1, TBX5, and FOXC2 seen by immunostaining (FIGS. 1G and 8H). Still, all populations are positive for the cardiac progenitor marker NKX2-5 (FIG. 8I) and mostly negative for the pluripotency and neuroectoderm marker SOX2 (FIG. 16A). Overall, by day 3.5 of the differentiation using the cardioid system, we can efficiently and homogenously generate all three major cardiac progenitors.

The FHF, aSHF and pSHF progenitors give rise to several different cardiac cell types in the embryo, including CM and endocardial cells. We showed previously that the FHF progenitors generate chamber-like contracting cardioids, which contain CMs and endocardial-like cells (Hofbauer et al., above). Following this method, we treated the a/pSHF progenitors with BMP, FGF, Insulin, and RA, and we inhibited WNT signaling (cardiac patterning 2) (FIG. 1A), resulting in the reproducible formation of contracting cardioids that contained cavities (FIGS. 1I and 1J). In contrast to FHF-derived cardioids, the a/pSHF-derived cardioids required a higher RA dosage at this stage. Efficient a/pSHF differentiation depended on a lower seeding density (FIG. 8J), as a high density led to an inefficient CM differentiation with the organoid core expressing neural markers (FIGS. 8J′). A balanced level of WNT signaling activation at mesoderm induction and seeding cell number at the cardiac patterning stage 1 helped to optimize and fine-tune cardioid formation (FIG. 8J, FIG. 16 B, B′). More than 85% of the cardioid cells expressed the key CM marker TNNI1 (FIGS. 1K and 1L), and the expression of endoderm (FOXA2), ectoderm (SOX2), and fibroblast markers (COLA1) was lacking (FIG. 8K). Finally, aSHF and pSHF cells also efficiently differentiated into PECAM1+endothelial cells in 2D when exposed to VEGF and Forskollin after the SHF patterning stage (FIG. 8L). In summary, in vivo-like signaling specifies a/pSHF progenitors differentiating into CM and endothelial lineages and allows the formation of cardioids.

### Example 4: Formation of RV and Atrial Cardioids

FHF progenitors differentiate early into CMs forming the heart tube, while aSHF progenitors proliferate first and differentiate together with pSHF progenitors at a later time point. Thus, we hypothesized that the SHF-derived cardioids would show a similar higher proliferation rate, delayed morphogenesis, and differentiation than FHF-derived cardioids. A detailed time-course analysis revealed the delayed formation of SHF cardioids (FIGS. 2A-C), a higher aSHF progenitor proliferative rate and expression of Ki67 (FIGS. 2C and 2D). The a/pSHF cardioids were smaller than FHF cardioids and started to express the CM marker TNNI1 one day later than FHF-derived cardioids (FIGS. 2A and 2B). Global gene expression confirmed that SHF-derived cardioids showed a delayed expression of sarcomeric and structural CM genes (FIGS. 2E and 2F) and delayed cavitation initiation and formation over time (FIG. 2C, white and yellow arrows). Different cardioid subtypes also showed an increase in cell number over time (FIG. 17A) and in cell size (FIG. 17C). Taken together, the developmental timing of SHF and FHF-derived cardioids is consistent with the in vivo timing of SHF and FHF differentiation and morphogenesis.

Next, we investigated whether a/pSHF-derived cardioids also followed the developmental trajectory in terms of chamber identity. In vivo, the FHF, aSHF, and pSHF give rise to the left ventricle (LV), right ventricle (RV), and atria, respectively. We tested the specification potential of a/pSHF compared to FHF progenitors by adjusting the concentration of RA. We observed that aSHF progenitors gave rise to CM with an early RV identity (IRX1+, IRX2+, IRX3+, NPPA+), while the pSHF progenitors differentiated into early atrial CMS (HEY1, NR2F1, NR2F2) (FIG. 2F). A global gene expression comparison at day 9.5 of FHF-cardioids, aSHF-cardioids and pSHF-cardioids revealed that the top differentially expressed genes include ISL1+, IRX1+, and RFTN1+ (FIGS. 2G and 9D), which have all been implicated in ventricular identity and physiology. Moreover, the comparison of FHF cardioids vs. pSHF cardioids showed upregulation of TBX5, NR2F2 and NR2F1 consistent with early atrial identity (FIGS. 2G and 9D, FIG. 17B). These results were further confirmed on a protein level for IRX1, NR2F2 and HEY2 (FIGS. 2H, 2H′ and 9A). When compared to the pluripotent state, 376 genes were specifically upregulated in RV, 645 in Atria and 449 in LV cells, and 3508 genes were shared between the three protocols (FIG. 9C). The specification of the CM sublineages was also achieved using hESC H9s (FIG. 9B, FIG. 17B) and other iPSC lines (FIG. 16C, 17C). Interestingly, we observed that the final size of pSHF-de-rived cardioids was smaller (FIGS. 2A and 2B), which is consistent with atria being smaller compared to ventricles. In summary, aSHF progenitors specify into RV-like cardioids, and pSHF progenitors form atrial-like cardioids, showing that the early priming of progenitors is important for obtaining different chamber identities in the developing heart.

### Example 5: Specification into OFT, AVC and Chamber Cardioids

Besides the right ventricle, aSHF progenitors also differentiate into the outflow tract (OFT), which gives rise to the aortic and pulmonary valve and vessel structures. Abnormalities in these are the most frequent congenital heart defects. We next investigated at what stage signaling instructed the separation of the RV and OFT and hypothesized this occurs after aSHF specification. We observed that higher RA dosages promoted aSHF specification towards the RV chamber identity (FIG. 10B), while the absence of RA signaling promoted OFT markers (WNT11, WNT5A, ISL1, BMP4, RSPO3) in the absence of chamber markers such as NPPA (FIGS. 3A-3C and 10B). We confirmed these observations at the levels for WNT5A, ISL1, HAND1 and HAND2 (FIGS. 3D, 3E and 10C). The difference between RV and OFT lineages emerged already at day 4.5 of differentiation, showing accelerated differentiation into the RV chamber program compared to the OFT (FIG. 10A). The gene expression profile of OFT shows that the marker WNT5A already gets upregulated at day 4.5, and OFT cardioids, in general, are more mesenchymal and show a delayed differentiation and are smaller in size compared to RV cardioids (FIGS. 3F and 10D). OFT cardioids had also the potential to differentiate into smooth muscle cells (FIG. 16D). Thus, aSHF progenitors can be subsequently directed into either RV or OFT cardioid formation by the presence or absence of RA, respectively.

In vivo, pSHF-derived CMs make up most of the atria and con-tribute to the AVC, a crucial region where valves and pacemaker elements develop. The precursors of the pSHF locate in different areas in the primitive streak and will migrate out at different times. Precursors of the pSHF giving rise to the AVC migrate earlier while the atrial pSHF precursors later. Thus, we hypothesized that the mesoderm induction conditions for these two different pSHF populations will differ. Consistently, intermediate levels of Activin and WNT activation resulted in AVC-specific genes being more highly expressed at later timepoints while keeping the pSHF signature. Furthermore, after the mesoderm induction stage, three distinct populations of cells (FHF, SHE, and AVC) show an anterior to posterior gene expression pattern (FIG. 3H). Another notable difference between AVC and atrial development in vivo is the high exposure of the AVC region to BMP ligands. In agreement with this, the addition of BMP at the patterning stage upregulates early AVC markers. Finally, the combination of the optimized induction and patterning stages (FIG. 3G) drove pSHF specification towards AVC identity, as seen by RNA-seq (FIGS. 3I and 3J) and protein expression of TBX2 (FIG. 3K). Moreover, AVC cardioids were smaller than atrial cardioids over the whole time course (FIGS. 3F and 10D). Overall, the sub-specification of pSHF progenitors into atria or AVC cardioids occurs as early as the mesoderm induction stage, showing the plasticity of pSHF progenitors.

During the early stages of cardiogenesis, the atria and AVC have similar gene expression profiles. Subsequently, the atria, LV and RV start upregulating chamber gene expression programs, while the AVC and the OFT do not. To achieve chamber specification, we combined two already published CM chamber specification and maturation signaling treatment (FIG. 17D). Importantly, LV and RV cardioids upregulated the chamber markers MYL2, MYL7, NPPB and NPPA (FIG. 3M, 17E, 17G). We also observed an overall increase in TNNI expression (FIG. 18A), improved sarcomere (fishbone) structure (FIG. 18B), an increased MYH7/6 ration (FIG. 18C), and, sursprisingly, an improved contraction behavior/amplitude (FIG. 18D). However, this approach did not stimulate the atrial chamber program and suppressed atrial identity. Therefore, we performed a screen for signaling factors that specifically promote atrial chamber differentiation (FIG. 17D). We found that activation of FGF and RA and inhibition of NOTCH and BMP pathways promoted the atrial chamber gene program while down-regulating AVC-specific genes (FIG. 3L, 17F). We then combined this treatment with a low glucose medium similar to the ventricular chamber metabolic maturation treatments and observed further chamber differentiation (FIG. 3M, 17F). Overall, we have shown that we can specify and differentiate cardioids into the five major compartment identities found in the embryonic heart.

Finally, to confirm the specification into all five lineages (LV, RV, OFT, atria and AVC), we performed a single-cell RNAseq analysis in biological duplicates (FIG. 19). Using unsupervised UMAP clustering, we found that the five cardioid/cardiomyocyte subtype identities clustered separately (FIG. 19A). When we assigned well-characterized cardiac compartment markers to these clusters, they overlapped with the expected identities (FIG. 19B, 19C). These results confirmed that we developed a cardioid platform containing all the major compartment lineage identities found in the in the human heart.

### Example 6: Functional Characterization of the Five Cardioid SubTypes

The heart must function while it is developing; thus, it is imperative to also understand early cardiac function during the formation of the different embryonic heart compartments. Animal experiments suggest considerable differences in spontaneous contraction (automaticity) and beating frequency between the compartments of the heart. The FHF-derived heart tube and early LV region start to contract first and lose the automaticity as they mature. In contrast, the atrial region (developing Atria and AVC) starts to beat later, maintains the spontaneous contraction for longer, and loses the automaticity only after the cardiac pacemaker elements have formed. We hypothesized that the fused cardioid could be used to investigate these early functional developmental differences before the formation of pacemakers and before human in vivo data can be acquired.

The fused cardioids are particularly advantageous for investigating contraction dynamics using widefield microscopy. The contraction behavior (FIGS. 11A and 11B) of the compartment-specific cardioids on day 6.5 showed that 90-100% of LV, atria and AVC showed automaticity of beating and a greater extent of contraction. In contrast, only 18% of the RV cardioids spontaneously contracted as did only 8% of the OFT cardioids with a low contraction extent (FIGS. 4A and 4C). On day 9.5, atria and AVC retain automaticity, while automaticity and contraction rate reduce in LV, RV and OFT (FIGS. 4A-C). The loss of automaticity correlates with the downregulation of HCN4, encoding a potassium/sodium channel (FIG. 4D), as in vivo. Interestingly, each cardioid subtype has its distinct beat pattern; the atria and AVC beat very regularly, while the LV and RV beat in regular bursts (FIGS. 11A and 11C). Importantly, these observations were reproducible across both technical and biological replicates. To gain further insights into signal propagation in cardioids, we derived a GCaMP reporter line to trace calcium transients (FIG. 4F) . . . . Within one cardioid, the origin of signal propagation varied between beats. We also observed differences between cardioid subtypes, supported by expression differences seen in both T and L type calcium channels (FIG. 4E). Overall, each cardioid subtype has a distinct contraction and signal propagation profile.

Ion channel expression during early heart development is relatively uniform and later develops into chamber-specific expression profiles and action potential (AP) shapes for a particular species. The different cardioid subtypes initially showed a similar ion channel profile on day 3.5, which later showed differences according to their chamber specificity on day 9.5 (FIG. 4G). We used voltage-sensitive dye (FluoroVolt) imaging and patch-clamping to characterize how APs of early human CM subtypes from cardioids compare. We verified that the AP measured with FluoroVolt is consistent in different regions of the cardioid and across the cardioid subtype (FIG. 11D) with slight variation in the AVC. This could be explained by the functional heterogeneity of the AVC having the potential to develop into different structures. By comparing the AP length, we observed that the LV, RV, and atria were similar in shape, while the AVC was strikingly different (FIG. 11E-G). The RV and atrial cardioids were then dissociated to perform a patch-clamp analysis (FIG. 4H). We observed that the AP duration in atrial CM was shorter than in RV CMs, as expected from in vivo (FIGS. 4H-I and 11H-I). Furthermore, the diastolic potential was around-70 mV (FIGS. 4J and 4K), close to in vivo CMs. In addition we can measure the cardiac field potential on multiple electrode arrays (MEA). Taken together, the electro-chemical signaling of cardioid subtypes is diverse and still immature, providing a system to investigate the developmental electrophysiology of early human cardiogenesis.

### Example 7: Multi-Chamber Integration of Cardioid Subtypes

Embryonic cardiac progenitors specify in neighboring but separate areas. Upon migration of the SHF-derived RV and atrial precursors into the heart tube, they self-sort and remain separate compartments, crucial for the heart's function. Studying the molecular basis of this sorting is challenging in embryos. We hypothesized that in vitro-derived a/pSHF and FHF progenitors will have the potential to self-sort as in vivo. Indeed, when we dissociated developing cardioids of different subtypes at day 3.5 and then mixed them (FIG. 5A), we observed sorting in cardioids within 24 hours (FIG. 12A). In contrast, progenitors of the same subtype did not sort upon mixing (FIG. 5B). We observed the highest degree of sorting at day 7.5 for FHF-derived LV and aSHF-derived RV progenitors (FIGS. 5B and 5C). The sorting and pattern formation was consistent with the differential Cadherin expression signature in the different progenitors reminiscent of in vivo (FIGS. 5D, 12C and 12D). Notably, the sorted progenitors developed into TNNT2+CMs and retained the appropriate chamber identity (FIGS. 5C and 12B), confirming that the first two stages of differentiation determine lineage identity. This also implied that co-differentiation of different progenitors was possible from day 3.5 on. In summary, the multi-chamber cardioid platform can be used to further study the sorting mechanisms separating the major compartments of the heart.

In development, the LV, RV and atrial chambers co-develop; however, we are missing a multi-chamber model to study this crucial stage and process of cardiac morphogenesis. As cardiac progenitors were specified and sorted already at day 3.5, we hypothesized that co-developing cardioids would also remain separate at this stage but undergo morphogenesis together. When we placed different cardioid subtypes together on day 3.5 (FIG. 5E), they interacted efficiently (fused) after 24 hours (FIG. 12F) but kept distinct identities and compartments (FIGS. 5G and 51). In contrast, the co-development of cardioids on day 5.5 did not result in an efficient fusion (FIG. 12E). Only fused cardioids from day 3.5 were contracting in synchrony (FIG. 5G), demonstrating that the different cardioid subtypes interact functionally. Hereafter, we refer to these structures as multi-chambered cardioids. Multi-chambered cardioids could form in all combinations, allowing us to study the interactions of two-chambered cardioids (FIG. 121) or three-chambered cardioids (atrial, LV, RV fusion) in the same order as within the developing embryonic heart (FIGS. 5F-H).

The directional electrochemical signal propagation occurs early in heart development and has not been tracked in human embryos. The directionality of electrochemical signal and fluid propagation is gradually established, initially without pacemakers, valves and septa. First electrochemical signals appear in the differentiating FHF/LV. Once the atrial region develops, it paces the other areas, ensuring the unidirectional signal motion and flow from the atria over the LV to the RV and OFT. We first measured the calcium signal propagation and tracked the signal propagation on MEAs in the multi-chambered cardioid system to investigate whether it recapitulates this process. We found that each beat originated only from one location and then propagated through the entire multi-chambered cardioid (FIGS. 5G and 12G-H), ensuring a unidirectional flow of signal propagation. On day 6.5, most beats originate from the LV (FIG. 5K), including pacing the RV, which does not beat independently (FIG. 51). We validated these observations in different multi-chambered cardioids showing that multi-chambered cardioids paced by the LV maintain the same beat frequency as LV cardioids (FIG. 5J). Interestingly, on day 6.5, the pacing potential seems limited as the LV region could not pace the three-chambered cardioids, and the atria could not pace a multi-chambered cardioid (FIGS. 5I and 5L). However, as the multi-chamber cardioids developed to day 9.5, the signal originated almost exclusively from the atrial region in all combinations (FIG. 5K). Thus, we have demonstrated that multi-chambered cardioids provide a unique tool for deciphering the ontogeny of electrochemical signal propagation throughout co-developing cardiac chambers. As heart chambers co-develop, they initially share a lumen before septation and the formation of valves. Therefore, we further optimized the co-development of a shared lumen in multi-chambered cardioids. Strikingly, when we combined developing FHF, aSHF and pSHF cardioids just before the formation of cavities (FIG. 5L) (day 1.5 for FHF/LV and day 3.5 for aSHF/RV and pSHF/atrial), they co-developed a shared lumen and still retained their identity (FIGS. 5L-0). Taken together, we developed a human compartment-specific and multi-chamber platform to comprehensively dissect the earliest aspects of human cardiogenesis.

### Example 8: Mutations Cause Compartment-Specific Defects in Cardioids

Mutations of cardiac transcription factors (TF) often cause compartment-specific congenital defects of heart development. To genetically validate the cardioid compartment platform, we generated knockout (KO) hPSC lines for important cardiac TF (ISL1, TBX5) known to lead to compartment-specific defects in vivo. Moreover, we used our system to investigate the KO effects of the TF FOXF1.

ISL1 is a prominent TF whose disruption causes severe cardiac malformations in the OFT and RV, partial defects in the atria, and lethality in mice at E10.5. To investigate the phenotype in the human cardioid compartment platform, we developed ISL1-KO hPSCs (FIG. 13A) and differentiated them into LV, RV, atrial, and OFT subtypes (FIG. 6A). Misregulation of gene ex-pression was already visible at day 3.5 in ISL1 KOs as seen by lower levels of key cardiac TFs MEF2C, NKX2-5 and MYOCD, suggesting slower differentiation progression (FIG. 13B). We saw the most drastic gene expression changes in the OFT cardioids, with HAND2 and BMP4 being down-regulated and TBX5 being up-regulated. In atrial cardioids, HOXB1, a key pSHF marker, was down-regulated (FIG. 13B). A time-course analysis from day 2.5 to 9.5 revealed that the ISL1 KO cardioids showed the most severe morphogenetic changes starting from day 4.5 in the OFT and atrial cardioids, while the impact on RV and LV cardioids is less visible (FIG. 13E). On day 9.5, there was a significant size difference in all cardioid subtypes (FIGS. 6A and 13F). The morphological defect at day 9.5 in the KO is also reflected in the misregulation of NPPA, NPPB, NR2F2, HEY1, RSPO3, WNT5A and MYL7 different cardioid subtypes (FIG. 6C). The efficiency of CM differentiation was lower in all lineages except the LV (FIG. 6B). Specifically, OFT and atrial KO cardioids showed areas without TNNT2 expression, but atrial cardioids still maintained their identity, although delayed in differentiation and contraction (FIGS. 6B-D and 13D). In contrast, major regulators of the OFT were misregulated, causing a global gene expression shift from OFT identity to atrial identity (FIG. 6D, 6F, 13C). This was also confirmed by the contraction analysis of ISL-KO OFT cardioids, which acquire atrial-like beating behavior at day 14.5 (FIG. 6E).

Another prominent cardiac TF is TBX5, a key regulator in FHF and pSHF progenitors and responsible for promoting the chamber gene expression program. Disruption of TBX5 leads to atrial and ventricular septal defects, conduction defects, and mutations that cause the Holt-Oram syndrome in humans. In the TBX5 KO (FIG. 13H), we observed that HOXB1 expression was diminished in atrial and AVC cardioids at day 3.5 (FIGS. 6G and 13G). Global gene expression analysis revealed that a range of aSHF markers (TBX1, FOXC2, FGF10) was upregulated in TBX5 KO atrial cardioids (FIG. 13G), and TBX2/3 were downregulated in AVC cardioids at day 3.5 (FIG. 131). Moreover, TBX5 KOs differentiated into the FHF lineage showed an upregulation of HAND2, GATA4 and ISL1 (FIG. 13G). In contrast, in aSHF-derived cardioids, no major defects were not detected, in line with findings in vivo (FIG. 13G). Consistent with these results, on day 9.5, we observed the most severe morphogenetic phenotypes for LV, atrial and AVC cardioids, whereas RV cardioids only showed a slight decrease in size (FIGS. 6H and 6H′). Atrial and AVC cardioids fail to differentiate into CMs, as seen by the absence Of TNNT2 (FIG. 61). LV and RV cardioids showed inefficient CM differentiation, and the chamber-specific marker NPPA was down-regulated (FIG. 61). All TBX5 KO cardioid subtypes showed a prominent defect in the ventricular chamber marker expression like NPPA/B, IRX3/4/1, HEY2 and upregulation of non-chamber marker TBX2 in RV and LV cardioids (FIG. 6J). Overall, the TBX5 KO showed specific phenotypes at different stages; LV, atrial, and AVC cardioids are already affected at the progenitor stage and fail to form CMs, whereas LV and RV cardioids showed a mild phenotype at the CM specification stage when chamber specific genes were downregulated.

The Forkhead box transcription factor (FOXF1) is a specific regulator of the pSHF lineage. Disruption of FOXF1 leads mainly to atrial septation defects, but mutant mice already die by E8.0 due to extraembryonic mesoderm defects. The broad expression of this TF in the early mesoderm is consistent with its possible role in establishing cardiovascular progenitor identity in the early lateral mesoderm. We analyzed FOXF1 KO cardioids (FIG. 13J) in all subtypes (FIG. 6N) and observed a severe morphological phenotype starting from day 3.5 in atrial and AVC cardioids (FIG. 6L). The main pSHF markers (HOXB1, OSR1) and AVC markers (TBX2, TBX3) were downregulated (FIGS. 6K and 6M), consistent with pSHF specification failure. LV cardioid KOs showed downregulation of TBX5, while RV cardioids showed an upregulation of TBX1 (FIG. 6K). On day 9.5, we observed morphological phenotypes in the LV and AVC KO cardioids (FIGS. 6N and 13K). Interestingly, atrial cardioids shifted to a more ventricular identity (IRX1+, IRX4+, HEY2+, NRF2F2-) (FIGS. 13M and 13N), while AVC cardioids failed to differentiate efficiently (FIG. 13L). As expected, we did not observe a phenotype in the RV, except for the downregulation of NPPA, seen in all subtypes (FIGS. 13L and 13M). A less severe phenotype appeared in the LV cardioids, where genes involved in cardiac contraction (ENO1, ENO2) were downregulated (FIG. 13M), confirmed by functional assays where the KO LV cardioids showed a lower beating rate (FIG. 60). KO atrial cardioids also showed a lower beating rate, while AVC cardioids did not contract (FIG. 60). In summary, the cardioid platform can be employed to dissect stage- and compartment-specific genetic cardiac defects.

### Example 9: A Multi-Chamber Cardioid Platform for Screening Teratogen-Induced Heart Defects

Besides genetic causes, congenital heart defects are often induced by teratogens (e. g., drugs, toxins, metabolites). However, screening for compartment-specific teratogenic effects in an efficient, high-throughput and easily quantifiable human system is lacking. To test the teratogenic applicability of the fused cardioids, we first confirmed the non-teratogenic factor (Aspirin, high 30 μM dosage) as a negative control. We have neither observed morphological nor significant gene expression differences (FIGS. 14A and 14B) in the cardioid system. Next, we tested thalidomide, a well-known teratogen in humans but not rodents. Thalidomide binds to TBX5, causing severe cardiac septal defects, downregulation of chamber markers and limb malformations. We used the high-throughput platform to dissect the effects of thalidomide at different concentrations (0.1 μM, 1 μM, 10 μM compared to human therapeutic plasma concentrations at ˜2.4 μM). We observed a dosage-specific impact in different compartments, with the most striking effect on AVC formation, which was not detected before (FIGS. 7A and 7A′). At the same time, RV and OFT cardioids were less affected (FIGS. 7A and 7A′). The gene expression profile of the treatments revealed a downregulation of the chamber-specific marker NPPA for all lineages except for the RV and OFT and a dosage-specific misregulation of compartment identity markers (NR2F2, IRX1/4) (FIG. 7B). These results demonstrate and validate that the multi-compartment platform can discern the early compartment-specific effect of teratogenic drugs in a human system and relate these to defects seen in patients.

Another class of compounds known to induce congenital defects is retinoid derivatives used in leukemia, psoriasis, acne creams, etc., that cause AVC defects and OFT-derivative defects. Since RA plays a crucial role in vivo and in our cardioid system, we hypothesized that the cardioid compartment platform would allow the stage-specific dissection of the underlying mechanisms. We tested acitretin and isotretinoin and observed severe compartment-specific and stage-specific effects at strikingly low dosages (e. g. 5 nM Acitretin atrial; 1 nM Isotretinoin in LV). OFT, atrial, and AVC cardioids had specification, patterning and morphogenesis defects (FIGS. 7C, 7D, 14C, 14C′). Surprisingly, when using Transretinol, we only saw a severe morphological effect on OFT cardioids, while all the other cardioid subtypes were unaffected (FIGS. 14E and 14E′). In OFT cardioids, retinoids caused downregulation of OFT genes (WNT5A, MSX1, ISL1) and upregulation of ventricular and chamber genes (IRX1, IRX4, NPPA), but not atrial genes (FIGS. 7E and 14F). Moreover, OFT cardioids treated with retinoids differentiate earlier into CMs (FIGS. 7C and 14D). Taken together, we further validated the cardioid compartment platform and used it to dissect drug-specific effects on different compartments with high sensitivity.

Different plastic residues are a neglected but ubiquitous class of compounds in our environment with possible teratogenic effects. The in vivo effects of plastic residues are very difficult to demonstrate since, until now, we did not have a validated system to unravel specification and morphogenesis defects in the human heart. Here we use our cardioid compartment platform to investigate the effect of different combinations of plastic residues (BPAs, PFOs, and nano plastics). Upon treatment with plastic residues, we observed a severe delay in RV cardioid specification and inefficient RV and atrial cardioid differentiation, while LV and AVC cardioids were unaffected (FIGS. 7F and 14G). This highlights that we have a system to comprehensively test the teratogenic effects of ubiquitously present environmental compounds on human cardiac development.

### Discussion

We developed a signaling-controlled cardioid platform representing all major compartments of the embryonic heart. In this system, the three different progenitor populations (aSHF, pSHF, and FHF) give rise to five major cardiac compartments separately or in combination (LV, RV, OFT, Atria, and AVC), mimicking selected aspects of early human heart development. The 2D to 3D differentiation approach ensures homogenous progenitor specification early, reducing heterogeneity and increasing robustness, which remains a challenge in the organoid field. As a result, the platform is highly efficient, reproducible, works with multiple cell lines, is screenable in high throughput applications, and versatile, comprising single compartment or multi-chamber cardioids.

In vivo, the dosage and timing of signaling drives specification of these lineages already during mesoderm induction in the primitive streak and as mesodermal cells migrate from it at different times and take defined positions within the heart fields. Consistently, we found that specific Activin/Nodal and WNT signaling activation levels drive specification into distinct SHF, AVC and FHF progenitors. Following mesoderm induction, inhibiting TGF-beta signaling enables the determination of the SHF lineage fate, which is consistent with the signaling environment in the anterior region of the embryo. The specific combinations of mesoderm induction and patterning signals allow mimicking the identities, dynamics, and later functionality of the cardiac lineages in development. For instance, both SHF lineages show delayed cavity formation and differentiation into CMs, and the aSHF is more epithelial and highly proliferative than the FHF. In contrast to the aSHF and in agreement with in vivo observations, the pSHF encompasses a more diverse range of induction and patterning conditions, resulting in either AVC or atrial phenotypes. The early in vivo-like functionality is also reflected by the contraction differences and dynamics of atrial, AVC, LV, RV, and OFT cardioids, as pacemakers are still absent at this developmental stage. Finally, with its different induction and patterning stages, the fused cardioids allow for the dissection of progenitor and compartment sorting mechanisms and chamber interactions.

We found that the role of RA signaling in the specification of the different lineages is more complex in terms of dosage and timing than previously thought. The absence of RA signaling is characteristic for initial aSHF specification and later OFT differentiation. Relatively low levels of RA are required for LV specification and high levels of RA for atrial specification early on. However, the timing is also important as the aSHF RV specification requires high levels of RA signaling at later stages. The teratogenic screening experiments confirmed the critical role of RA signaling dosage, showing the strong effect of retinoids on differentiation speed, specification efficiency and direction, morphogenesis, and physiology leading to compartment-specific defects.

Interactions between cardiac lineages during the earliest stages of heart development, including cardiac mesoderm specification, morphogenesis, and functional differentiation, are notoriously difficult to analyze in embryos and impossible to access in early human embryos. However, this aspect is the key to understanding the impact of mutations and teratogens on early heart development and how they lead to defects in the specification, morphogenesis and contraction signal propagation, resulting in embryo failure. For instance, it is unclear how the different compartments sort and remain separate, which can now be addressed using the multi-chamber cardioid platform. Another crucial and neglected aspect of cardiac development is the ontology of contraction signal propagation through the different early stages (day 20-30) of human heart development. This is particularly important to understand the cardiac causes of embryo failure that have been attributed to faulty specification and morphogenesis but could also be caused by early contraction signal propagation defects between chambers.

The fused cardioids allow us to comprehensively and systematically dissect unidentified mutations in regulatory elements such as enhancers, environmental factors such as pollutants, diet and complex interactions between genetic and environmental factors.

1-15. (canceled)

16. A cardiac organoid comprising: a heart tissue with at least one inner cavity or a central chamber, wherein the cardiac organoid comprises at least two different heart tissues selected from left ventricle tissue, right ventricle tissue, atrial tissue, outflow tract tissue, atrioventricular canal tissue, sinoatrial node tissue, and atrioventricular node tissue, wherein the central chamber can be shared by at least two different heart tissues, and wherein the at least two different heart tissues comprise a electrophysiological or calcium signaling connection and/or ability to propagate a tissue contraction;
and wherein either i) the cardiac organoid has the central chamber and said central chamber is shared by at least two different heart tissues, or ii) the cardiac organoid has at least two different heart tissues with the ability to propagate a contraction with a beating behaviour that starts in one of the different heart tissues and propagates or projects into a neighbouring heart tissue, or both i) and ii),
wherein the cardiac organoid is obtainable from differentiating mesoderm cells.

17. The cardiac organoid of claim 16, wherein
the left ventricle tissue comprises at least 60% cardiac cells selected from cardiomyocytes, endocardial cells and epicardial cells;
the right ventricle tissue comprises at least 60% cardiomyocytes;
the atrial tissue comprises at least 60% cardiomyocytes;
the outflow tract tissue comprises at least 60% cardiomyocytes;
the atrioventricular canal tissue comprises at least 60% cardiomyocytes;
the sinoatrial node tissue comprises at least 60% cardiomyocytes; and/or
atrioventricular node tissue comprises at least 60% cardiomyocytes.

18. The cardiac organoid of claim 16, wherein the inner cavity or central chamber is completely surrounded by tissue selected from left ventricle tissue, right ventricle tissue, atrial tissue, outflow tract tissue, atrioventricular canal tissue, sinoatrial node tissue, or atrioventricular node tissue; and/or wherein the volume of the inner cavity or central chamber is not leading into a major blood vessel.

19. The cardiac organoid of claim 16 having a size in its largest dimension of 0.3 mm to 50 mm.

20. The cardiac organoid of claim 16, wherein
left ventricle tissue cells express one or more expression markers selected from NPPA, IRX4 and HEY2; and/or left ventricle tissue cells lack expression of one or more expression markers selected from NR2F2, TBX2 and TBX3;
right ventricle tissue cells express one or more expression markers selected from NPPA, IRX1, IRX2 and PRDX1; and/or right ventricle tissue cells lack expression of one or more expression markers selected from NR2F2, TBX2, and WNT5A;
atrial tissue cells express one or more expression markers selected from NPPA, NR2F1, NR2F2 and HEY1; and/or atrial tissue cells lack expression of one or more expression markers selected from IRX1, IRX4 and HEY2;
outflow tract tissue cells express one or more expression markers selected from WNT5A, MSX1, BMP4, WNT11 and RSPO3; and/or outflow tract tissue cells lack expression of one or more expression markers selected from TBX3, NR2F1 and NPPA;
atrioventricular canal tissue cells express one or more expression markers selected from TBX2, MSX2 and RSPO3; and/or atrioventricular canal tissue cells lack expression of one or more expression markers selected from IRX1, IRX4 and NPPA;
sinoatrial node tissue cells express one or more expression markers selected from SHOX2, TBX3, HCN4, ISL1 and GJC1; and/or sinoatrial node tissue cells lack expression of one or more expression markers selected from NKX2.5, IRX1, IRX4 and NPPA; and/or
atrioventricular node tissue cells express one or more expression markers selected from TBX3, TBX5, KCNE1, HCN4 and GJC1; and/or atrioventricular node tissue cells lack expression of one or more expression markers selected from RSPO3, MSX2, IRX4 and NPPA.

21. The cardiac organoid of claim 16, wherein the size of the inner cavity or central chamber at its largest dimension is at least 30% of the size of the cardiac organoid at its largest dimension.

22. A method to generate a cardiac organoid of claim 16 comprising generating at least two different heart tissues in vitro, wherein the different heart tissues are selected from left ventricle progenitor first heart field tissue, right ventricle/outflow tract progenitor anterior second heart field tissue, right ventricle progenitor anterior second heart field tissue, atrial progenitor posterior second heart field tissue, outflow tract progenitor anterior second heart field tissue, atrioventricular canal progenitor posterior second heart field tissue, sinoatrial node progenitor posterior second heart field tissue, and atrioventricular node tissue, and fusing the at least two heart tissues,
culturing the fused tissue model and letting calcium signaling connection, ability to propagate a tissue contraction and/or a central chamber between the different heart tissues form.

23. The method of claim 22, wherein the different heart tissues have been cultured and differentiated from a pluripotent cell and wherein the fusion is at culture day 1 to 7 from a pluripotent stage; right ventricle/outflow tract progenitor anterior second heart field tissue, right ventricle progenitor second heart field tissue, atrial progenitor second heart field tissue, atrioventricular progenitor second heart field canal tissue, sinoatrial node progenitor second heart field tissue, and/or atrioventricular node tissue is fused at culture day 2 to 5; or left ventricle progenitor first heart field tissue is fused when expressing the expression marker TBX5 and/or HAND1; right ventricle/outflow tract progenitor anterior second heart field tissue or right ventricle progenitor anterior second heart field tissue is fused when expressing the expression marker TBX1, FOXC1 and/or FOXC2; atrial progenitor posterior second heart field tissue is fused when expressing the expression marker HOXB1, TBX5 and/or OSR1; atrioventricular canal progenitor posterior second heart field tissue is fused when expressing the expression marker TBX3, FOXF1 and/or HOXB1;
sinoatrial node progenitor posterior second heart field tissue is fused when expressing the expression marker SHOX2, TBX3, HCN4, ISL1 and/or GJC1; and/or
atrioventricular node tissue is fused when expressing the expression marker TBX3, TBX5, KCNE1, HCN4 and/or GJC1.

24. The method of claim 22,
wherein one of the at least two different heart tissues is left ventricle progenitor first heart field tissue and generating left ventricle progenitor first heart field tissue comprises differentiating mesoderm cells into left ventricular precursor cells in a medium comprising a bone morphogenic protein BMP4, a fibroblast growth factor FGF2, insulin, a Wnt inhibitor Wnt-C59 or IWP2, and retinoic acid having a concentration of 5 nM to 100 nM, in the medium;
wherein one of the at least two different heart tissues is right ventricle/outflow tract progenitor anterior second heart field tissue and generating right ventricle/outflow tract progenitor anterior second heart field tissue comprises differentiating mesoderm cells into right ventricular and/or outflow tract precursor cells in a medium comprising a TGF-beta inhibitor SB 431542, and a Wnt inhibitor Wnt-C59 or XAV-939;
wherein one of the at least two different heart tissues is outflow tract progenitor anterior second heart field tissue and generating outflow tract progenitor second heart field tissue comprises differentiating mesoderm cells into outflow tract tissue precursor cells in a medium comprising a TGF-beta inhibitor SB 431542, and a Wnt inhibitor Wnt-C59 or XAV-939;
wherein one of the at least two different heart tissues is atrial progenitor posterior second heart field tissue and generating atrial progenitor posterior second heart field tissue comprises differentiating mesoderm cells into atrial tissue precursor cells in a medium comprising a TGF-beta inhibitor SB 431542, a Wnt inhibitor Wnt-C59 or XAV-939, and retinoic acid in a concentration of 300 nM to 800 nM; and/or
wherein one of the at least two different heart tissues is atrioventricular canal progenitor second heart field tissue and generating atrioventricular canal progenitor posterior second heart field tissue comprises differentiating mesoderm cells into atrioventricular canal tissue precursor cells in a medium comprising a TGF-beta inhibitor SB 431542, a Wnt inhibitor Wnt-C59 or XAV-939, a bone morphogenic protein BMP4 and retinoic acid in a concentration of 300 nM to 800 nM;
wherein one of the at least two different heart tissues is sinoatrial node progenitor posterior second heart field tissue and generating sinoatrial node progenitor posterior second heart field tissue comprises differentiating mesoderm cells into sinoatrial node tissue precursor cells in a medium comprising a TGF-beta inhibitor SB 431542, a bone morphogenic protein, BMP4 and retinoic acid in a concentration of 300 nM to 800 nM; and/or
wherein one of the at least two different heart tissues is atrioventricular node tissue and generating atrioventricular node tissue comprises differentiating mesoderm cells into atrioventricular canal tissue precursor cells in a medium comprising a TGF-beta inhibitor SB 431542, a Wnt inhibitor Wnt-C59 or XAV-939, a bone morphogenic protein BMP4 and retinoic acid in a concentration of 300 nM to 800 nM; and wherein atrioventricular canal progenitor posterior second heart field tissue is further differentiated into atrioventricular node tissue by further maturing in a medium containing an activator of sonic hedgehog signaling and/or a BMP.

25. The method of claim 22, wherein fusing the at least two heart tissues comprises culturing in a medium comprising a Wnt inhibitor, a bone morphogenic protein, a fibroblast growth factor, insulin, and retinoic acid, the retinoic acid is in a concentration of 300 nM to 800 nM.

26. The method of claim 22 for screening or testing a candidate compound on its effects on heart development and/or functionality comprising generating a cardiac organoid while treating the cells with the candidate compound and comparing development of the cardiac organoid with development and/or or functionality of a cardiac organoid that was not treated with the candidate compound.

27. A method of observing the effects of suppressed, mutated or overexpressed genes during on heart development comprising generating a cardiac organoid according to claim 22 wherein the cells have a suppressed or mutated candidate gene or overexpress a candidate gene and comparing development of the cardiac organoid with development of a cardiac organoid that was not generated with a suppressed, mutated or overexpressed gene.

28. A method of screening or testing a candidate compound on its effects on heart functionality comprising treating a cardiac organoid according to claim 16 with the candidate compound and comparing with a functionality of a cardiac organoid that was not treated with the candidate compound.

29. A method of treating a heart injury in a patient comprising transplanting a cell, from a cardiac organoid of claim 16 to the injury.

30. Use of a cell culture medium comprising
a) a bone morphogenic protein BMP4, a fibroblast growth factor FGF2, insulin, a Wnt inhibitor Wnt-C59 or XAV-939, and retinoic acid having a concentration of less than 100 nM;
b) a TGF-beta inhibitor SB 431542, and a Wnt inhibitor Wnt-C59 or XAV-939;
c) a Wnt inhibitor Wnt-C59 or XAV-939, a bone morphogenic protein BMP4, a fibroblast growth factor FGF2, insulin, and retinoic acid having a concentration of 50 nM to 500 nM in the medium;
d) a TGF-beta inhibitor SB 431542, and a Wnt inhibitor Wnt-C59 or XAV-939;
e) a Wnt inhibitor Wnt-C59 or XAV-939, a bone morphogenic protein, BMP4, a fibroblast growth factor FGF2, insulin, and the medium lacking retinoic acid;
f) a TGF-beta inhibitor SB 431542, a Wnt inhibitor Wnt-C59 or XAV-939, and retinoic acid in a concentration of 300 nM to 800 nM;
g) a Wnt inhibitor Wnt-C59 or XAV-939, a bone morphogenic protein BMP4, a fibroblast growth factor FGF2, insulin, and retinoic acid in a concentration of 300 nM to 800 nM;
h) a TGF-beta inhibitor SB 431542, a Wnt inhibitor Wnt-C59 or XAV-939, a bone morphogenic protein BMP4 and retinoic acid in a concentration of 300 nM to 800 nM;
i) activin and CHIR99021; the activin is at a concentration of 1 ng/ml to 8 ng/ml, and/or the CHIR99021 is at a concentration of 1 μM to 6 μM; or
j) a Wnt inhibitor Wnt-C59, a bone morphogenic protein BMP4, a fibroblast growth factor FGF2, insulin, and retinoic acid in a concentration of 300 nM to 800 nM;
in the method according to claim 22.
